SEQUENCE LISTING (139 sequences)

SEQ ID NO: 1 - DNA, 6 bp, Artificial Sequence
  Description of Artificial Sequence Synthetic oligonucleotide
aaaact

SEQ ID NO: 2 - DNA, 6 bp, Artificial Sequence
  Description of Artificial Sequence Synthetic oligonucleotide
agatta

SEQ ID NO: 3 - DNA, 6 bp, Artificial Sequence
  Description of Artificial Sequence Synthetic oligonucleotide
ataaac

SEQ ID NO: 4 - DNA, 6 bp, Artificial Sequence
  Description of Artificial Sequence Synthetic oligonucleotide
atacaa

SEQ ID NO: 5 - DNA, 6 bp, Artificial Sequence
  Description of Artificial Sequence Synthetic oligonucleotide
aaagtt

SEQ ID NO: 6 - DNA, 6 bp, Artificial Sequence
  Description of Artificial Sequence Synthetic oligonucleotide
aaattg

SEQ ID NO: 7 - DNA, 6 bp, Artificial Sequence
  Description of Artificial Sequence Synthetic oligonucleotide
aagatt

SEQ ID NO: 8 - DNA, 6 bp, Artificial Sequence
  Description of Artificial Sequence Synthetic oligonucleotide
aataca

SEQ ID NO: 9 - DNA, 6 bp, Artificial Sequence
  Description of Artificial Sequence Synthetic oligonucleotide
aatctt

SEQ ID NO: 10 - DNA, 6 bp, Artificial Sequence
  Description of Artificial Sequence Synthetic oligonucleotide
aattct

SEQ ID NO: 11 - DNA, 6 bp, Artificial Sequence
  Description of Artificial Sequence Synthetic oligonucleotide
acaata

SEQ ID NO: 12 - DNA, 6 bp, Artificial Sequence
  Description of Artificial Sequence Synthetic oligonucleotide
acttat

SEQ ID NO: 13 - DNA, 6 bp, Artificial Sequence
  Description of Artificial Sequence Synthetic oligonucleotide
atatag

SEQ ID NO: 14 - DNA, 6 bp, Artificial Sequence
  Description of Artificial Sequence Synthetic oligonucleotide
ctttaa

SEQ ID NO: 15 - DNA, 6 bp, Artificial Sequence
  Description of Artificial Sequence Synthetic oligonucleotide
gataat

SEQ ID NO: 16 - DNA, 6 bp, Artificial Sequence
  Description of Artificial Sequence Synthetic oligonucleotide
gtaata

SEQ ID NO: 17 - DNA, 6 bp, Artificial Sequence
  Description of Artificial Sequence Synthetic oligonucleotide
atcaaa

SEQ ID NO: 18 - DNA, 6 bp, Artificial Sequence
  Description of Artificial Sequence Synthetic oligonucleotide
atgaat

SEQ ID NO: 19 - DNA, 6 bp, Artificial Sequence
  Description of Artificial Sequence Synthetic oligonucleotide
attact

SEQ ID NO: 20 - DNA, 6 bp, Artificial Sequence
  Description of Artificial Sequence Synthetic oligonucleotide
attcta

SEQ ID NO: 21 - DNA, 6 bp, Artificial Sequence
  Description of Artificial Sequence Synthetic oligonucleotide
atttca

SEQ ID NO: 22 - DNA, 6 bp, Artificial Sequence
  Description of Artificial Sequence Synthetic oligonucleotide
caaata

SEQ ID NO: 23 - DNA, 6 bp, Artificial Sequence
  Description of Artificial Sequence Synthetic oligonucleotide
cattat

SEQ ID NO: 24 - DNA, 6 bp, Artificial Sequence
  Description of Artificial Sequence Synthetic oligonucleotide
ctatat

SEQ ID NO: 25 - DNA, 6 bp, Artificial Sequence
  Description of Artificial Sequence Synthetic oligonucleotide
gtttat

SEQ ID NO: 26 - DNA, 6 bp, Artificial Sequence
  Description of Artificial Sequence Synthetic oligonucleotide
tcatat

SEQ ID NO: 27 - DNA, 6 bp, Artificial Sequence
  Description of Artificial Sequence Synthetic oligonucleotide
tcttaa

SEQ ID NO: 28 - DNA, 6 bp, Artificial Sequence
  Description of Artificial Sequence Synthetic oligonucleotide
tgattt

SEQ ID NO: 29 - DNA, 6 bp, Artificial Sequence
  Description of Artificial Sequence Synthetic oligonucleotide
taaagt

SEQ ID NO: 30 - DNA, 6 bp, Artificial Sequence
  Description of Artificial Sequence Synthetic oligonucleotide
taagat

SEQ ID NO: 31 - DNA, 6 bp, Artificial Sequence
  Description of Artificial Sequence Synthetic oligonucleotide
taatga

SEQ ID NO: 32 - DNA, 6 bp, Artificial Sequence
  Description of Artificial Sequence Synthetic oligonucleotide
tactat

SEQ ID NO: 33 - DNA, 6 bp, Artificial Sequence
  Description of Artificial Sequence Synthetic oligonucleotide
tagtaa

SEQ ID NO: 34 - DNA, 6 bp, Artificial Sequence
  Description of Artificial Sequence Synthetic oligonucleotide
tataga

SEQ ID NO: 35 - DNA, 6 bp, Artificial Sequence
  Description of Artificial Sequence Synthetic oligonucleotide
tatgaa

SEQ ID NO: 36 - DNA, 6 bp, Artificial Sequence
  Description of Artificial Sequence Synthetic oligonucleotide
tattgt

SEQ ID NO: 37 - DNA, 6 bp, Artificial Sequence
  Description of Artificial Sequence Synthetic oligonucleotide
tgttta

SEQ ID NO: 38 - DNA, 6 bp, Artificial Sequence
  Description of Artificial Sequence Synthetic oligonucleotide
caccaa

SEQ ID NO: 39 - DNA, 6 bp, Artificial Sequence
  Description of Artificial Sequence Synthetic oligonucleotide
ccacat

SEQ ID NO: 40 - DNA, 6 bp, Artificial Sequence
  Description of Artificial Sequence Synthetic oligonucleotide
ctagtg

SEQ ID NO: 41 - DNA, 6 bp, Artificial Sequence
  Description of Artificial Sequence Synthetic oligonucleotide
ttaatc

SEQ ID NO: 42 - DNA, 6 bp, Artificial Sequence
  Description of Artificial Sequence Synthetic oligonucleotide
ttagtt

SEQ ID NO: 43 - DNA, 6 bp, Artificial Sequence
  Description of Artificial Sequence Synthetic oligonucleotide
ttattg

SEQ ID NO: 44 - DNA, 6 bp, Artificial Sequence
  Description of Artificial Sequence Synthetic oligonucleotide
ttgaaa

SEQ ID NO: 45 - DNA, 6 bp, Artificial Sequence
  Description of Artificial Sequence Synthetic oligonucleotide
tttaca

SEQ ID NO: 46 - DNA, 6 bp, Artificial Sequence
  Description of Artificial Sequence Synthetic oligonucleotide
tttctt

SEQ ID NO: 47 - DNA, 6 bp, Artificial Sequence
  Description of Artificial Sequence Synthetic oligonucleotide
ttttga

SEQ ID NO: 48 - DNA, 6 bp, Artificial Sequence
  Description of Artificial Sequence Synthetic oligonucleotide
agacct

SEQ ID NO: 49 - DNA, 6 bp, Artificial Sequence
  Description of Artificial Sequence Synthetic oligonucleotide
gctaga

SEQ ID NO: 50 - DNA, 6 bp, Artificial Sequence
  Description of Artificial Sequence Synthetic oligonucleotide
aggggc

SEQ ID NO: 51 - DNA, 6 bp, Artificial Sequence
  Description of Artificial Sequence Synthetic oligonucleotide
cacggc

SEQ ID NO: 52 - DNA, 6 bp, Artificial Sequence
  Description of Artificial Sequence Synthetic oligonucleotide
cagggg

SEQ ID NO: 53 - DNA, 6 bp, Artificial Sequence
  Description of Artificial Sequence Synthetic oligonucleotide
gggatt

SEQ ID NO: 54 - DNA, 6 bp, Artificial Sequence
  Description of Artificial Sequence Synthetic oligonucleotide
gttcga

SEQ ID NO: 55 - DNA, 6 bp, Artificial Sequence
  Description of Artificial Sequence Synthetic oligonucleotide
tctgca

SEQ ID NO: 56 - DNA, 6 bp, Artificial Sequence
  Description of Artificial Sequence Synthetic oligonucleotide
ccaacc

SEQ ID NO: 57 - DNA, 6 bp, Artificial Sequence
  Description of Artificial Sequence Synthetic oligonucleotide
gtaccg

SEQ ID NO: 58 - DNA, 6 bp, Artificial Sequence
  Description of Artificial Sequence Synthetic oligonucleotide
accggc

SEQ ID NO: 59 - DNA, 6 bp, Artificial Sequence
  Description of Artificial Sequence Synthetic oligonucleotide
acgggg

SEQ ID NO: 60 - DNA, 6 bp, Artificial Sequence
  Description of Artificial Sequence Synthetic oligonucleotide
agcggg

SEQ ID NO: 61 - DNA, 6 bp, Artificial Sequence
  Description of Artificial Sequence Synthetic oligonucleotide
cccacg

SEQ ID NO: 62 - DNA, 6 bp, Artificial Sequence
  Description of Artificial Sequence Synthetic oligonucleotide
cgctgc

SEQ ID NO: 63 - DNA, 6 bp, Artificial Sequence
  Description of Artificial Sequence Synthetic oligonucleotide
cggcca

SEQ ID NO: 64 - DNA, 6 bp, Artificial Sequence
  Description of Artificial Sequence Synthetic oligonucleotide
cgggct

SEQ ID NO: 65 - DNA, 6 bp, Artificial Sequence
  Description of Artificial Sequence Synthetic oligonucleotide
ccccgt

SEQ ID NO: 66 - DNA, 6 bp, Artificial Sequence
  Description of Artificial Sequence Synthetic oligonucleotide
ccctgg

SEQ ID NO: 67 - DNA, 6 bp, Artificial Sequence
  Description of Artificial Sequence Synthetic oligonucleotide
ccggac

SEQ ID NO: 68 - DNA, 6 bp, Artificial Sequence
  Description of Artificial Sequence Synthetic oligonucleotide
ccgtcg

SEQ ID NO: 69 - DNA, 6 bp, Artificial Sequence
  Description of Artificial Sequence Synthetic oligonucleotide
cctggc

SEQ ID NO: 70 - DNA, 6 bp, Artificial Sequence
  Description of Artificial Sequence Synthetic oligonucleotide
cgaggc

SEQ ID NO: 71 - DNA, 6 bp, Artificial Sequence
  Description of Artificial Sequence Synthetic oligonucleotide
cgccct

SEQ ID NO: 72 - DNA, 6 bp, Artificial Sequence
  Description of Artificial Sequence Synthetic oligonucleotide
cgcgca

SEQ ID NO: 73 - DNA, 6 bp, Artificial Sequence
  Description of Artificial Sequence Synthetic oligonucleotide
cggtgg

SEQ ID NO: 74 - DNA, 6 bp, Artificial Sequence
  Description of Artificial Sequence Synthetic oligonucleotide
gcgagc

SEQ ID NO: 75 - DNA, 6 bp, Artificial Sequence
  Description of Artificial Sequence Synthetic oligonucleotide
gcgctg

SEQ ID NO: 76 - DNA, 6 bp, Artificial Sequence
  Description of Artificial Sequence Synthetic oligonucleotide
gcggtc

SEQ ID NO: 77 - DNA, 6 bp, Artificial Sequence
  Description of Artificial Sequence Synthetic oligonucleotide
cgtggg

SEQ ID NO: 78 - DNA, 6 bp, Artificial Sequence
  Description of Artificial Sequence Synthetic oligonucleotide
ctgcgg

SEQ ID NO: 79 - DNA, 6 bp, Artificial Sequence
  Description of Artificial Sequence Synthetic oligonucleotide
gaccgc

SEQ ID NO: 80 - DNA, 6 bp, Artificial Sequence
  Description of Artificial Sequence Synthetic oligonucleotide
gagcgg

SEQ ID NO: 81 - DNA, 6 bp, Artificial Sequence
  Description of Artificial Sequence Synthetic oligonucleotide
gcacgg

SEQ ID NO: 82 - DNA, 6 bp, Artificial Sequence
  Description of Artificial Sequence Synthetic oligonucleotide
gccagg

SEQ ID NO: 83 - DNA, 6 bp, Artificial Sequence
  Description of Artificial Sequence Synthetic oligonucleotide
gccctc

SEQ ID NO: 84 - DNA, 6 bp, Artificial Sequence
  Description of Artificial Sequence Synthetic oligonucleotide
gccgtg

SEQ ID NO: 85 - DNA, 6 bp, Artificial Sequence
  Description of Artificial Sequence Synthetic oligonucleotide
gctcgc

SEQ ID NO: 86 - DNA, 6 bp, Artificial Sequence
  Description of Artificial Sequence Synthetic oligonucleotide
tcccgc

SEQ ID NO: 87 - DNA, 6 bp, Artificial Sequence
  Description of Artificial Sequence Synthetic oligonucleotide
tcgggc

SEQ ID NO: 88 - DNA, 6 bp, Artificial Sequence
  Description of Artificial Sequence Synthetic oligonucleotide
tggccg

SEQ ID NO: 89 - DNA, 6 bp, Artificial Sequence
  Description of Artificial Sequence Synthetic oligonucleotide
ggagcc

SEQ ID NO: 90 - DNA, 6 bp, Artificial Sequence
  Description of Artificial Sequence Synthetic oligonucleotide
ggccac

SEQ ID NO: 91 - DNA, 6 bp, Artificial Sequence
  Description of Artificial Sequence Synthetic oligonucleotide
ggcgtc

SEQ ID NO: 92 - DNA, 6 bp, Artificial Sequence
  Description of Artificial Sequence Synthetic oligonucleotide
gggcag

SEQ ID NO: 93 - DNA, 6 bp, Artificial Sequence
  Description of Artificial Sequence Synthetic oligonucleotide
ggggac

SEQ ID NO: 94 - DNA, 6 bp, Artificial Sequence
  Description of Artificial Sequence Synthetic oligonucleotide
gggtcg

SEQ ID NO: 95 - DNA, 6 bp, Artificial Sequence
  Description of Artificial Sequence Synthetic oligonucleotide
ggtggc

SEQ ID NO: 96 - DNA, 6 bp, Artificial Sequence
  Description of Artificial Sequence Synthetic oligonucleotide
gtgcgc

SEQ ID NO: 97 - DNA, 57 bp, Artificial Sequence
  Description of Artificial Sequence Synthetic oligonucleotide
  modified_base (57)..(57): Inosine
aagcagtggtatcaacgcagagtacttttttttttttttttttttttttttttttvn

SEQ ID NO: 98 - DNA, 41 bp, Artificial Sequence
  Description of Artificial Sequence Synthetic oligonucleotide
  Description of Combined DNA/RNA Molecule Synthetic oligonucleotide
gtggtatcaacgcagagtacacgacgctcttccgatctggg

SEQ ID NO: 99 - DNA, 23 bp, Artificial Sequence
  Description of Artificial Sequence Synthetic primer
aagcagtggtatcaacgcagagt

SEQ ID NO: 100 - DNA, 53 bp, Artificial Sequence
  Description of Artificial Sequence Synthetic primer
actggagttcagacgtgtgctcttccgatctggaagacsgatgggcccttggt

SEQ ID NO: 101 - DNA, 56 bp, Artificial Sequence
  Description of Artificial Sequence Synthetic primer
actggagttcagacgtgtgctcttccgatcttgaagacagatggtgcagccacagt

SEQ ID NO: 102 - DNA, 56 bp, Artificial Sequence
  Description of Artificial Sequence Synthetic primer
actggagttcagacgtgtgctcttccgatctacagagtgacmgtggggttggcctt

SEQ ID NO: 103 - DNA, 56 bp, Artificial Sequence
  Description of Artificial Sequence Synthetic primer
actggagttcagacgtgtgctcttccgatctacagagtgaccgakggggcagcctt

SEQ ID NO: 104 - DNA, 6 bp, Artificial Sequence
  Description of Artificial Sequence Synthetic oligonucleotide
  modified_base (1)..(6): a, c, t, g, unknown or other
nnnnnn

SEQ ID NO: 105 - DNA, 51 bp, Artificial Sequence
  Description of Artificial Sequence Synthetic oligonucleotide
  modified_base (25)..(30): a, c, t, g, unknown or other
caagcagaagacggcatacgagatnnnnnngtgactggagttcagacgtgt

SEQ ID NO: 106 - DNA, 76 bp, Artificial Sequence
  Description of Artificial Sequence Synthetic oligonucleotide
ggccattatggccgggggtagctcaggaagcagagcctggagcatctccactatggcctgggctccactacttctc

SEQ ID NO: 107 - DNA, 76 bp, Artificial Sequence
  Description of Artificial Sequence Synthetic oligonucleotide
ggcctgggctccactacttctcaccctcctcgctcactgcacaggttcttgggccaattttatgctgactcagccg

SEQ ID NO: 108 - DNA, 76 bp, Artificial Sequence
  Description of Artificial Sequence Synthetic oligonucleotide
ggccaattttatgctgactcagccgcactctgtgtcggcgtctccggggaagacggtaaccatctcctgctcccgc

SEQ ID NO: 109 - DNA, 76 bp, Artificial Sequence
  Description of Artificial Sequence Synthetic oligonucleotide
cggtaaccatctcctgctcccgcagcagtggaaacattgccaccaactatgtgcagtggtaccagcagcgcccggg

SEQ ID NO: 110 - DNA, 76 bp, Artificial Sequence
  Description of Artificial Sequence Synthetic oligonucleotide
gtggtaccagcagcgcccgggcagtccccccaccactatgatctatgaaaatagtcaaaggccttctggagtccct

SEQ ID NO: 111 - DNA, 76 bp, Artificial Sequence
  Description of Artificial Sequence Synthetic oligonucleotide
gtcaaaggccttctggagtccctgatcgggtctctggctccatcgacagctcctccaattctgcctccctcaccat

SEQ ID NO: 112 - DNA, 74 bp, Artificial Sequence
  Description of Artificial Sequence Synthetic oligonucleotide
ctccaattctgcctccctcaccatctctggactgagtcctgaggacgaggctgactactactgtcagtcctatg

SEQ ID NO: 113 - DNA, 76 bp, Artificial Sequence
  Description of Artificial Sequence Synthetic oligonucleotide
gctgactactactgtcagtcctatgagggcagtactgtggttttcggcggagggaccaagctgaccgtcctaagtc

SEQ ID NO: 114 - DNA, 75 bp, Artificial Sequence
  Description of Artificial Sequence Synthetic oligonucleotide
gtactgtggttttcggcggagggaccaagctgaccgtcctaagtcagcccaaggctgccccctcggtcactctgt

SEQ ID NO: 115 - DNA, 446 bp, Artificial Sequence
  Description of Artificial Sequence Synthetic polynucleotide
tagctcaggaagcagagcctggagcatctccactatggcctgggctccactacttctcaccctcctcgctcactgcacaggttcttgggccaattttatgctgactcagccgcactctgtgtcggcgtctccggggaagacggtaaccatctcctgctcccgcagcagtggaaacattgccaccaactatgtgcagtggtaccagcagcgcccgggcagtccccccaccactatgatctatgaaaatagtcaaaggccttctggagtccctgatcgggtctctggctccatcgacagctcctccaattctgcctccctcaccatctctggactgagtcctgaggacgaggctgactactactgtcagtcctatgagggcagtactgtggttttcggcggagggaccaagctgaccgtcctaagtcagcccaaggctgcccaacggt

SEQ ID NO: 116 - DNA, 44 bp, Artificial Sequence
  Description of Artificial Sequence Synthetic primer
aaataatacgactcactataggtacacgacgctcttccgatctg

SEQ ID NO: 117 - DNA, 67 bp, Artificial Sequence
  Description of Artificial Sequence Synthetic primer
  modified_base (35)..(40): a, c, t, g, unknown or other
gtctcgtgggctcggagatgtgtataagagacagnnnnnngctggaccacagccgcagcgtcatgag

SEQ ID NO: 118 - DNA, 62 bp, Artificial Sequence
  Description of Artificial Sequence Synthetic primer
  modified_base (35)..(40): a, c, t, g, unknown or other
gtctcgtgggctcggagatgtgtataagagacagnnnnnnaatcctttctcttgaccatggc

SEQ ID NO: 119 - DNA, 44 bp, Artificial Sequence; identical to SEQ ID NO: 116
  Description of Artificial Sequence Synthetic primer

SEQ ID NO: 120 - DNA, 44 bp, Artificial Sequence; identical to SEQ ID NO: 116
  Description of Artificial Sequence Synthetic primer

SEQ ID NO: 121 - RNA, 24 nt, Artificial Sequence
  Description of Artificial Sequence Synthetic oligonucleotide
gguacacgacgcucuuccgaucug

SEQ ID NO: 122 - RNA, 24 nt, Artificial Sequence
  Description of Artificial Sequence Synthetic oligonucleotide
gguacacgacgcucuuccgaucug

SEQ ID NO: 123 - DNA, 36 bp, Artificial Sequence
  Description of Artificial Sequence Synthetic primer
  Description of Combined DNA/RNA Molecule Synthetic primer
agcagccgtcgcagtctacacatattctctgtcggg

SEQ ID NO: 124 - DNA, 34 bp, Artificial Sequence
  Description of Artificial Sequence Synthetic primer
gtctcgtgggctcggagatgtgtataagagacag

SEQ ID NO: 125 - DNA, 33 bp, Artificial Sequence
  Description of Artificial Sequence Synthetic primer
tcgtcggcagcgtcagatgtgtataagagacag

SEQ ID NO: 126 - DNA, 55 bp, Artificial Sequence
  Description of Artificial Sequence Synthetic primer
tcgtcggcagcgtcagatgtgtataagagacagtacacgacgctcttccgatctg

SEQ ID NO: 127 - DNA, 67 bp, Artificial Sequence; identical to SEQ ID NO: 117
  Description of Artificial Sequence Synthetic primer
  modified_base (35)..(40): a, c, t, g, unknown or other

SEQ ID NO: 128 - DNA, 64 bp, Artificial Sequence
  Description of Artificial Sequence Synthetic primer
  modified_base (35)..(40): a, c, t, g, unknown or other
gtctcgtgggctcggagatgtgtataagagacagnnnnnngaaatcctttctcttgaccatggc

SEQ ID NO: 129 - DNA, 33 bp, Artificial Sequence
  Description of Artificial Sequence Synthetic primer
ctgtctcttatacacatctgacgctgccgacga

SEQ ID NO: 130 - DNA, 55 bp, Artificial Sequence; identical to SEQ ID NO: 126
  Description of Artificial Sequence Synthetic oligonucleotide

SEQ ID NO: 131 - DNA, 55 bp, Artificial Sequence
  Description of Artificial Sequence Synthetic oligonucleotide
cagatcggaagagcgtcgtgtactgtctcttatacacatctgacgctgccgacga

SEQ ID NO: 132 - DNA, 55 bp, Artificial Sequence; identical to SEQ ID NO: 126
  Description of Artificial Sequence Synthetic oligonucleotide

SEQ ID NO: 133 - DNA, 55 bp, Artificial Sequence; identical to SEQ ID NO: 131
  Description of Artificial Sequence Synthetic oligonucleotide

SEQ ID NO: 134 - DNA, 55 bp, Artificial Sequence; identical to SEQ ID NO: 131
  Description of Artificial Sequence Synthetic oligonucleotide

SEQ ID NO: 135 - DNA, 40 bp, Artificial Sequence
  Description of Artificial Sequence Synthetic oligonucleotide
gtctcgtgggctcggagatgtgtataagagacagagtttt

SEQ ID NO: 136 - DNA, 40 bp, Artificial Sequence
  Description of Artificial Sequence Synthetic oligonucleotide
  modified_base (1)..(6): a, c, t, g, unknown or other
nnnnnnctgtctcttatacacatctccgagcccacgagac

SEQ ID NO: 137 - RNA, 60 nt, Artificial Sequence
  Description of Artificial Sequence Synthetic oligonucleotide
  modified_base (21)..(26): a, c, u, g, unknown or other
guguucccacccgaggucgcnnnnnncugucucuuauacacaucuccgagcccacgagac

SEQ ID NO: 138 - RNA, 40 nt, Artificial Sequence
  Description of Artificial Sequence Synthetic oligonucleotide
  modified_base (1)..(6): a, c, u, g, unknown or other
nnnnnncugucucuuauacacaucuccgagcccacgagac

SEQ ID NO: 139 - DNA, 40 bp, Artificial Sequence
  Description of Artificial Sequence Synthetic oligonucleotide
aaaactctgtctcttatacacatctccgagcccacgagac

(19) United States
(12) Patent Application Publication
(10) Pub. No.: US 2021/0115436 A1
(43) Pub. Date: Apr. 22, 2021

(54) METHODS FOR PREPARATION OF NUCLEIC ACID SEQUENCING LIBRARIES

(71) Applicant: BERKELEY LIGHTS, INC., Emeryville, CA (US)

(72) Inventors: Ravi K. RAMENANI, Fremont, CA (US); Duane SMITH, Berkeley, CA (US); Jason M. McEWEN, El Cerrito, CA (US); Magali SOUMILLON, Boston, MA (US)

(21) Appl. No.: 17/033,470

(22) Filed: Sep. 25, 2020

Related U.S. Application Data
(63) Continuation of application No. PCT/US2019/024623, filed on Mar. 28, 2019.
(60) Provisional application No. 62/649,482, filed on Mar. 28, 2018.
(60) Provisional application No. 62/656,551, filed on Apr. 12, 2018.

(51) Int. Cl. C12N 15/10; C12Q 1/6806; C12Q 1/686

(52) U.S. Cl. CPC C12N 15/1079; C12N 15/1096; C12Q 1/6806; C12Q 1/686; C12Q 2563/185; C12Q 2525/143; C12Q 2525/191; C12Q 2535/00; C12N 2310/16

(57) ABSTRACT

Processes and kits are provided for producing sequence specific fragments of nucleic acid molecules, whether from a genome or transcriptome, where one end of the molecule is highly diverse and/or the full-length molecule, whether a gene or a mRNA, is too long for it to be sequenced using currently available sequencing methods. Methods of preparing a sequencing library configured for 5′ or 3′ anchored sequencing, wherein the opposing termini of the library molecules are differentially truncated, and methods of parallel sequencing such libraries are described.

### CROSS REFERENCE TO RELATED APPLICATIONS

This application is a continuation of International Appln. No. PCT/US2019/024623, filed Mar. 28, 2019; which claims the benefit of US Provisional Appln. Nos. 62/649,482 filed Mar. 28, 2018; and 62/656,551, filed Apr. 12, 2018, herein incorporated by reference in their entirety.

### SEQUENCE LISTING

The instant application contains a Sequence Listing which has been submitted electronically in ASCII format and is hereby incorporated by reference in its entirety. Said ASCII copy, created on Apr. 19, 2019, is named 13691-707_600_SL.txt and is 35,388 bytes in size.

### BACKGROUND OF THE DISCLOSURE

The advent of single cell genome amplification techniques and next generation sequencing methods have led to breakthroughs in our ability to sequence the genome and transcriptome of individual biological cells. Despite these advances, it has remained extremely difficult—and often impossible—to obtain the sequence of a gene or transcript having variable length or unknown sequence at a 5′ or 3′ end. Additionally, it is desirable to be able to multiplex samples for efficiency in the sequencing experiments. Methods are described herein to provide for Single End Random Fragmentation sequencing. As described further herein, the ability to decipher barcodes, such as DNA barcodes, within a microfluidic environment can enable linkage of genomic and transcriptomic data with the cell of origin and its phenotype.

### SUMMARY OF THE DISCLOSURE

In a first aspect, a method is provided for preparing a nucleic acid library for sequencing, the method including; obtaining nucleic acid containing RNA from a biological cell; synthesizing nucleic acid (e.g., complementary nucleic acid) from one or more of the RNA nucleic acids; amplifying the synthesized (e.g., complementary) nucleic acids; fragmenting or tagmenting the amplified nucleic acids, which thereby provide a plurality of differentially truncated nucleic acids; amplifying and adding adapters to the plurality of differentially truncated nucleic acids, thereby providing a library of DNA for 5′ or 3′ anchored sequencing, where the DNA library includes a plurality of differentially truncated DNA sample sequences. In various embodiments, the plurality of differentially truncated DNA sample sequences may each further include a barcode (e.g., the barcode is unique for each biological cell). In some embodiments, the barcode may have a sequence of any one of SEQ ID NOS. 1-96.

In various embodiments, the DNA library may include a plurality of differentially 5′ truncated DNA sample sequences, each having the same 3′ sequence as the other differentially 5′ truncated DNA sample sequences of the plurality.

In other embodiments, the DNA library may include a plurality of differentially 3′ truncated DNA sample sequences, each having the same 5′ sequence as the other differentially 3′ truncated DNA sample sequences of the plurality.

In another aspect, a method is provided for sequencing a nucleic acid library, the method comprising: sequencing a DNA library including a plurality of differentially truncated DNA sample sequences (which may be prepared as described anywhere herein); tiling read sequences corresponding to at least one RNA nucleic acid; and reconstructing a full length sequence of the at least one RNA nucleic acid. The DNA library may be a DNA library containing a plurality of differentially 5′ truncated DNA sample sequences each having the same 3′ sequence as the other differentially 5′ truncated DNA sample sequences of the plurality, or it may be a DNA library containing a plurality of differentially 3′ truncated DNA sample sequences each having the same 5′ sequence as the other differentially 3′ truncated DNA sample sequences of the plurality.

In yet another aspect, a method is provided for preparing a nucleic acid library for sequencing, the method including: obtaining nucleic acid containing mRNA molecules from a biological cell; synthesizing cDNA from one or more of the mRNA molecules; amplifying the cDNA, thereby providing amplified DNA molecules, where each of the amplified DNA molecules includes a first portion having a 5′ terminus and a first priming sequence proximal to the 5′ terminus, a third portion containing the 3′ terminus and a second priming sequence proximal to the 3′ terminus, and a second portion comprising a sequence of interest corresponding to a cDNA sequence, wherein the second portion is disposed between the 3′ end of the first portion and the 5′ end of the third portion, wherein the second portion comprises a 5′ region having an unknown nucleic acid sequence and a 3′ region having a known nucleic acid sequence; and tagmenting the amplified DNA molecules, thereby providing a plurality of 5′ truncated DNA molecules, each 5′ truncated DNA molecule of the plurality including a 5′ portion containing a third priming sequence, the third portion of a corresponding amplified DNA molecule, and a second portion consisting of a truncated sequence of interest; wherein the plurality of 5′ truncated DNA molecules is included in the nucleic acid library.

In various embodiments, each of the 5′ truncated DNA molecules may further include a first barcode sequence. In some embodiments, the first barcode sequence may be located between the 3′ end of the second portion of the 5′ truncated DNA molecules and the 5′ end of the third portion of the 5′ truncated DNA molecules. In various embodiments, the first barcode sequence may be unique for mRNA isolated from the biological cell (e.g., the barcode is unique for each biological cell). In some embodiments, the barcode may have a sequence of any one of SEQ ID Nos. 1-96.

In various embodiments, synthesizing the cDNA may be performed with a nested Template Switching Oligonucleotide (TSO).

In various embodiments, tagmenting may further include inserting an adapter, thereby providing the 5′ third priming sequence. In some embodiments, tagmenting may further include inserting a second barcode, wherein the second barcode is disposed 3′ to the third priming sequence and 5′ to the truncated sequence of interest.

In various embodiments, the method may further include amplifying the 5′ truncated DNA molecules. In some embodiments, amplification of the 5′ truncated DNA molecules may be performed with a gene specific 3′ primer. In some embodiments, the gene specific 3′ primer may prime the 5′ truncated DNA molecules at a location within the second portion, at a known gene specific sequence, thus providing a 3′ anchoring point for amplification. In some embodiments, the 3′ anchoring point for amplification may be at a location other than a 3′ terminus of the known nucleic acid sequence of the cDNA sequence.

In various embodiments, the amplification of the 5′ truncated DNA molecules may add a fourth priming sequence to the third portion, and the third and the fourth priming sequences may include adapter sequences configured for parallel sequencing. In other embodiments, the amplification of the 5′ truncated DNA molecules may replace the third portion with a third portion comprising a fourth priming sequence, and the third and the fourth priming sequences may include adapter sequences configured for parallel sequencing.

In various embodiments of the method, the second portions of the 5′ truncated DNA molecules may range in length, containing randomly less than a full-length of the 5′ region having the unknown nucleic acid sequence.

In various embodiments, the nucleic acid library may include a gene specific library. In some embodiments, the nucleic acid library may include a library encoding a TCR or BCR sequence. In some embodiments, the TCR or BCR library may include both heavy and light chain sequences.

In various embodiments, obtaining the mRNA molecules may include capturing mRNA with a capture oligonucleotide having a 3′ terminal dTVI oligonucleotide sequence. In some embodiments, obtaining the mRNA molecules may include capturing the mRNA molecules to a capture object.

In some other embodiments, capturing the mRNA molecules to the capture object may be performed at a location disposed within a microfluidic device. In some embodiments, the location at which the mRNA molecules are captured to the capture object may be an isolation region of a sequestration pen.

In yet another aspect, a method of sequencing a nucleic acid library is provided, the method including: sequencing a nucleic acid library comprising 5′ truncated DNA molecules (e.g., provided by any of the methods having a process including tagmenting amplified DNA molecules); tiling read sequences corresponding to at least one mRNA molecule; and reconstituting a full length sequence of the at least one mRNA molecule. In some embodiments, the at least one mRNA molecule may include a TCR or BCR oligonucleotide sequence. In some embodiments, the TCR or BCR oligonucleotide sequence may be a heavy chain or a light chain oligonucleotide sequence. In various embodiments, the read sequences are about 75 bp in length. In some embodiments, the nucleic acid library comprises 5′ truncated DNA molecules that each further include a barcode. In some embodiments, the barcode may have a sequence of any one of SEQ ID Nos. 1-96.

In a further aspect, a method is provided for preparing a nucleic acid library for sequencing, the method including: obtaining nucleic acid comprising mRNA molecules from a biological cell; synthesizing cDNA from one or more of the mRNA molecules; amplifying the cDNA to produce amplified DNA molecules, where each of the amplified DNA molecules includes a first portion having a 5′ terminus and a RNA polymerase promoter sequence proximal to the 5′ terminus, a third portion comprising a 3′ terminus and a priming sequence proximal to the 3′ terminus, and a second portion corresponding to a cDNA sequence, where the second portion is disposed between the 3′ end of the first portion and the 5′ end of the third portion, and wherein the cDNA sequence of the second portion includes a 5′ region having an unknown nucleic acid sequence and a 3′ region having a known nucleic acid sequence; transcribing the amplified DNA molecules to provide transcribed RNA molecules, each transcribed RNA molecule including a sequence of interest consisting of a copy of the second portion of a corresponding amplified DNA molecule, and a sequence consisting of a copy of the third portion of the corresponding amplified DNA molecule; fragmenting a portion of the transcribed RNA molecules, thereby providing a plurality of 5′ truncated RNA molecules, each truncated RNA molecule of the plurality containing a 5′ portion consisting of a truncated sequence of interest and a 3′ portion including the 3′ priming sequence; and reverse transcribing the plurality of 5′ truncated RNA molecules, thereby providing a plurality of library DNA molecules, each library DNA molecule including a 5′ terminus that includes a second priming sequence, a 3′ terminus that includes the 3′ priming sequence, and a sequence disposed between the 5′ terminus and the 3′ terminus corresponding to the truncated sequence of interest.

In various embodiments, the 5′ portion of each of the plurality of 5′ truncated RNA molecules may include a 5′ region having an unknown nucleic acid sequence and a 3′ region having at least a portion of a known nucleic acid sequence. In some embodiments, the 5′ region of each 5′ truncated RNA molecule may be truncated at the 5′ end of the unknown sequence (i.e., the 5′ end of the second portion of the corresponding amplified DNA molecule).

In various embodiments, each of the amplified DNA molecules may further include a barcode sequence. In some embodiments, the barcode sequence may be located between the 3′ end of the second portion and the 5′ end of the third portion of each amplified DNA molecule. In some embodiments, the barcode may be unique for the mRNA molecule isolated from the biological cell (e.g., the barcode is unique for each biological cell). In some embodiments, the barcode may have a sequence of any one of SEQ ID Nos. 1-96.

In various embodiments, the 3′ region of the second portion of the amplified DNA molecules may be shorter than a complete known DNA sequence for a gene specific DNA product of the mRNA. In some embodiments, each library DNA molecule of the plurality may include the same portion of the known 3′ region of the cDNA.

In various embodiments, synthesizing the cDNA may include reverse transcribing the mRNA molecules. In some embodiments, synthesizing the cDNA may include using a gene-specific primer. In some embodiments, synthesizing the cDNA may include using a nested Template Switching Oligonucleotide.

In various embodiments, amplifying the cDNA may include amplifying with a gene specific 3′ primer. In some embodiments, the gene specific primer may prime the cDNA at a location corresponding to a known gene specific sequence, thus providing a 3′ anchoring point for amplification.

In various embodiments, transcribing the amplified DNA may be performed using a RNA polymerase. In some embodiments, fragmenting the transcribed RNA molecules may include chemically fragmenting the transcribed RNA.

In various embodiments, reverse transcribing the plurality of 5′ truncated RNA molecules may further include inserting an adaptor and thereby providing the second priming sequence. In some embodiments, inserting the adaptor comprises performing PCR subsequent to reverse transcribing the plurality of 5′ truncated RNA molecules. In some embodiments, performing PCR subsequent to reverse transcribing the plurality of 5′ truncated RNA molecules may further include adding sequencing indices to the 5′ and the 3′ termini of the amplified molecules.

In various embodiments, the priming sequence and the second priming sequence may include adapter sequences configured for parallel sequencing.

In some embodiments, reverse transcribing the plurality of 5′ truncated RNA molecules may further include reverse transcribing a second portion of the transcribed RNA molecules, where the second portion of the transcribed RNA molecules has not been fragmented.

In various embodiments, each library DNA molecule of the plurality may include a 5′ truncated region of unknown sequence, where the 5′ truncated region may range in length (e.g., randomly less than a full length of the 5′ region of unknown nucleic acid sequence from the corresponding cDNA). In some embodiments, the plurality of library DNA molecules may include a gene specific library of DNA molecules. In various embodiments, the plurality of library DNA molecules may include a library of DNA molecules encoding a TCR or BCR sequence. In some embodiments, the TCR or BCR DNA library may include both heavy and light chain sequences.

In various embodiments, obtaining the mRNA molecules may include capturing an mRNA molecule with a capture oligonucleotide having a 3′ terminal dTVI oligonucleotide sequence. In some embodiments, obtaining the mRNA molecules may further include capturing the mRNA molecules to a capture object.

In some other embodiments, capturing the mRNA molecules to the capture object may be performed at a location disposed within a microfluidic device. In some embodiments, the location at which the mRNA molecules are captured to the capture object may be an isolation region of a sequestration pen.

In yet another aspect, a method is provided for sequencing a nucleic acid library, the method comprising: sequencing a DNA library (e.g., a DNA library provided by any of the methods in which transcribed RNA molecules are fragmented to produce a plurality of 5′ truncated RNA molecules); tiling read sequences corresponding to at least one mRNA molecule; and reconstructing a full length sequence of the at least one mRNA molecule. In some embodiments, the full-length sequence of the at least one mRNA molecule may include a TCR or BCR oligonucleotide sequence. In other embodiments, the TCR or BCR oligonucleotide sequence may be a heavy chain or a light chain oligonucleotide sequence. In some embodiments, the read sequences may be about 75 bp in length. In some embodiments, each nucleic acid molecule of the DNA library may further include a barcode (e.g., the barcode is unique for nucleic acid originating from each biological cell). In some embodiments, the barcode may have a sequence of any one of SEQ ID Nos. 1-96.

In a further aspect, a method is provided for preparing a nucleic acid library for sequencing, the method including: obtaining nucleic acid containing mRNA molecules from a biological cell; synthesizing cDNA from one or more of the mRNA molecules; amplifying the cDNA to produce amplified DNA molecules, where each of the amplified DNA molecules includes a first portion having a 5′ terminus and a first priming sequence proximal to the 5′ terminus, a third portion including a 3′ terminus and a second priming sequence proximal to the 3′ terminus, and a second portion comprising a copy of a cDNA sequence, wherein the second portion is located 3′ to the first portion and 5′ to the third portion; amplifying the amplified DNA molecules, to insert a specialized priming sequence into a bottom strand, the specialized priming sequence having a third priming sequence linked via a linker containing at least one non-nucleotide moiety to a fourth priming sequence, thereby forming linker-modified amplified DNA molecules; digesting a top strand of the linker-modified amplified DNA molecules, thereby producing a single-strand (“bottom” strand) linker-modified DNA molecule, wherein the single-stranded linker-modified DNA molecule comprises a first portion having a 5′ terminus, wherein the third priming sequence is at (or proximal to) the 5′ terminus and remains linked via the linker containing at least one non-nucleotide moiety to the fourth priming sequence, a third portion having a 3′ terminus and comprising a complement to the first priming sequence, and a second portion comprising a sequence of interest corresponding to a cDNA sequence, wherein the second portion is disposed between the 3′ end of the first portion and the 5′ end of the third portion, and wherein the second portion comprises a complement to the 5′ region having an unknown nucleic acid sequence and a complement to the 3′ region having a known nucleic acid sequence; fragmenting at least a first portion of the single-strand DNA molecules, thereby providing a plurality of fragmented DNA molecules, each fragmented DNA molecule comprising a first portion having a 5′ terminus, wherein the third priming sequence is at (or proximal to) the 5′ terminus and remains linked via the linker containing at least one non-nucleotide moiety to the fourth priming sequence, and a second portion comprising a truncated sequence of interest; circularizing each of the plurality of fragmented DNA molecules, to provide a plurality of circularized DNA molecules, each comprising the truncated sequence of interest and the specialized primer, wherein the third priming sequence remains linked via the linker containing at least one non-nucleotide moiety to the fourth priming sequence; amplifying the plurality of circularized DNA molecules, wherein the fourth priming sequence comprises a binding site for a reverse primer sequence and the third priming sequence comprises a forward primer sequence, thereby providing a plurality of 5′ truncated DNA library molecules, each 5′ truncated DNA library molecule comprising a first portion comprising a 5′ terminus and the fourth priming sequence, a third portion including the third priming sequence, and a second portion including one of the truncated sequences of interest.

In various embodiments, each of the amplified DNA molecules may further include a barcode sequence. In some embodiments, the barcode sequence may be located between the 3′ end of the second portion of the amplified DNA molecule and the 5′ end of the third portion of the amplified DNA molecule. In some embodiments, the barcode is unique for mRNA molecules isolated from the biological cell (e.g., the barcode is unique for each biological cell). In some embodiments, the barcode may have a sequence of any one of SEQ ID Nos. 1-96.

In various embodiments of the method, amplifying the cDNA to provide amplified DNA molecules may be performed using a nested Template Switching Oligonucleotide (TSO). In some embodiments, amplifying the cDNA to provide amplified DNA molecules may be performed with a gene specific 3′ primer. In some embodiments, the gene specific primer may prime the cDNA at a location within a known gene specific sequence, thus providing a 3′ anchoring point for amplification. In various embodiments, the 3′ anchoring point for amplification may be at a location other than a 3′ terminus of the known sequence of the cDNA. In various embodiments of the method, the third and the fourth priming sequences may include adapter sequences configured for parallel sequencing.

In various embodiments, fragmenting comprises enzymatically fragmenting the amplified DNA molecules. In some embodiments, the 5′ truncated DNA molecules may range in length, randomly less than a full length of the 5′ region having the unknown nucleic acid sequence. In some embodiments, each 5′ truncated DNA library molecule of the plurality may include the same 3′ region having the known nucleic acid sequence.

In various embodiments, the plurality of 5′ truncated DNA library molecules may include a gene specific 5′ truncated DNA library. In some embodiments, the plurality of 5′ truncated DNA library molecules may include a 5′ truncated DNA library encoding a TCR or BCR sequence. In various embodiments, the TCR or BCR 5′ truncated DNA library may include both heavy and light chain sequences.

In various embodiments, obtaining the mRNA molecules may include capturing mRNA molecules with a capture oligonucleotide having a 3′ terminal TₙVI oligonucleotide sequence. In some embodiments, obtaining the mRNA molecules may include capturing the mRNA molecules to a capture object. In some embodiments, capturing the mRNA molecules to the capture object may be performed at a location disposed within a microfluidic device. In some embodiments, the location at which the mRNA molecules are captured to the capture object may be an isolation region of a sequestration pen.

In yet another aspect, a method is provided for sequencing a nucleic acid library, the method including: sequencing a plurality of 5′ truncated DNA molecules (e.g., 5′ truncated DNA molecules provided by any of the methods that include circularizing 5′ truncated DNA molecules); tiling read sequences corresponding to at least one mRNA molecule; and reconstituting a full length sequence of the at least one mRNA molecule. In some embodiments, the at least one mRNA molecule may include a TCR or BCR oligonucleotide sequence. In some embodiments, the TCR or BCR oligonucleotide sequence may be a heavy chain or a light chain oligonucleotide sequence. In various embodiments, the read sequences may be about 150 bp in length. In some embodiments, each oligonucleotide of the nucleic acid library may include a barcode. In some embodiments, the barcode may have a sequence of any one of SEQ ID Nos. 1-96.

In another aspect, a kit is provided for preparing a nucleic acid library, the kit including: a RNA capture oligonucleotide; a gene specific primer; and a fragmenting reagent. The RNA capture oligonucleotide may be any RNA capture oligonucleotide described herein. In some embodiments the RNA capture oligonucleotide may have a dTVI sequence at its 3′ terminus. In some embodiments, the RNA capture oligonucleotide may include a priming sequence at or proximal to a 5′ terminus.

In various embodiments of the kit, the gene specific primer may be specific for a TCR or a BCR sequence. In some embodiments, the TCR or BCR gene specific primer may prime both heavy and light chain sequences of the TCR or BCR gene.

In various embodiments of the kit, the fragmenting reagent is a chemical fragmentation reagent or an enzymatic fragmentation reagent. The chemical fragmentation reagent may be any suitable chemical fragmentation reagent as is known in the art, and may include a divalent cation. In some embodiments, the divalent cation may be magnesium and/or zinc. When the fragmenting reagent is an enzymatic fragmentation reagent, the enzymatic fragmentation reagent may include a non-specific nuclease, a restriction endonuclease, or a tagmentation reagent comprising a transposase. Any suitable non-specific nuclease may be used for this process, and in some embodiments, the non-specific nuclease may be DNase 1.

In various embodiments of the kit, the kit may include a reverse transcriptase. In yet other embodiments, the kit may include sets of primers for use in the methods, which may be any primer described herein or may be any other suitable primer for any of the processes. The primers may further include a barcode. In some embodiments, the barcode may have a sequence of any of SEQ ID NOS. 1-96.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A-2B illustrate a microfluidic device having sequestration pens according to some embodiments of the disclosure.

FIG. 2C illustrates a sequestration pen of a microfluidic device according to some embodiments of the disclosure.

FIG. 2D illustrates a coated surface of a microfluidic device according to an embodiment of the disclosure.

FIGS. 2E-2F illustrate electrokinetic features of a microfluidic device according to some embodiments of the disclosure.

FIG. 3 illustrates a sequestration pen of a microfluidic device according to some embodiments of the disclosure.

FIG. 4 illustrates a sequestration pen of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIGS. 6A-6B are schematic representations of embodiments of 5′ truncated, 3′ anchored nucleic acid sequencing library preparation according to some embodiments of the disclosure.

FIG. 7 is a schematic representation of an embodiment of 5′ truncated, 3′ anchored nucleic acid sequencing library preparation according to some embodiments of the disclosure.

FIG. 8 shows photographic and schematic representations of intermediates and end products of 5′ truncated, 3′ anchored nucleic acid sequencing library preparation according to some embodiments of the disclosure.

FIGS. 9A-9C show photographic and graphical representations of intermediates and end products of 5′ truncated, 3′ anchored nucleic acid sequencing library preparation according to some embodiments of the disclosure.

FIGS. 10A-10C are schematic representations of another embodiment of 5′ truncated, 3′ anchored nucleic acid sequencing library preparation according to some embodiments of the disclosure.

FIGS. 11A-11C are schematic representations of another embodiment of 5′ truncated, 3′ anchored nucleic acid sequencing library preparation according to some embodiments of the disclosure.

### DETAILED DESCRIPTION OF THE INVENTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to an x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include or be a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” a “nanofluidic chip”, or a “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the microfluidic device.

As used herein, a “flow path” refers to one or more fluidically connected circuit elements (e.g. channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” constitutes confining a micro-object to a defined area within the microfluidic device.

As used herein, an “isolation structure” and an “isolation region” may refer to structures and regions within a microfluidic device that facilitate confining of a micro-object.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a microfluidic device is an example of a swept region while an isolation region (described in further detail below) of a microfluidic device is an example of an unswept region.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by differential absorbance of light by objects in the field of view.

As used herein, the “clear aperture” of a lens (or lens assembly) is the diameter or size of the portion of the lens (or lens assembly) that can be used for its intended purpose. In some instances, the clear aperture can be substantially equal to the physical diameter of the lens (or lens assembly). However, owing to manufacturing constraints, it can be difficult to produce a clear aperture equal to the actual physical diameter of the lens (or lens assembly).

As used herein, the term “active area” refers to the portion of an image sensor or structured light modulator that can be used, respectively, to image or provide structured light to a field of view in a particular optical apparatus. The active area is subject to constraints of the light path through the optical apparatus, such as the aperture stop of the light path. Although the active area is two-dimensional, it is typically represented as the length of a diagonal line through opposing corners of a square having the same area.

As used herein, an “image light beam” is an electromagnetic wave that is reflected or emitted from a device surface, a micro-object, or a fluidic medium that is being viewed by an optical apparatus. The device can be any microfluidic device as described herein. The micro-object and the fluidic medium can be located within such a microfluidic device.

As used herein, the term “micro-object” refers generally to any microscopic object that may be isolated and/or manipulated in accordance with the present disclosure. Non-limiting examples of micro-objects include: inanimate micro-objects such as microparticles; beads (e.g., polystyrene beads, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, nucleic acids (e.g., oligonucleotides), proteins, carbohydrates, antigens, small molecule signaling moieties, or other chemical/biological species capable of use in an assay. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include: eukaryotic cells, plant cells, animal cells, such as mammalian cells, reptilian cells, avian cells, fish cells, or the like; prokaryotic cells, bacterial cells, fungal cells, protozoan cells, or the like; cells dissociated from a tissue, such as muscle, cartilage, fat, skin, liver, or lung cells, neurons, glial cells, and the like; immunological cells, such as T cells, B cells, plasma cells, natural killer cells, macrophages, and the like; embryos (e.g., zygotes), germ cells, such as oocytes, ova, and sperm cells, and the like; fusion cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a pig, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the terms “maintaining a cell” and “maintaining cells” refer to providing an environment comprising both fluidic and gaseous components and, optionally a surface, that provides the conditions necessary to keep the cell(s) viable and/or expanding.

As used herein, the term “expanding” when referring to cells, refers to increasing in cell number.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein, “antibody” refers to an immunoglobulin (Ig) and includes both polyclonal and monoclonal antibodies; multichain antibodies, such as IgG, IgM, IgA, IgE, and IgD antibodies; single chain antibodies, such as camelid antibodies; mammalian antibodies, including primate antibodies (e.g., human), rodent antibodies (e.g., mouse, rat, guinea pig, hamster, and the like), lagomorph antibodies (e.g., rabbit), ungulate antibodies (e.g., cow, pig, horse, donkey, camel, and the like), and canidae antibodies (e.g., dog); primatized (e.g., humanized) antibodies; chimeric antibodies, such as mouse-human, mouse-primate antibodies, or the like; and may be an intact molecule or a fragment thereof (such as a light chain variable region (VL), heavy chain variable region (VH), scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen. In some embodiments, antibody fragments may be derivatized to exhibit structural features that facilitate clearance and uptake, e.g., by the incorporation of galactose residues.

An antigen, as referred to herein, is a molecule or portion thereof that can bind with specificity to another molecule, such as an Ag-specific receptor. Antigens may be capable of inducing an immune response within an organism, such as a mammal (e.g., a human, mouse, rat, rabbit, etc.), although the antigen may be insufficient to induce such an immune response by itself. An antigen may be any portion of a molecule, such as a conformational epitope or a linear molecular fragment, and often can be recognized by highly variable antigen receptors (B-cell receptor or T-cell receptor) of the adaptive immune system. An antigen may include a peptide, polysaccharide, or lipid. An antigen may be characterized by its ability to bind to an antibody's variable Fab region. Different antibodies have the potential to discriminate among different epitopes present on the antigen surface, the structure of which may be modulated by the presence of a hapten, which may be a small molecule.

In some embodiments, an antigen is a cancer cell-associated antigen. The cancer cell-associated antigen can be simple or complex; the antigen can be an epitope on a protein, a carbohydrate group or chain, a biological or chemical agent other than a protein or carbohydrate, or any combination thereof; the epitope may be linear or conformational.

The cancer cell-associated antigen can be an antigen that uniquely identifies cancer cells (e.g., one or more particular types of cancer cells) or is upregulated on cancer cells as compared to its expression on normal cells. Typically, the cancer cell-associated antigen is present on the surface of the cancer cell, thus ensuring that it can be recognized by an antibody. The antigen can be associated with any type of cancer cell, including any type of cancer cell that can be found in a tumor known in the art or described herein. In particular, the antigen can be associated with lung cancer, breast cancer, melanoma, and the like. As used herein, the term “associated with a cancer cells,” when used in reference to an antigen, means that the antigen is produced directly by the cancer cell or results from an interaction between the cancer cell and normal cells.

As used herein, “B” used to denote a single nucleotide, is a nucleotide selected from G (guanosine), C (cytidine) and T (thymidine) nucleotides but does not include A (adenine).

As used herein, “H” used to denote a single nucleotide, is a nucleotide selected from A, C and T, but does not include G.

As used herein, “D” used to denote a single nucleotide, is a nucleotide selected from A, G, and T, but does not include C.

As used herein, “K” used to denote a single nucleotide, is a nucleotide selected from G and T.

As used herein, “M” used to denote a single nucleotide, is a nucleotide selected from A or C.

As used herein, “N” used to denote a single nucleotide, is a nucleotide selected from A, C, G, and T.

As used herein, “R” used to denote a single nucleotide, is a nucleotide selected from A and G.

As used herein, “S” used to denote a single nucleotide, is a nucleotide selected from G and C.

As used herein, “V” used to denote a single nucleotide, is a nucleotide selected from A, G, and C, and does not include T.

As used herein, “Y” used to denote a single nucleotide, is a nucleotide selected from C and T.

As used herein, “I” used to denote a single nucleotide is inosine.

As used herein, A, C, T, G followed by “*” indicates phosphorothioate substitution in the phosphate linkage of that nucleotide.

As used herein, IsoG is isoguanosine; IsoC is isocytidine; IsodG is an isoguanosine deoxyribonucleotide and IsodC is a isocytidine deoxyribonucleotide. Each of the isoguanosine and isocytidine ribo- or deoxyribo-nucleotides contain a nucleobase that is isomeric to guanine nucleobase or cytosine nucleobase, respectively, usually incorporated within RNA or DNA.

As used herein, rG denotes a ribonucleotide included within a nucleic acid otherwise containing deoxyribonucleotides. A nucleic acid containing all ribonucleotides may not include labeling to indicate that each nucleotide is a ribonucleotide, but is made clear by context.

As used herein, a “priming sequence” is an oligonucleotide sequence which can be part of a larger oligonucleotide but, when separated from the larger oligonucleotide such that the priming sequence includes a free 3′ end, can function as a primer in a DNA (or RNA) polymerization reaction.

The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

Single End Random Fragment Sequencing (SERF Seq).

Currently, it is difficult to provide robust sequencing results from Next Generation Sequencing (NGS) sequencing platforms under several different circumstances, such as long sequences (e.g., a gene has a sequence of greater than about 500 bp), as most current NGS sequencers start to deteriorate in quality after 500 bp. Another scenario where NGS sequencing becomes difficult is where one end of the sequence is highly diverse.

Single End Random Fragment sequencing (SERF seq) is a set of novel methods designed to sequence specific fragments of genome or transcriptome where one end of the sequence is highly diverse and/or the gene is large. As referred to herein, a region of a gene sequence that is highly diverse, large, or novel, is referred to as an “unknown region.” Thus, an unknown sequence can be a sequence that has never been sequenced before, or it can be a sequence that has been sequenced before but it is nevertheless unknown in the sense that it exhibits variation, either in its sequence (e.g., it may contain a region of hypervariable sequence) or with regard to another sequence to which it is juxtaposed, whether by genetic recombination, alternative splicing, or the like. When the unknown sequence is juxtaposed with another sequence which is known, the other sequence can be referred to herein as a “known region” or “known nucleic acid sequence.” As used herein in reference to a nucleic acid sequence, such as a gene, a “large” sequence is at least 400 bp or longer (e.g., at least 450, 500, 550, 600, 650, 700, 750, 800, 900, 1000, 1100, 1200, 1300, 1400, 1500 bp, or longer).

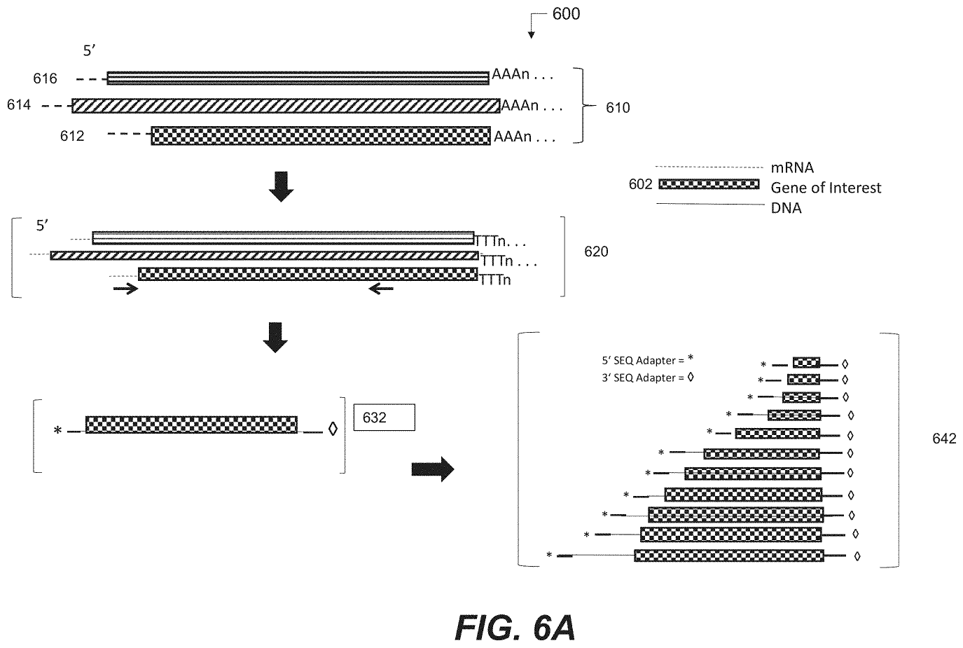

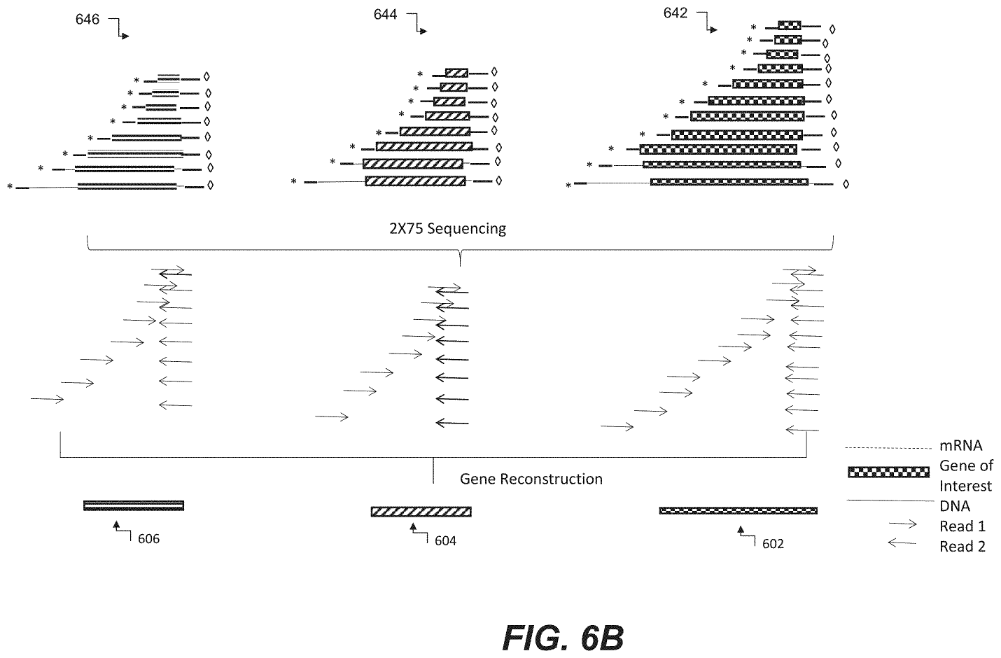

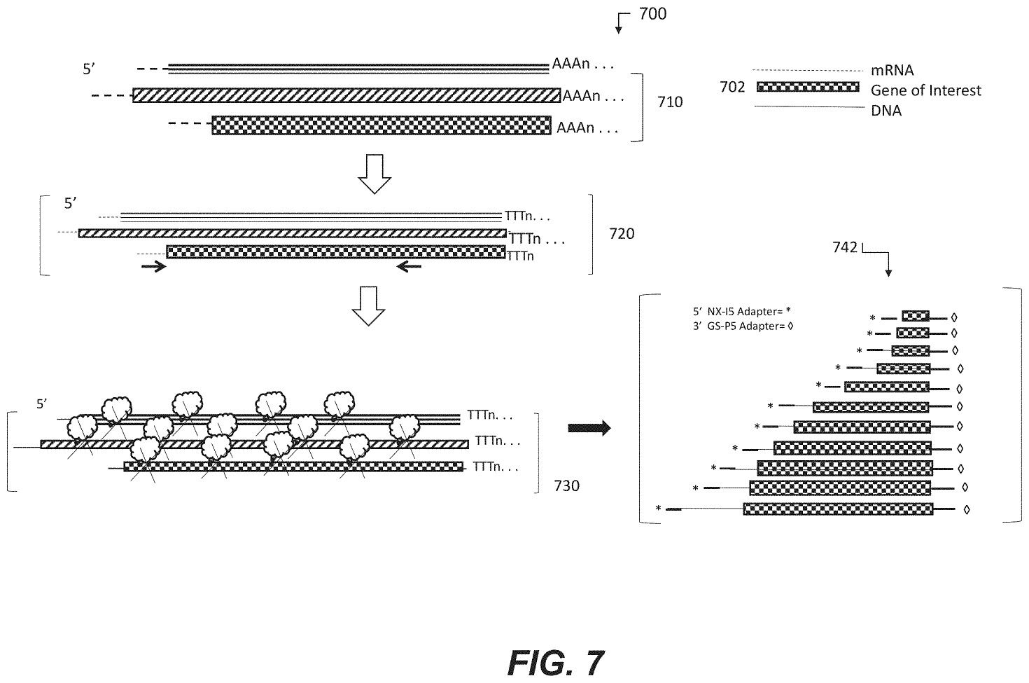

As shown schematically in FIGS. 6A-6B and 7, the disclosed methods provide for the capture/generation of amplicon(s) of interest from a sample of fragmented mRNA, fragmented DNA, or tagmented DNA by anchoring on to a portion of a known end of a gene, such as shown in reaction complex 620. This gives the ability to sequence genes that undergo complex gene recombination events including, but not limited to B-cell receptors genes, T-cell receptors genes, MHC complex genes, alternatively spliced genes, and genes that have undergone gene editing. This method also provides the ability to sequence genes having variable lengths (e.g. 200 bp to 2000 bp), while providing effective coverage over the entire range of possible lengths.

Generally, a nucleic acid library suitable for sequencing may be prepared using any of the various methods described in further detail below. Nucleic acid including mRNA 610 may be captured from a biological cell, and nucleic acids 620 may be synthesized from the original template nucleic acid. The synthesized nucleic acids may be amplified, and the amplified nucleic acids, which may be DNA 632, may be fragmented or tagmented. Alternatively, the amplified nucleic acids 632 may be converted into a different class of nucleic acid, such as RNA, which may be fragmented and reverse transcribed to provide fragmented DNA molecules. In either scenario, a plurality of differentially truncated nucleic acids results. The plurality of differentially truncated nucleic acids may be further modified, such as by amplification and insertion of sequencing adapters, priming sequences, index molecules and/or barcodes to provide a DNA library 642 suitably sized and adapted for parallel sequencing.

Depending on a selection of either 3′ anchored amplification or 5′ anchored amplification, a plurality of differentially 5′ truncated DNA molecules, each having the same 3′ sequence, is provided, or a plurality of differentially 3′ truncated DNA molecules, each having the same 5′ sequence, is provided. The plurality of differentially 5′ truncated DNA molecules or the differentially 3′ truncated DNA molecules form a DNA library for sequencing. While the description provided herein is directed primarily to providing differentially 5′ truncated DNA molecules, the methods may be understood to encompass preparing differentially 3′ truncated DNA molecules as a sequencing library by employing the principles described. An additional benefit of using these methods to prepare a sequencing library having differentially 3′ truncated DNA molecules is that a barcode can be included proximal to the 5′ terminus of the RNA capture oligonucleotide, permitting barcoding at a first step of these processes. The remainder of each workflow can be devised to retain the barcode within the final adapted and sized oligonucleotides comprising the nucleic acid sequencing library.

The differentially 5′ truncated DNA library or the differentially 3′ truncated DNA library may be sequenced, using any suitable sequencing method. The resultant read sequences, which can include fragments of at least one RNA nucleic acid captured from the biological cell, may be tiled, and the full-length sequence of the at least one RNA nucleic acid may be reconstructed. As used herein, a “full-length” RNA or mRNA molecule is a molecule that is substantially the same length as the RNA or mRNA molecule that was present in a sample used to make a sequencing library. A full-length RNA or mRNA molecule can be, but need not be, the longest possible version of the RNA or mRNA. Full-length RNA or mRNA molecules can include molecules that lack certain 5′ sequences due to alternative splicing, degradation, or the like.

As shown in FIG. 6B, this method can also provide multiplexing capabilities, combining multiple samples into a single sequencing experiment, while providing the ability to deconvolute the resultant multiplexed sequencing reads back to each respective sample. For example, the differentially 5′ truncated DNA libraries 642, 644, 646 may be constructed, and combined after barcoding. The combined sample may be sequenced and provide differentiable sequence reads, which may be tiled and permit reconstruction of the respective sequences 602, 604, 606 of the RNA molecules 612, 614, 616 initially captured from one or more biological cells.

These methods provide a reduction in reverse transcription and PCR errors compared to amplification and adaptation of whole genes (e.g., long read lengths), where enzyme errors may proliferate. Long reads, such as reads that are greater than 400 bp, e.g., over 500, 600, 700, 800, 900, 1000, 1100, 1200, 1300, 1400, 1500 bp, or more, may be read by using a tagmentation process to obtain suitably sized fragments that can be tiled to assemble the full-length sequence. It can be possible to address amplicons over 2 kb or more, by increasing read convergence and varying fragmentation time to increase tiling and coverage of each region of the gene. The Phred quality score Q30 for a 2×75 bp run for the sequencing for a DNA library resulting from tagmentation of DNA, as described in Experiment 1, below, remained over 94% during 150 cycles (data not shown), whereas a 2×300 run demonstrated a Q30 of about 75% over 600 cycles (data not shown).

Additionally, the sequencing run time is greatly decreased from about 56 hours for a 2×300 sequencing run compared to about 20 hours for the 2×75 fragments provided in this method. Costs are also decreased from about $1530 for the 2×300 run to about $875 for the 2×75 bp run.

The disclosed methods provide higher precision, enabling detection of gene fusion products, and provide high resolution mapping of fusion products. Novel transcripts are more easily identified using this method, and permit sequencing of genes with high percentages of recombination/splicing products or complex patterns of recombination/splicing products.

These methods are particularly suitable for genes encoding for BCR, TCR, ProtoCadherin (cell adhesion proteins), Down Syndrome adhesion molecule (DSCAM), and other Ig superfamily proteins. Additionally, this method may be utilized for sequencing genes that have been modified through gene editing or from organisms that do not have well defined genomes.

While the methods describe the isolation of mRNA in detail, any other type of RNA may be isolated and processed to form sequencing libraries. Other types of RNA include but are not limited to total RNA, small RNA (including microRNA and transfer RNA), ribosomal RNA, and the like. Further, either 5′ anchored libraries or 3′ anchored libraries may be provided, as described herein.

Additionally, these methods may also be applied to genomic DNA library preparation. When preparing nucleic acid libraries from gDNA, the initial reverse transcription step converting RNA to cDNA, as described below for RNA isolated from a biological cell, may be replaced by performing an initial single strand DNA synthesis using appropriate enzymatic or chemical synthesis, as is known in the art. The rest of the methods to prepare a DNA sequencing library using either 3′ anchored amplification or 5′ anchored amplification may be performed similarly as to the processes described herein.

Specific Adapters Used in Sequencing Libraries.

The approaches shown here are adapted for eventual use with Illumina® sequencing by synthesis chemistries, but the methods are not so limited. Any sort of sequencing chemistries may be suitable for use within these methods and may include emulsion PCR, sequencing by synthesis, pyrosequencing and semiconductor detection. One of skill in the art can adapt the methods and construction of the capture oligonucleotides and associated adapters, primers, and the like to use these methods within other massively parallel sequencing platforms and chemistries, such as PacBio long read systems (SMRT, Pacific Biosystems), Ion Torrent (ThermoFisher Scientific), Roche 454, Oxford Nanopore, and the like.

Methods.

For any of the methods described herein, the biological cell may be exported from a microfluidic device or any other kind of cell holder to a well plate, where the biological cell may be present as a single cell within a well of the wellplate or as more than one biological cell present in the well of the wellplate. In cases where there is more than one biological cell, the plurality of biological cells may be a clonal population of cells. The biological cell may be any suitable type of cell (e.g., any of the cell types disclosed herein, including any of the exemplary cell types disclosed in connection with the definition of “cell” provided above), but the method is not limited to the exemplary cell types described herein.

The biological cell may be lysed using any suitable method and reagents to effect lysis of the cell membrane, thereby making RNA molecules available for capture. The RNA molecules may be captured to a capture object such as a bead, which may be paramagnetic or may not be paramagnetic.

In some embodiments, lysis of the biological cell may be performed while the biological cell is disposed within a microfluidic device, and RNA molecules (which may be any kind of RNA, including mRNA) may be captured by a capture object. In some embodiments, the biological cell(s) may be disposed within an isolation region of a sequestration pen as described herein. The capture object bearing captured RNA molecules may be exported from the microfluidic device and processing may continue as for RNA molecules which are originally captured to a capture object in a well plate. In some embodiments, the capture object may have a barcode which may be read on chip and also read from sequencing a portion of cDNA off chip, thereby allowing sequencing data from a well of the well plate to be correlated with the sequestration pen of the microfluidic device from which the capture object was exported.

The microfluidic device, which may house biological cells prior to or during RNA capture, may further include a dielectrophoretic activation substrate, including electrodes which may be activated to provide dielectrophoretic (DEP) forces within the microfluidic environment. The DEP forces may be used to export biological cells from sequestration pens, introduce the biological cells to the sequestration pens, introduce capture object(s) to the sequestration pens, and/or export capture object having captured RNA molecules from the sequestration pens of the microfluidic device.

No matter the manner in which RNA molecules are provided to the well plate for the methods, the RNA molecules are reverse transcribed to provide cDNA. In some embodiments, RNA molecules may be captured/primed with a capture oligonucleotide having a 3′ terminal dTVI oligonucleotide sequence. Capture oligonucleotides having a 3′ terminal dTVI oligonucleotide sequence may advantageously provide more captured RNA molecules compared to a capture oligonucleotide having a 3′ terminal dTVN oligonucleotide sequence, but one may suitably use the 3′ terminal dTVN capture oligonucleotide, adjusting for the differences in product capture. In some embodiments, the capture oligonucleotide may further include a 5′-biotin moiety.

The reverse transcription (RT) reaction also may include a Template Switching Oligonucleotide (TSO), which, optionally may be 5′-biotinylated. The TSO or bio_TSO may further include additional nucleotides to help amplify specific desired amplicons, such as BCR specific amplicons. The TSO or bio_TSO may further be a nested TSO. The product of the RT reaction is a plurality of cDNA molecules, which are used in any of the methods.

For any of the methods, barcodes may be introduced, permitting multiplexing of the sequencing experiments, such as shown in FIG. 6B where libraries 642, 644, and 646 may be combined.

Method Including Tagmentation.

A better understanding of the methods may be had by turning to the figures. FIG. 7 shows a schematic representation of a method 700 for preparing a nucleic acid library, utilizing tagmentation to produce fragmented nucleic acids for a 3′ anchored sequencing library. This method may also be adapted to suitably provide fragmented nucleic acids for a 5′ anchored sequencing library, but for ease of review, the method will be discussed in terms of providing the 3′ anchored sequencing library.

The method of preparing a nucleic acid library for sequencing includes obtaining nucleic acid comprising mRNA molecules 710 from a biological cell, which may be obtained as described above or may be obtained in any suitable fashion. The cDNA 720 is synthesized from one or more of the mRNA molecules, which may be performed as described above or any other suitable fashion. The cDNA may be synthesized using a Template Switching Oligonucleotide (TSO) or a nested TSO.

The cDNA 720 is subsequently amplified, thereby providing amplified DNA molecules, wherein each of the amplified DNA molecules comprises a first portion having a 5′ terminus and a first priming sequence proximal to the 5′ terminus, a third portion comprising the 3′ terminus and a second priming sequence proximal to the 3′ terminus, and a second portion comprising a sequence of interest corresponding to a cDNA sequence, wherein the second portion is disposed between the 3′ end of the first portion and the 5″ end of the third portion, wherein the second portion comprises a 5′ region having an unknown nucleic acid sequence and a 3′ region having a known nucleic acid sequence. In some embodiments, the first priming sequence proximal to the 5′ terminus may be disposed at the 5′ terminus, or there may be one or more nucleotides disposed 5′ to the beginning of the first priming sequence. In some embodiments, the second priming sequence may be disposed such that the last nucleotide of the priming sequence is at the 3′ terminus of the amplified DNA molecules, or there may be one or more nucleotides 3′ to the end of the second priming sequence. In some embodiments, the 5′ region of the second portion having an unknown nucleic acid sequence corresponds to a full-length sequence of an unknown or variable region of a gene. The second portion containing the 5′ region having an unknown nucleic acid sequence and a 3′ region having a known nucleic acid sequence may be a full-length sequence of the RNA molecule captured from the biological cell. In some embodiments the 3′ region of the second portion having a known nucleic acid sequence may include less than all of a known region of the gene. Alternatively, for methods that provide a 3′ anchored sequencing library, the second portion can contain a 3′ region having an unknown sequence and a 5′ region having a known sequence, and the second portion may be a full-length sequence of the RNA molecule captured from the biological cell or a fragment thereof.

The amplified DNA molecules may then be tagmented (reaction complex 730), providing a plurality of 5′ truncated DNA molecules, each truncated DNA molecule of the plurality comprising a 5′ portion comprising a third priming sequence, the third portion of a corresponding amplified DNA molecule, and a second portion consisting of a truncated sequence of interest. The second portions of the 5′ truncated DNA molecules, containing the truncated sequence of interest range in length, randomly less than a full-length of the 5′ region of the captured RNA molecules having the unknown nucleic acid sequence. The plurality of randomly 5′ truncated DNA molecules contain a plurality of sequences of interest for the nucleic acid library. A subsequent amplification of the tagmentation product of complex 730 is performed to provide the plurality of amplicons 742 for sequencing. The amplification inserts an adapter, thereby providing the 5′ third priming sequence. The amplification also inserts a fourth priming sequence to the third portion, and the third and the fourth priming sequences may comprise adapter sequences configured for parallel sequencing. The amplification may also insert a first barcode sequence. The first barcode sequence may be located between the 3′ end of the second portion of the 5′ truncated DNA molecules and the 5′ end of the third portion of the 5′ truncated DNA molecules. The first barcode sequence may be unique for the mRNA molecule isolated from the biological cell.

The amplification of the tagmentation product may further include inserting a second barcode, wherein the second barcode is disposed 3′ to the third priming sequence and 5′ to the truncated sequence of interest.

Amplification of the 5′ truncated DNA molecules may be performed with a gene specific 3′ primer. The amplification products 742 may therefore have only gene specific products, thereby providing a gene specific library.

In some embodiments, the gene specific 3′ primer may prime the 5′ truncated DNA molecules at a location within the second portion, at a known gene specific sequence, thus providing a 3′ anchoring point for amplification. In other embodiments, where a 3′ region of a gene contains variable or complex sequences, a 5′ anchoring point may be chosen, where the amplification of tagmented product will thereby produce a library of 3′ truncated DNA molecules having 5′ regions containing the same, known gene region.

The 3′ anchoring point for amplification may be chosen so that it may not be at a 3′ terminus of a known DNA sequence of the cDNA. The 3′ anchoring point for amplification may be selected to amplify less than the complete known DNA sequence. The term “complete known DNA sequence,” as used herein, refers to a DNA sequence encoding one or more discrete/complete protein domains. Only a small or selected portion of the known region (e.g., constant region of an antibody light chain or heavy chain) may be included within the chosen anchoring point, thereby providing smaller amplicons for sequencing. This may provide fewer errors in sequencing. In some embodiments, only about 10%, 15%, 20%, 25%, 30%, 35%, 40%, 45%, 50%, 55%, 60%, 65%, 70% or about 75% of the known region of the gene may be included by preselection of the 3′ (or 5′) anchoring point.

In some embodiments, the nucleic acid library may be a library encoding a TCR or BCR sequence. In some embodiments, the TCR or BCR library may include both heavy and light chain sequences.

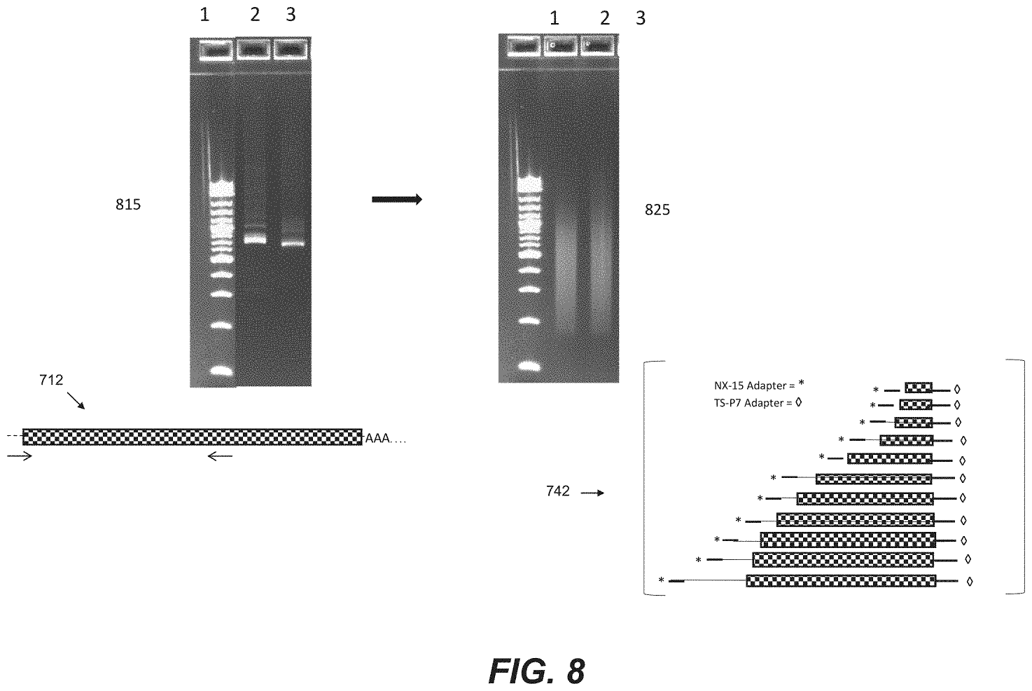

FIG. 8 shows some aspects of the intermediates obtained in this method. A captured RNA molecule 712 was reverse transcribed using a nested TSO, and amplified using a gene specific primer for human Hc sequences (lane 2 of gel 815) and a second amplification using a gene specific primer for human Kc sequences (lane 3 of gel 815), with DNA ladder in lane 1. Each amplified product was individually transformed using the tagmentation and amplification steps described above to provide fragmented and adapted libraries 742 for human Hc (lane 1 of gel 825) and for human Kc (lane 2 of gel 825).

The libraries produced by this method may be sequenced by any suitable method. The read sequences may be tiled to reconstruct the full-length sequence of the RNA molecule captured from the biological cell. The RNA molecule may be any kind of RNA molecule, and some non-limiting examples include a TCR or BCR oligonucleotide sequence. The TCR or BCR oligonucleotide sequence may be a heavy chain or a light chain oligonucleotide sequence. In various embodiments, the read sequences are about 75 bp in length.

A nucleic acid library that is 5′ anchored may also be provided in this method. In the process of amplifying the cDNA produced from the captured RNA (reaction complex 720), a forward primer that starts amplification at a desired location in the 5′ region of a cDNA having a known (e.g., constant) 5′ region and a unknown (e.g., variable) 3′ region may be selected, while the reverse primer may be selected to begin amplification within or proximal to the polyT region of the cDNA. The resulting amplicons can be tagmented similarly to that shown in complex 730, and the resulting amplifications to insert the barcode, indices and sequencing adapters may be selected to introduce the barcode 5′ to the known region of the sample DNA sequences, where the amplicons include a 3′ truncated sample DNA region. This will permit the same coverage of sequencing as in the 5′ anchored libraries.

Method Including Chemical Fragmentation.

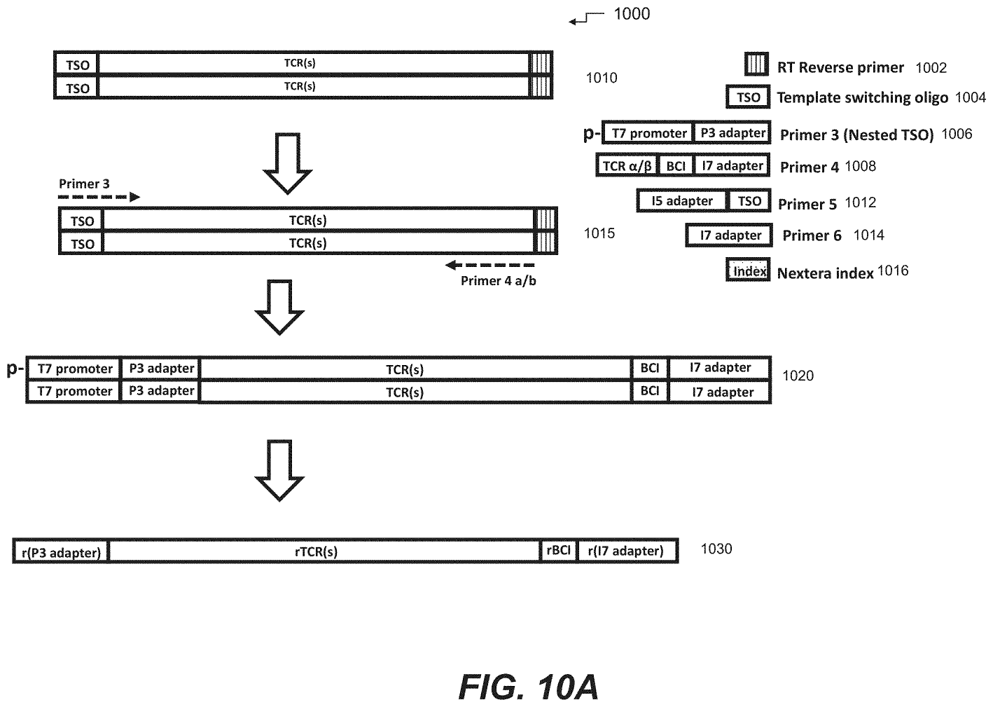

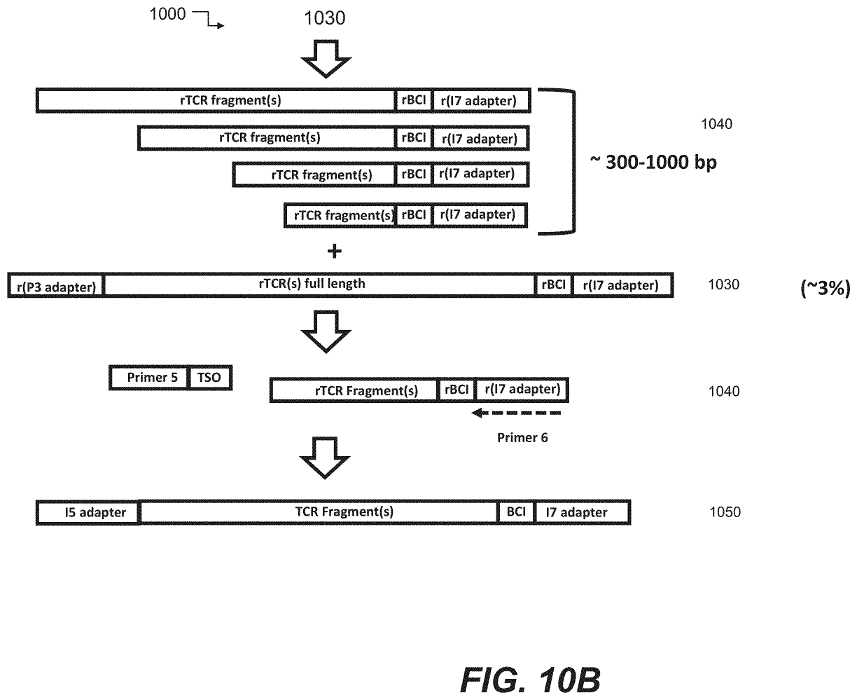

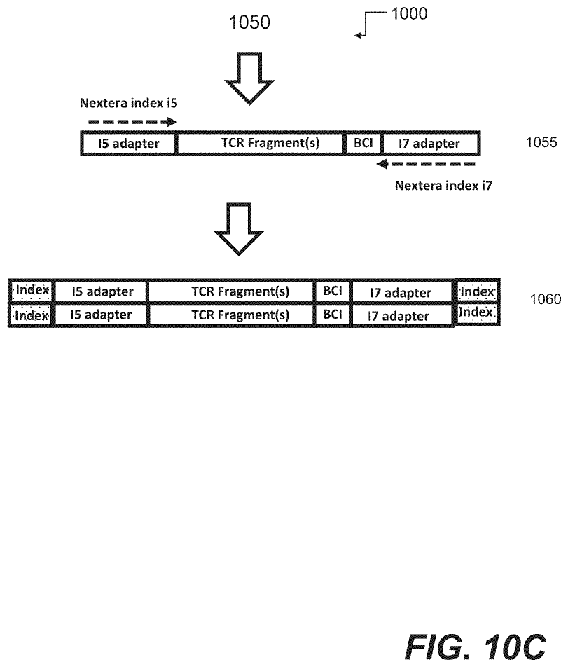

Referring to FIGS. 10A-C, another method 1000 of providing a nucleic acid library for sequencing includes: obtaining nucleic acid comprising mRNA molecules from a biological cell; and synthesizing cDNA from one or more of the mRNA molecules, which may be performed in any suitable manner as described above, and is illustrated here using primers 1002 and 1004. This method will be described in terms of providing a 5′ truncated DNA library, which is useful for sequencing a gene having a variable/complex/recombined 5′ region and a constant or known 3′ region, such as a TCR or BCR gene. However, the method is not so limited and may further be used to provide a DNA library having a plurality of 3′ truncated DNA molecules, by redesigning which fragments will be amplified and adapted.

The cDNA is amplified (reaction complex 1015, which may use a nested TSO such as illustrated in FIG. 10A, primer 1006, along with primer(s) 1008, to produce amplified DNA molecules 1020, where a first portion having a 5′ terminus has had a RNA polymerase promoter sequence introduced proximal to the 5′ terminus. The promoter sequence may start at the 5′ terminus, or there may be one or more nucleotides 5′ to the start of the promoter sequence. The amplified DNA molecules each include a third portion having a 3′ terminus and a priming sequence proximal to the 3′ terminus. The priming sequence of the third portion may be disposed such that the last nucleotide of the priming sequence is disposed at the 3′ terminus of the amplified DNA molecules or there may be one or more nucleotides disposed 3′ to the last nucleotide of the priming sequence of the third portion. The amplified DNA molecules further include a second portion corresponding to a cDNA sequence, wherein the second portion is disposed between the 3′ end of the first portion and the 5′ end of the third portion, and wherein the second portion comprises a 5′ region having an unknown nucleic acid sequence and a 3′ region having a known nucleic acid sequence. The second portion containing the 5′ region having an unknown nucleic acid sequence and a 3′ region having a known nucleic acid sequence may be a full length sequence of the RNA molecule captured from the biological cell. Alternatively, for methods that provide a 3′ anchored sequencing library, the second portion can contain a 3′ region having an unknown sequence and a 5′ region having a known sequence, and the second portion may be a full-length sequence of the RNA molecule captured from the biological cell. Further, amplifying the cDNA may include amplifying with a gene specific 3′ (or 5′) primer, yielding only gene specific amplified DNA molecules. The gene specific primer may prime the cDNA at a location corresponding to a known gene specific sequence, thus providing a 3′ (or 5′) anchoring point for amplification. In some embodiments, the 3′ anchoring point may not be the 3′ terminal nucleotide of the known DNA sequence. The 3′ region of the second portion of the amplified DNA molecules may be selected to be shorter than a complete known DNA sequence for the mRNA. The term “complete known DNA sequence” as used herein refers to a DNA sequence encoding one or more discrete/complete protein domains. Only a small or selected portion of the known region (e.g., constant region of an antibody light chain or heavy chain) may be included within the chosen anchoring point, which provides smaller amplicons for sequencing. This may provide fewer errors in sequencing. In some embodiments, only about 10%, 15%, 20%, 25%, 30%, 35%, 40%, 45%, 50%, 55%, 60%, 65%, 70% or about 75% of the known region of the gene may be included by preselection of the 3′ (or 5′) anchoring point.

Each of the amplified DNA molecules may further include a barcode sequence, and the barcode sequence may be any suitable barcode. In some embodiments, the barcode may have a sequence of any one of SEQ ID NOS. 1-96, as shown in Table 1. In some embodiments, the barcode sequence may be located between the 3′ end of the second portion and the 5′ end of the third portion of each amplified DNA molecule. In some embodiments, the barcode is unique for the RNA molecule (e.g., mRNA) isolated from the biological cell.

The amplified DNA molecules 1020 are transcribed using a RNA polymerase, which may be any suitable RNA polymerase, to provide transcribed RNA molecules 1030, each transcribed RNA molecule including a sequence of interest consisting of a (ribonucleic) copy of the second portion of a corresponding amplified DNA molecule, and a sequence consisting of a (ribonucleic) copy of the third portion of the corresponding amplified DNA molecule. The transcribed RNA molecule may further include ribonucleic copies of the barcodes introduced in the amplification described in the previous paragraph. A “copy” as used herein to refer to the transcribed RNA molecules, includes an “exact” copy (T's of the amplified DNA template molecules are converted to U's in an “exact” ribonucleic copy) or a copy that includes one or more errors, such as may be introduced by an RNA polymerase during RNA transcription.

At least a portion of the transcribed RNA molecules are fragmented, thereby providing a plurality of 5′ truncated RNA molecules 1040, as shown in FIG. 10B, each truncated RNA molecule of the plurality including a 5′ portion consisting of a truncated sequence of interest and a 3′ portion comprising the 3′ priming sequence. The RNA molecules may be chemically fragmented using any appropriate fragmenting buffer as is known in the art. The chemical fragmenting buffer may include a divalent cation, which may be magnesium and/or zinc. The conditions of the chemical fragmenting can be chosen to produce properly sized fragments by changing concentration, time, or temperature to achieve the desired results. The (ribonucleic) copies of the barcode and the 3′ priming sequence in the 3′ portion are retained, and the 3′ priming sequence provides the 3′ anchoring sequence for later amplification. The 5′ portion of each of the plurality of 5′ truncated RNA molecules has a 5′ region having an unknown nucleic acid sequence and a 3′ region having at least a portion of a known nucleic acid sequence (e.g., constant region of a gene). The 5′ region of each 5′ truncated RNA molecule may be truncated at the 5′ end of the unknown sequence (i.e., of the second portion of a corresponding amplified DNA molecule). The plurality of 5′ truncated RNA molecules thus have differentially truncated 5′ regions which correspond to the unknown (variable) region of the RNA molecule captured from the biological cell.

The plurality of 5′ truncated RNA molecules are then reverse transcribed (as shown in reaction complex 1040 using primers 1012 and 1014), providing a plurality of DNA molecules 1050, including a 5′ terminus that includes a second priming sequence, a 3′ terminus that includes the 3′ priming sequence, and a sequence disposed between the 5′ terminus and the 3′ terminus corresponding to a truncated sequence of interest. Reverse transcribing the plurality of 5′ truncated RNA molecules further includes inserting an adaptor and thereby providing the second priming sequence. The priming sequence and the second priming sequence may include adapter sequences configured for parallel sequencing.

Reverse transcribing the plurality of 5′ truncated RNA molecules may further include reverse transcribing a second portion of the transcribed RNA molecules, where the second portion of the transcribed RNA molecules has not been fragmented. The second portion of transcribed RNA molecules may be about 1%, 3%, 5%, 7% or about 9% of the total.

The 3′ terminus including the 3′ priming sequence of the DNA molecules transcribed from the 5′ truncated RNA molecules still retains the barcode (BCI) as shown in DNA molecules 1050. Amplification of DNA molecules 1050 follows as in complex 1055, where the product DNA molecule 1060 have indices 1016 inserted to the 5′ and the 3′ termini for parallel sequencing.

Each library DNA molecule of the plurality 1060 may include a 5′ truncated region of unknown sequence, wherein the 5′ truncated region ranges in length (e.g., randomly less than a length of the full-length unknown sequence of the full cDNA). The method provides a plurality of library DNA molecules 1060, which may be a gene specific library of DNA molecules. The plurality of library DNA molecules may be a library of DNA molecules encoding a TCR or BCR sequence. The TCR or BCR DNA library may include both heavy and light chain sequences.

The libraries produced by this method may be sequenced by any suitable method. The read sequences may be tiled to reconstruct the full-length sequence of the RNA molecule captured from the biological cell. The RNA molecule may be any kind of RNA molecule, and some non-limiting examples include a TCR or BCR oligonucleotide sequence. The TCR or BCR oligonucleotide sequence may be a heavy chain or a light chain oligonucleotide sequence. In various embodiments, the read sequences are about 75 bp in length.

A nucleic acid library that is 5′ anchored may also be provided in this method for a RNA molecule having a known (e.g., constant) 5′ region and an unknown (e.g., variable) 3′ region. In the amplification providing amplified DNA 1020 from cDNA isolated from the RNA, a forward primer may start amplification at a point within the 5′ region (which can be selected to be other than at the 5′ end of the known 5′ region while additionally introducing a barcode just 5′ of that selected point. The forward primer also introduces the T7 phosphorylation promotor and a priming sequence 5′ to the 5′ end of the barcode. The reverse primer is selected to begin amplification within or proximal to the polyT region of the cDNA. The resulting amplicons can then be transcribed to single stranded RNA as described above, and fragmented chemically. The fragmented RNA may be reverse transcribed to DNA, inserting a priming sequence within or proximal to the polyT region of the DNA amplicons arising from the RNA captured from the biological cell. The priming sequence previously introduced at the 5′ terminus may now be used, as in complex 1055 to initiate forward amplification. A library containing 5′ anchored amplicons having indices, barcode and 3′ truncated DNA samples sequences is produced.

Method Including Enzymatic Fragmentation and Circularization.

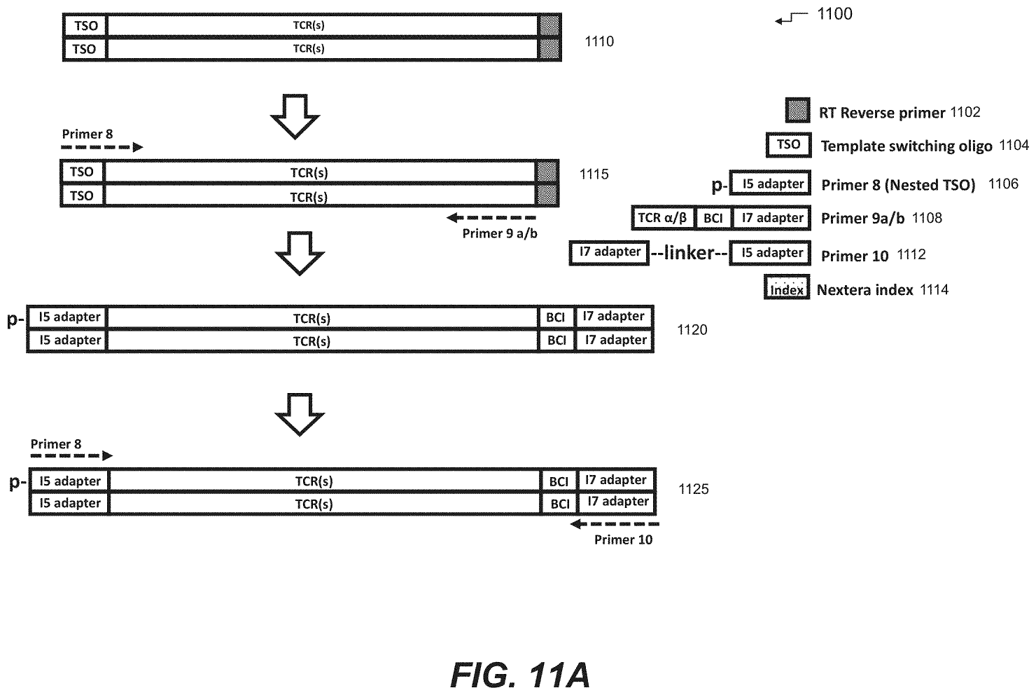

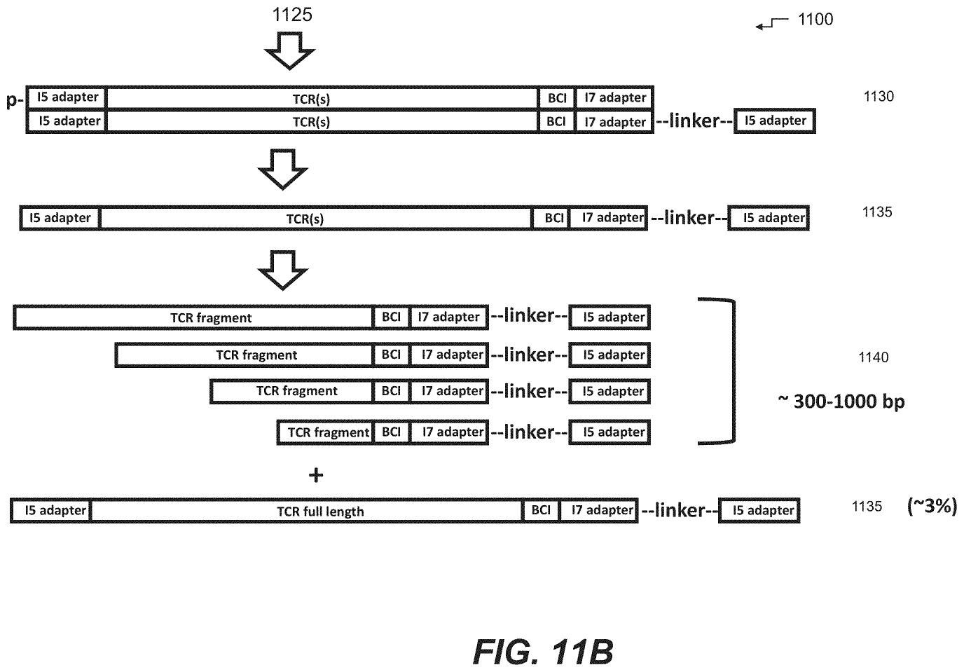

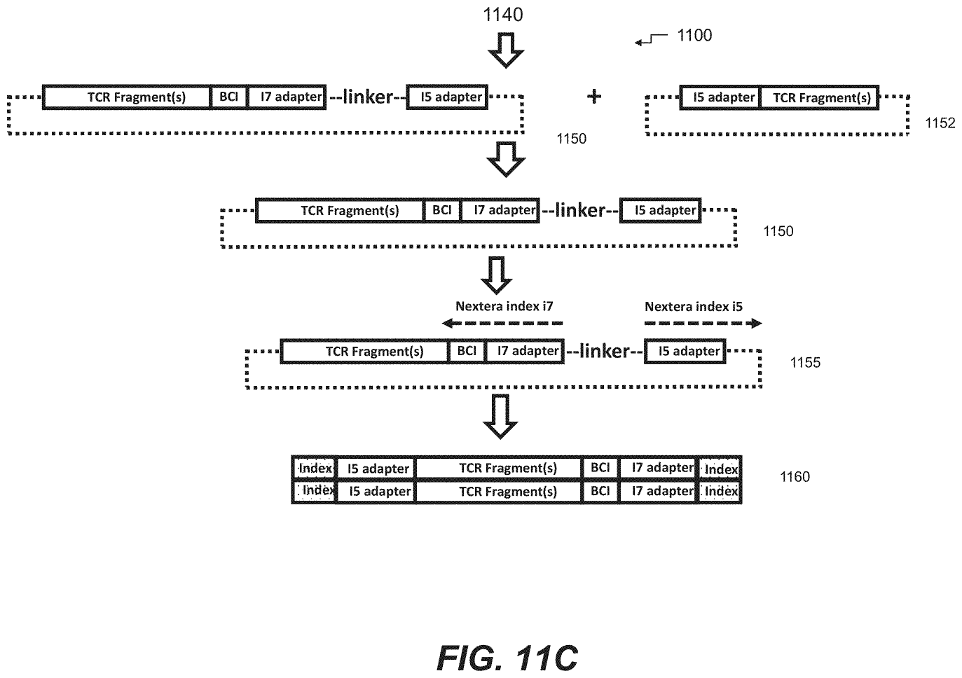

Referring to FIGS. 11A-11C, another method 1100 is provided for preparing a nucleic acid library for sequencing, including obtaining nucleic acid comprising mRNA molecules from a biological cell; and synthesizing cDNA 1110 from one or more of the mRNA molecules, which may be performed in any suitable manner as described above. The cDNA molecules 1110 are amplified (reaction complex 1115) providing amplified DNA molecules 1120. In some embodiments, a nested TSO primer 1106 may be used in the amplification. In various embodiments, gene specific primers 1108 may be used in the amplification.

Each of the amplified DNA molecules 1120 comprises a first portion having a 5′ terminus, a second portion, and a third portion having a 3′ terminus (forward strand). Each of the amplified DNA molecules 1120 includes a first priming sequence proximal to the 5′ terminus in the first portion of the molecule. The first priming sequence may start at the 5′ terminus, or there may be one or more nucleotides 5′ to the start of the promoter sequence. In various embodiments, the first portion may have a phosphate moiety at the 5′ terminus to increase the efficiency of the amplification. The third portion of the amplified DNA molecules 1120 includes a 3′ terminus and a second priming sequence proximal to the 3′ terminus. The second priming sequence of the third portion may be disposed such that the last nucleotide of the priming sequence is disposed at the 3′ terminus of the amplified DNA molecules or there may be one or more nucleotides disposed 3′ to the last nucleotide of the priming sequence of the third portion. Each of the amplified DNA molecules may further include a barcode (BCI) sequence within the third portion of the amplified DNA molecule. The barcode sequence may be located between the 3′ end of the second portion of the amplified DNA molecule and the 5′ end of the third portion of the amplified DNA molecule. The barcode sequence may be unique for mRNA isolated from the biological cell. In some embodiments, the barcode sequence may be unique for each RNA molecule captured from the biological cell. In some embodiments, the barcode may be any suitable barcode sequence. In some embodiments, the barcode may have a sequence of any one of SEQ ID NOS. 1-96.

The amplified DNA molecules 1120 includes the second portion, which includes a sequence of interest corresponding to a cDNA sequence (or portion thereof), wherein the second portion is disposed between the 3′ end of the first portion and the 5′ end of the third portion. In some embodiments, the second portion comprises a 5′ region having an unknown nucleic acid sequence and a 3′ region having a known nucleic acid sequence. In some embodiments, the gene specific primer primes the cDNA at a location within a known gene specific sequence. In some embodiment, the gene specific primer primes the cDNA at a location within a known 3′ constant region of the gene, thus providing a 3′ anchoring point for amplification. In some embodiments, the 3′ anchoring point for amplification may not be at a 3′ terminus of a known cDNA sequence of the cDNA. The 3′ region of the second portion of the amplified DNA molecules may be selected to be shorter than a complete known DNA sequence for the mRNA. For example, only a small or selected portion of the known region (e.g., a sequence encoding a constant domain of a heavy chain or light chain of an antibody) may be included within the chosen anchoring point, providing smaller amplicons for sequencing. This may provide fewer errors in sequencing. In some embodiments, only about 10%, 15%, 20%, 25%, 30%, 35%, 40%, 45%, 50%, 55%, 60%, 65%, 70% or about 75% of the known region of the gene may be included by preselection of the 3′ anchoring point.

A second round of amplification is subsequently performed, as shown in reaction complex 1125, using forward primer 1106 and a specialized reverse primer 1112, which contains two priming sequences linked by a linker containing at least one non-nucleotide moiety, providing linker-modified amplified DNA molecules 1130. The “bottom” strand (i.e., the stand formed by the specialized reverse primer 1112) of the linker-modified amplified DNA molecules 1130 includes a first portion having a 5′ terminus including a third priming sequence at the 5′ terminus, which is disposed proximal to the 3′ end of the first portion and is linked via a linker containing at least one non-nucleotide moiety to a fourth priming sequence. The first portion may further include the complement to the barcode sequence, which is disposed at the 3′ end of the first portion. The bottom strain of the amplified DNA includes a third portion which contains a complement to the first priming sequence of first portion of the amplified DNA molecules 1120. The “bottom strand” also includes a second portion comprising a sequence of interest corresponding to a cDNA sequence, wherein the second portion is disposed between the 3′ end of the first portion and the 5′ end of the third portion, and wherein the second portion comprises a complement to the 5′ region having an unknown nucleic acid sequence and a complement to the 3′ region having a known nucleic acid sequence.

The linker-modified amplified DNA molecules 1130 are treated with an exonuclease to remove the phosphorylated “top” strand, to provide single stranded linker-modified DNA molecules 1135.

At least a portion of the single stranded linker-modified DNA molecules 1135 are fragmented to provide truncated DNA molecules 1140, wherein the DNA is truncated within the unknown or variable region of the DNA. Fragmenting is performed enzymatically, and may use any suitable nuclease. In some embodiments, a nuclease such as DNaseI (New England Biolabs) may be used. As these molecules are the bottom strand only, the sequences are truncated within the complement of the 5′ region of the cDNA sequence corresponding to the RNA molecule captured from the biological cell. Thus, the third portion of the bottom strand DNA molecule is removed.

Each of the truncated linker-modified “bottom strand” DNA molecules are circularized using a circligase to provide a plurality of circularized DNA molecules 1150, each comprising the truncated sequence of interest and the specialized reverse priming sequence, which includes the third priming sequence linked via the linker containing at least one non-nucleotide moiety to the fourth priming sequence. A portion of non-truncated bottom strand DNA molecules may be included in the circularizing reaction, and may be about 1%, 3%, 5%, 7% or about 9% of the total amount of DNA circularized. Barcodes are retained as in the linear bottom strand DNA molecules 1140. A side product 1152, formed from the excised portions of DNA molecules 1140 are produced, but will not provide useful product in further processing.

The plurality of circularized DNA molecules 1150 are amplified in reaction complex 1155 to produce double stranded 5′ truncated DNA library molecules 1160. In certain embodiments, the fourth priming sequence comprises a binding site for a reverse primer sequence and the third priming sequence constitutes a forward primer sequence, thereby providing a plurality of 5′ truncated DNA library molecules. Each 5′ truncated DNA library molecule 1160 comprises (with reference to the bottom strand in FIG. 11c) a first portion comprising the third priming sequence, wherein the third priming sequence is proximal to a 5′ terminus, a third portion comprising the fourth priming sequence, wherein the fourth priming sequence is proximal to a 3′ terminus, and a second portion comprising a 5′ truncated sequence of interest. The third and the fourth priming sequences may include adapter sequences configured for parallel sequencing.

In some embodiments, the 5′ truncated DNA molecules may range in length, randomly less than a full length of the 5′ region having the unknown nucleic acid sequence. Each 5′ truncated DNA library molecule of the plurality may include the same 3′ region having the known nucleic acid sequence.

The plurality of 5′ truncated DNA library molecules may be a gene specific 5′ truncated DNA library. The plurality of 5′ truncated DNA library molecules may include a 5′ truncated DNA library encoding a TCR or BCR sequence. The TCR or BCR 5′ truncated DNA library may include both heavy and light chain sequences.

This experiment may be adapted for 5′ anchored sequencing by changing the site at which the specialized reverse primer is introduced (i.e., introducing the specialized reverse priming sequence to the top strand rather than the bottom strand), and enzymatically fragmenting the correspondingly adapted amplicons. The remainder of the process may be performed as described.

The libraries produced by this method may be sequenced by any suitable method. The read sequences may be tiled to reconstruct the full-length sequence of the RNA molecule captured from the biological cell. The RNA molecule may be any kind of RNA molecule, and some non-limiting examples include a TCR or BCR oligonucleotide sequence. The TCR or BCR oligonucleotide sequence may be a heavy chain or a light chain oligonucleotide sequence. In various embodiments, the read sequences are about 150 bp in length.

Oligonucleotide Sequences and Optional Capture Objects Comprising Same for Use in the Methods.

Many different types of oligonucleotides are useful in the methods described herein, and include classes of oligonucleotides as follows.

Barcode Sequence.

A barcode sequence is “non-identical” to other barcode sequences in a set when the n (e.g., three or more) oligonucleotide sequence of any one barcode sequence in the set of barcode sequences do not completely overlap with the n oligonucleotide sequence of any other barcode sequence in the set of barcode sequences; partial overlap (e.g., up to n−1, n−2, or n−3, as desired) is permissible, so long as each barcode sequence in the set is different from every other barcode sequence in the set by a minimum of 1, 2, 3, 4 or more oligonucleotides In certain embodiments, all (or substantially all) of the oligonucleotide sequences in the defined set will have the same length (or number of nucleotides). For example, the oligonucleotides sequences in the defined set can all have a length of 6 nucleotides. However, other lengths are also suitable for use in the present invention, ranging from about 4 nucleotides to about 15 nucleotides. Thus, for example, each oligonucleotide sequence in the defined set, for substantially all oligonucleotide sequences in the defined set, can have a length of 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. Alternatively, each or substantially all oligonucleotide sequences in the defined set may have length of 4-6, 6-8, 7-9, 8-10, 9-11, 10-12, 11-12, 12-14, or 13-16 nucleotides.

Each oligonucleotide sequence selected from the defined set of oligonucleotide sequences (and, thus, in a barcode sequence) can be said to be “non-identical” to the other oligonucleotide sequences in the defined set (and thus, the barcode sequence) because each oligonucleotide sequence can be specifically identified as being present in a barcode sequence based on its unique nucleotide sequence, which can be detected by (i) sequencing the barcode sequence, and/or (ii) performing a hybridization reaction with a probe (e.g., hybridization probe) that contains an oligonucleotide sequence that is complementary to the oligonucleotide sequence of the barcode, as described in further detail in WO2018/064640 A1, (Soumillon et al.), herein incorporated by reference in its entirety.

Each of the oligonucleotide sequences may be selected from a set of at least 12 oligonucleotide sequences. For example, the set can include at least 12, 15, 16, 18, 20, 21, 24, 25, 27, 28, 30, 32, 33, 35, 36, 39, 40, 42, 44, 45, 48, 50, 51, 52, 54, 55, 56, 57, 60, 63, 64, 65, 66, 68, 69, 70, 72, 75, 76, 78, 80, 81, 84, 85, 87, 88, 90, 92, 93, 95, 96, 99, 100, or more, including any number in between any of the foregoing. In some embodiments, the set of barcode sequences may consist essentially of 64, 81, 100, 125, 216, 256, 343, 512, 625, 729, 1000, 1296, 2401, 4096, 6561, or 10,000 barcode sequences.

A set of ninety six oligonucleotide sequences SEQ ID. Nos. 1-96 as shown in Table 1 has been designed for use in the methods The set was designed using the barcode generator python script from the Comai lab: (http://comailab.genomecenter.ucdavis.edu/index.php/Barcode generator) However, the methods described herein are not limited to use of the barcodes listed below, but may use any suitable set of barcodes as one of skill can devise.

TABLE 1
BARCODES.
This listing includes exemplary barcodes
suitable for use in the methods.

| SEQ | | SEQ | | SEQ | |
|---|---|---|---|---|---|
| ID | | ID | | ID | |
| NO. | SEQUENCE | NO. | SEQUENCE | NO. | SEQUENCE |
| 1 | AAAACT | 33 | TAGTAA | 65 | CCCCGT |
| 2 | AGATTA | 34 | TATAGA | 66 | CCCTGG |
| 3 | ATAAAC | 35 | TATGAA | 67 | CCGGAC |
| 4 | ATACAA | 36 | TATTGT | 68 | CCGTCG |
| 5 | AAAGTT | 37 | TGTTTA | 69 | CCTGGC |
| 6 | AAATTG | 38 | CACCAA | 70 | CGAGGC |
| 7 | AAGATT | 39 | CCACAT | 71 | CGCCCT |
| 8 | AATACA | 40 | CTAGTG | 72 | CGCGCA |
| 9 | AATCTT | 41 | TTAATC | 73 | CGGTGG |
| 10 | AATTCT | 42 | TTAGTT | 74 | GCGAGC |
| 11 | ACAATA | 43 | TTATTG | 75 | GCGCTG |
| 12 | ACTTAT | 44 | TTGAAA | 76 | GCGGTC |
| 13 | ATATAG | 45 | TTTACA | 77 | CGTGGG |
| 14 | CTTTAA | 46 | TTTCTT | 78 | CTGCGG |
| 15 | GATAAT | 47 | TTTTGA | 79 | GACCGC |
| 16 | GTAATA | 48 | AGACCT | 80 | GAGCGG |
| 17 | ATCAAA | 49 | GCTAGA | 81 | GCACGG |
| 18 | ATGAAT | 50 | AGGGGC | 82 | GCCAGG |
| 19 | ATTACT | 51 | CACGGC | 83 | GCCCTC |
| 20 | ATTCTA | 52 | CAGGGG | 84 | GCCGTG |
| 21 | ATTTCA | 53 | GGGATT | 85 | GCTCGC |
| 22 | CAAATA | 54 | GTTCGA | 86 | TCCCGC |
| 23 | CATTAT | 55 | TCTGCA | 87 | TCGGGC |
| 24 | CTATAT | 56 | CCAACC | 88 | TGGCCG |
| 25 | GTTTAT | 57 | GTACCG | 89 | GGAGCC |
| 26 | TCATAT | 58 | ACCGGC | 90 | GGCCAC |
| 27 | TCTTAA | 59 | ACGGGG | 91 | GGCGTC |
| 28 | TGATTT | 60 | AGCGGG | 92 | GGGCAG |
| 29 | TAAAGT | 61 | CCCACG | 93 | GGGGAC |
| 30 | TAAGAT | 62 | CGCTGC | 94 | GGGTCG |
| 31 | TAATGA | 63 | CGGCCA | 95 | GGTGGC |
| 32 | TACTAT | 64 | CGGGCT | 96 | GTGCGC |

Capture Oligonucleotide.

A capture oligonucleotide is an oligonucleotide sequence having from about 6 to about 50 nucleotides. In some embodiments, the capture oligonucleotide sequence captures a nucleic acid by hybridizing to a nucleic acid released from a cell of interest. One non-limiting example includes polyT sequences, (having about 30 to about 40 nucleotides) which can capture and hybridize to RNA fragments having PolyA at their 3′ ends. In other embodiments, the polyT sequence may further contain two nucleotides VI at its 3′ end, which as is described more fully below, may assist in capturing more RNA relative to more commonly known polyT sequences having two nucleotides VN at a 3′ end. Other examples of capture oligonucleotides include random hexamers (“randomers”) which may be used in a mixture to hybridize to and thus capture complementary nucleic acids. Alternatively, complements to gene specific sequences may be used for targeted capture of nucleic acids, such as B cell receptor or T cell receptor sequences.

Primer.

A primer, as referred to herein, is a single stranded oligonucleotide, and may be DNA or RNA. A primer may typically be about 10 to about 30, about 12 to about 28, about 15 to about 25, about 18 to about 22, about 18 to about 20 nucleotides in length, or any number therebetween. Primers may be provided in primer pairs, to prime both strands (top, bottom) of a double stranded DNA, and provide a starting point for DNA replication (e.g., strand extension). A primer may be a universal primer, a degenerate primer, or a specific primer.

Priming Sequence.

A priming sequence as referred to herein may be DNA or RNA, depending on the context, and is the sequence of nucleotides to which the primer binds. The capture oligonucleotide may have a priming sequence, and the priming sequence may be adjacent to or comprises the 5′-most nucleotide of the capture oligonucleotide(s). The priming sequence may be a generic or a sequence-specific priming sequence. The priming sequence may bind to a primer that, upon binding, primes a reverse transcriptase or a polymerase

Additional Priming and/or Adapter Sequences.

The capture oligonucleotide(s) or primer(s) used in these methods may optionally have one or more additional priming/adapter sequences, which either provide a landing site for primer extension or a site for immobilization to complementary hybridizing anchor sites within a parallel sequencing (e.g., massively parallel sequencing, high throughput sequencing or next generation sequencing) array or flow cell. Any suitable priming sequences may be used, which may be devised to be compatible with any type of NGS library preparation/sequencing platform.

Template Switching Oligonucleotide.

A template switching oligonucleotide (TSO) as used herein, refers to an oligonucleotide that permits the terminal transferase activity of an appropriate reverse transcriptase, such as, but not limited to Moloney murine leukemia virus (MMLV), to use the deoxycytidine nucleotides added to anchor a template switching oligonucleotide. Upon base pairing between the template switching oligonucleotide and the appended deoxycytidines, the reverse transcriptase “switches” template strands from the captured RNA to the template switching oligonucleotide and continues replication to the 5′ end of the template switching oligonucleotide. Thus, a complete 5′ end of the transcribed RNA is included and additional priming sequences for further amplification may be introduced.

The TSO may further include biotin linked to its 5′ end of the oligonucleotide to increase efficiency.

Optional Oligonucleotide Sequences.

DNA molecules produced for use in the sequencing experiments described herein may also include additional indicia such as a pool Index sequence. The Index sequence is a sequence of 4 to 10 oligonucleotides which uniquely identify a set of nucleic acids as belonging to one experiment, permitting multiplex sequencing combining sequencing libraries from several different experiments to save on sequencing run cost and time, while still permitting deconvolution of the sequencing data, and correlation back to the correct experiment and source biological cells associated therein.

Capture Object.

In some embodiments, RNA may optionally be captured to capture oligonucleotides that are connected to a capture object. In some embodiments, where capture of RNA from a biological object may be performed within a microfluidic environment, capture to a capture object may be utilized. Further description of capturing nucleic acid from biological objects to capture objects, particularly RNA, may be found in WO2018/064640 (Soumillon et al.), filed on Sep. 29, 2017, and WO2018/076024 (Park et al.), filed on Oct. 23, 2017, each of which disclosures are herein incorporated by reference in its entirety. Each of these two patent application publications describe the use of capture objects, which may optionally be barcoded capture objects, within a microfluidic environment to capture objects. The description within these two patent application publications further includes detecting the barcode in-situ within the microfluidic device.

A capture object may include a plurality of capture oligonucleotides, wherein each of said plurality includes: a priming sequence which is a primer binding sequence; a capture sequence; and, optionally, a barcode sequence comprising an oligonucleotide sequence. In various embodiments, the capture object may include a plurality of capture oligonucleotides. Each capture oligonucleotide comprises a 5′-most nucleotide and a 3′-most nucleotide. In various embodiments, the priming sequence may be adjacent to or comprises said 5′-most nucleotide. In various embodiments, the capture sequence may be adjacent to or comprises said 3′-most nucleotide. Typically, the barcode sequence, if present, may be located 3′ to the priming sequence and 5′ to the capture sequence.

Capture Object Composition.

Typically, the optional capture object has a composition such that it is amenable to movement using a dielectrophoretic (DEP) force, such as a negative DEP force. For example, the capture object can be a bead (or similar object) having a core that includes a paramagnetic material, a polymeric material and/or glass. In some embodiments, the capture object may include a magnetic component (e.g., a magnetic bead). Alternatively, the capture object can be non-magnetic. The polymeric material may be polystyrene or any other plastic material which may be functionalized to link the capture oligonucleotide. The core material of the capture object may be coated to provide a suitable material to attach linkers to the capture oligonucleotide, which may include functionalized polymers, although other arrangements are possible. The linkers used to link the capture oligonucleotides to the capture object may be any suitable linker as is known in the art. The linker may include hydrocarbon chains, which may be unsubstituted or substituted, or interrupted or non-interrupted with functional groups such as amide, ether or keto-groups, which may provide desirable physicochemical properties. The linker may have sufficient length to permit access by processing enzymes to priming sites near the end of the capture oligonucleotide linked to the linker. The capture oligonucleotides may be linked to the linker covalently or non-covalently, as is known in the art. A nonlimiting example of a non-covalent linkage to the linker may be via a biotin/streptavidin pair.

The capture object may be of any suitable size, as long as it is small enough to passage through the flow channel(s) of the flow region and into/out of a sequestration pen of any microfluidic device as described herein. Further, the capture object may be selected to have a sufficiently large number of capture oligonucleotides linked thereto, such that nucleic acid may be captured in sufficient quantity to generate a nucleic acid library useful for sequencing. In some embodiments, the capture object may be a spherical or partially spherical bead and have a diameter greater than about 5 microns and less than about 40 microns. In some embodiments, the spherical or partially spherical bead may have a diameter of about 5, about 7, about 8, about 10, about 12, about 14, about 16, about 18, about 20, about 22, about 24, or about 26 microns.

When barcode sequences are included on a capture oligonucleotide on a capture object, each capture oligonucleotide attached to a capture object has the same barcode sequence, and in many embodiments, each capture object has a unique barcode sequence. Using capture beads having unique barcodes on each capture bead permits unique identification of the sequestration pen into which the capture object is placed. In experiments where a plurality of cells is placed within sequestration pens, often singly, a plurality of capture objects are also delivered and placed into the occupied sequestration pens, one capture bead per sequestration pen. Each of the plurality of capture beads has a unique barcode, and the barcode is non-identical to any other barcode of any other capture present within the microfluidic device. As a result, the cell (or, in some embodiments, cells) within the sequestration pen, will have a unique barcode identifier incorporated within its sequencing library.

Sequencing Libraries from One or More Cells within a Microfluidic Environment.

A library of nucleic acids as described herein may be made from nucleic acids of a biological cell which may be imported into a well plate for lysis and library preparation. The biological cell may be imported from a second wellplate or any kind of cell holder or imported from a microfluidic device. In some embodiments, the biological cell may be exported singly into the wellplate for lysis and library preparation. In other embodiments, the biological cell may be lysed within a microfluidic device; its nucleic acids captured to a capture object including at least one capture oligonucleotide within the microfluidic device and the nucleic acid-laden capture object may be exported singly or with other capture objects including nucleic acid from the microfluidic device to a wellplate for further processing. The microfluidic device may be any suitable microfluidic device and may further include any microfluidic device as described herein. Further description of nucleic acid isolation within a microfluidic device may be found in WO2018/064640 (Soumillon et al.), filed on Sep. 29, 2017, and WO2018/076024 (Park et al.), filed on Oct. 23, 2017, each disclosure of which is herein incorporated by reference in its entirety.

In various embodiments, the enclosure of the microfluidic device may further include a dielectrophoretic (DEP) configuration. Disposing the biological cell and/or disposing the capture object may be performed by applying a dielectrophoretic (DEP) force on or proximal to the biological cell and/or the capture object. Exporting the biological cell and/or exporting the nucleic acid-laden capture object may be performed by applying a dielectrophoretic (DEP) force on or proximal to the biological cell and/or the capture object.

Cells for Sequencing.

In some embodiments, the biological cell may be an immune cell, for example a T cell, B cell, NK cell, macrophage, and the like. In some embodiments, the biological cell may be a cancer cell, such as a melanoma cancer cell, breast cancer cell, neurological cancer cell, etc. In other embodiments, the biological cell may be a stem cell (e.g., embryonic stem cell, induced pluripotent (iPS) stem cell, etc.) or a progenitor cell. In yet other embodiments, the biological cell may be an embryo (e.g., a zygote, a 2 to 200 cell embryo, a blastula, etc.). None of these exemplary biological cells are limiting, rather, any suitable biological cell may be utilized in the methods.

In various embodiments, the biological cell may be a single biological cell. Alternatively, the biological cell can be a plurality of biological cells, such as a clonal population.

Sequencing Libraries from B Cells.

A B cell lymphocyte can be, for example, a CD27⁺ B cell or a CD138⁺ B cell. In some embodiments, the B cell is a memory B cell. In other embodiments, the B cell is a plasma cell. The B cell lymphocyte can be obtained from a mammal, such as a human, a rodent (e.g., a mouse, rat, guinea pig, gerbil, hamster), a rabbit, a ferret, livestock (e.g., goats, sheep, pigs, horses, cows), a llama, a camel, a monkey, or obtained from avian species, such as chickens and turkey. In some embodiments, the mammal has been immunized against an antigen of interest. In some embodiments, the animal has been exposed to or infected with a pathogen associated with the antigen of interest. In some embodiments, the animal has a cancer that is associated with an antigen of interest. In other embodiments, the animal has an auto-immune disease that is associated with the antigen of interest. The sample containing the B cell lymphocyte can be a peripheral blood sample (e.g., PBMCs), a spleen biopsy, a bone marrow biopsy, a lymph node biopsy, a tumor biopsy, or any combination thereof.

The sample containing the B cell lymphocyte can be treated (e.g., sorted, negatively and/or positively) to enrich for desired B cell lymphocytes. In some embodiments, the desired B cell lymphocytes are memory B cells. In other embodiments, the desired B cell lymphocytes are plasma cells. In some embodiments, the desired B cell lymphocytes express an IgG-type antibody. Thus, for example, the sample can be depleted of cell types other than B cell lymphocytes. Methods of depleting non-B cell cell types from samples are well known in the art, and include, for example, treating the sample with the DYNABEADS™ Untouched Human B Cells reagent (Thermo Fisher), the B Cell Isolation Kit (Miltenyi), the EasySep B Cell Enrichment Kit (EasySep), the RosetteSep Human B Cell Enrichment Cocktain (Stem Cell Technologies), or the like. Alternatively, or in addition, the sample containing the B cell lymphocyte can be sorted by fluorescence-associated cell sorting (FACS) to remove unwanted cell types and enriched for the desired cell types. The FACS sorting can be negative and/or positive. For example, the FACS sorting can deplete the sample of B cell lymphocytes expressing IgM antibodies, IgA antibodies, IgD antibodies, IgG antibodies, or any combination thereof. Alternatively, or in addition, the FACS sorting can enrich the sample for B cell lymphocytes that express CD27 (or some other memory B cell marker) or for B cell lymphocytes that express CD138 (or some other plasma cell marker). The sample containing the B cell lymphocyte can be provided in an enriched state (i.e., pre-treated) such that no treatment to enrich for desired B cell lymphocytes is required as part of the method. Alternatively, treating the sample containing the B cell lymphocyte to enrich for desired B cell lymphocytes can be performed as part of the methods of the invention.

The sample containing the B cell lymphocyte can be treated to reduce sticking of cells in the sample to a microfluidic device. For example, the sample can be treated with a DNase, such as Benzonase® Nuclease (Millipore). The DNase may contain minimal protease activity.

BCR Gene Sequences.

The B cell receptor gene sequence include several sub-regions including variable (V), diversity (D), joining (J) and constant (C) segments, in that order 5′ to 3′ in the released RNA. The constant region is just 5′ to the polyA sequence. In a number of approaches to sequencing BCR, it may be desirable to construct selection strategies to obtain amplicons for sequencing that do not contain the poly A sequence (tail). Further it may be desirable to produce amplicons which retain less than all of the oligonucleotide sequence of the constant region. Limiting amplification to exclude these sections of the released nucleic acid sequence can permit more robust sequencing of the V, D (if present), and J segments of the BCR. Additionally, use of the methods described herein, where the 5′ end of amplicons for BCR sequencing are differentially truncated and the 3′ end of the amplicons are anchored at a selected point within the constant region (and not at the 3′ terminus of the constant region) can provide more robust reconstruction of the V, D (if present), and J segments of the BCR gene sequence.

A sequencing library may be obtained from B cell lymphocytes of interest by methods other than the methods described herein. Other suitable, but non-limiting methods are described in WO2018/064640 (Soumillon et al.), filed on Sep. 29, 2017, and WO2018/076024 (Park, et al.), filed on Oct. 23, 2017, each of which disclosures are herein incorporated by reference in its entirety, and hereby incorporated by reference for all purposes in its entirety.

Sequencing Libraries from a T Lymphocyte (Cell).

The T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th1 and Th2 cells, CD8⁺ T cells (e.g., cytotoxic T cells), peripheral blood mononuclear cells (PBMCs), peripheral blood leukocytes (PBLs), tumor infiltrating cells (TILs), memory T cells, naive T cells, and the like. The T cell may be a CD8⁺ T cell or a CD4⁺ T cell.

In some embodiments, the predominant cell type in the population of T lymphocytes may be a naïve T cell (T_{naïve}), a memory T cell, such as a central memory T cell (T_{CM}) or an effector memory T cell (T_{EM}), or an effector T cell (T_{EFF}).

A sample that contains T lymphocytes may be pre-processed to enrich for naïve T lymphocytes, memory T lymphocytes (e.g., T_{CM} cells and/or T_{EM} cells), T_{EFF} lymphocytes, or any combination thereof. The processing can include removing debris and/or non-lymphocyte cell types from the sample. Alternatively, or in addition, the processing can include depleting the sample of naive T lymphocytes, memory T lymphocytes (such as T_{CM} and/or T_{EM} lymphocytes), T_{EFF} lymphocytes, or a combination thereof.

The sample can be from a subject, such as a subject that is suffering from cancer (e.g., any type of cancer described herein or known in the art). The sample can be a peripheral blood sample or a derivative thereof (e.g., a sample of PBMCs). Alternatively, the sample can be a solid tumor biopsy or FNA. In some embodiments, a peripheral blood sample is processed to enrich for naïve T lymphocytes. In other embodiments, a tumor sample is processed to enrich for memory T lymphocytes, particularly T_{CM} lymphocytes although T_{EF} lymphocytes may be enriched. In still other embodiments, a tumor sample is processed to enrich for T_{EFF} lymphocytes. To enrich for the desired T lymphocyte cell type(s), binding agents (e.g., antibodies or the like) that specifically bind to one or more cell surface antigens can be employed. The cell surface antigens bound by the binding agents can be any suitable cell surface antigen, including, but not limited to CD2, CD4, CD8, CD28, CD45RO, CD45RA, CCR7, CD62L, PD-1, and CD137T, or combinations thereof. The processing can comprise contacting the sample with one or more fluorescently labeled binding agents and performing FACS to select for labeled cells (e.g., if enriching based on the cell surface antigen(s) specifically bound by the one or more binding agents) or to remove labeled cells (e.g., if depleting based on the cell surface antigen(s) specifically bound by the one or more binding agents). Alternatively, or in addition, the processing can comprise contacting the sample with one or more binding agents that are linked to a solid support, and removing cells bound to the solid support (e.g., if depleting based on the cell surface antigen(s) specifically bound by the one or more binding agents) or removing cells that not bound to the solid support (e.g., if enriching based on the cell surface antigen(s) specifically bound by the one or more binding agents). The solid support can be, for example, one or more beads (e.g., a population of beads, which may be magnetic). If magnetic beads are used, a magnetic force can be applied to the sample such that the magnetic beads form a pellet, allowing a resulting supernatant to be separated from the pellet.

T Cell Receptor (TCR):

TCR genes encode, in many individuals, for an alpha chain and a beta chain where each of the alpha and the beta chains have a constant region and a variable region. The methods as described herein may permit a more robust coverage of the variable regions of each chain.

Kits.

Kits are provided herein for preparing a nucleic acid library. The kit may include a capture oligonucleotide for capturing an mRNA molecule; a gene specific primer; and a fragmenting reagent. The RNA capture oligonucleotide may be like any RNA capture oligonucleotide described herein. In some embodiments the RNA capture oligonucleotide may have a dTVI sequence at its 3′ terminus. The RNA capture oligonucleotide may include a priming sequence at or proximal to a 5′ terminus.

In various embodiments of the kit, the gene specific primer may be specific for a TCR or a BCR sequence. The TCR or BCR gene specific primer may prime both heavy and light chain sequences of the TCR or BCR gene.

In various embodiments of the kit, the fragmenting reagent is a chemical fragmentation reagent or an enzymatic fragmentation reagent. The chemical fragmentation reagent may be any suitable chemical fragmentation reagent as is known in the art, and may include a divalent cation. The divalent cation may be magnesium and/or zinc. When the fragmenting reagent is an enzymatic fragmentation reagent, the enzymatic fragmentation reagent may include a non-specific nuclease, a restriction endonuclease, or a tagmentation reagent comprising a transposase. Any suitable non-specific nuclease may be used for this process, and in some embodiments, the non-specific nuclease may be DNase 1.

In various embodiments of the kit, the kit may include a reverse transcriptase. In some other embodiments, the kit may include a circularizing enzyme, such as a ligase engineered to intramolecularly ligate ends of a linear oligonucleotide to prepare circular oligonucleotides.

In yet other embodiments, the kit may include sets of primers for use in the methods, which may be any primer described herein or may be any other suitable primer for any of the processes. When the method of preparing a nucleic acid library includes a process for fragmenting amplified DNA with subsequent circularization, the kit may include a primer having a first priming sequence proximal to a 5′ terminus of the primer linked via a non-nucleotide linker to a second priming sequence proximal to a 3′ terminus of the primer.

The primers may further include a barcode. The barcode may be any suitable barcode, or may be one of the barcodes shown in Table 1, having a sequence of any of SEQ ID NOS. 1-96. The kits may further include any other reagent described for use in one or more of the processes of the methods described herein.

Microfluidic Devices and Systems for Operating and Observing Such Devices.

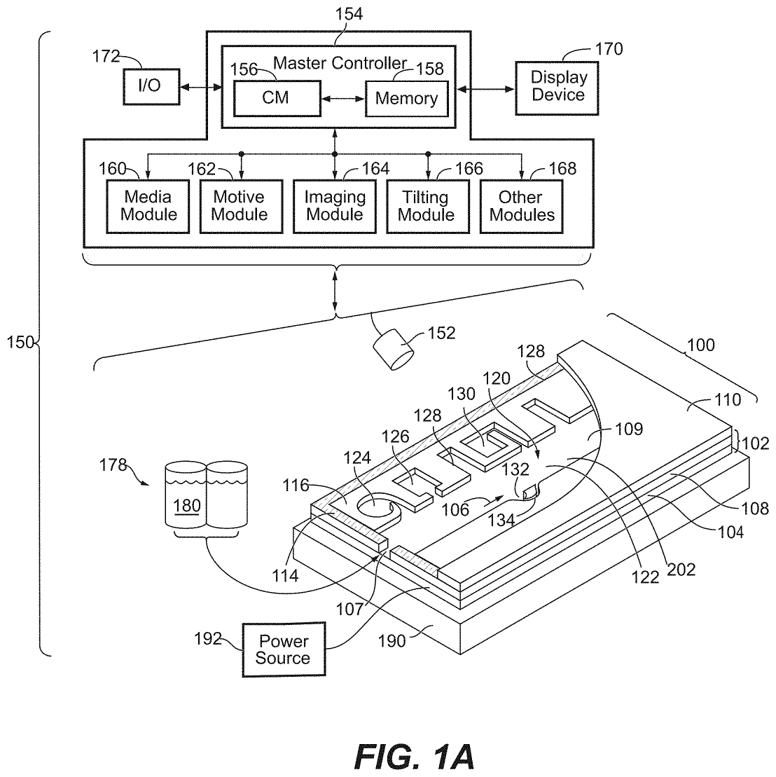

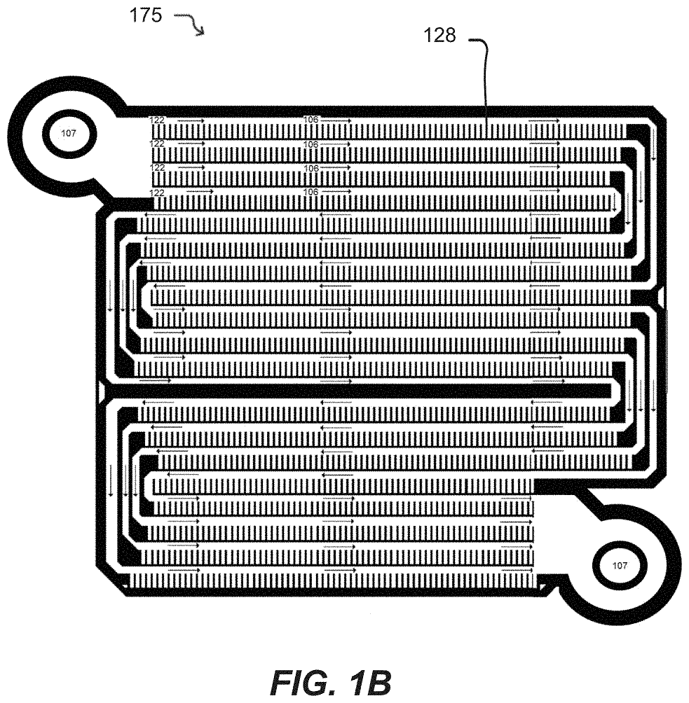

It should be appreciated that various features of microfluidic devices, systems, and motive technologies described herein may be combinable or interchangeable. For example, features described herein with reference to the microfluidic device 100, 175, 200, 300, 320, 400, 520 and system attributes as described in FIGS. 1A-5B may be combinable or interchangeable as deemed suitable for its purpose.

Microfluidic Devices.

FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A, but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include a sub-class including sequestration pens), traps, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material.

The microfluidic circuit material 116 can be patterned with cavities or the like to define the circuit elements and interconnections of the microfluidic circuit 120, such as chambers, sequestration pens and microfluidic channels. The microfluidic circuit material 116 can comprise a flexible material, such as a flexible polymer (e.g. rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g. photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and fluidically connected thereto. A chamber can have one or more openings fluidically connecting the chamber with one or more flow regions. In some embodiments, a flow region corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pen may have one or more openings. In some embodiments of sequestration pens, a sequestration pen may have only a single opening in fluidic communication with the flow path 106. In some embodiments, the sequestration pens comprise various features and structures that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single channel 122 and flow path 106 is shown. However, other embodiments may contain multiple channels 122, each configured to comprise a flow path 106. The microfluidic circuit 120 further comprises an inlet valve or port 107 in fluid communication with the flow path 106 and fluidic medium 180, whereby fluidic medium 180 can access channel 122 via the inlet port 107. In some instances, the flow path 106 comprises a single path. In some instances, the single path is arranged in a zigzag pattern whereby the flow path 106 travels across the microfluidic device 100 two or more times in alternating directions. The flow in the flow path may proceed from inlet to outlet or may be reversed and proceed from outlet to inlet.

One example of such a multi-channel device, microfluidic device 175, is shown in FIG. 1B, which may be like microfluidic device 100. The microfluidic device 175 and its constituent circuit elements (e.g. channels 122 and sequestration pens 128) may have any of the dimensions discussed herein. The microfluidic circuit illustrated in FIG. 1B has two inlet ports 107 and four distinct channels 122, each containing a distinct flow path 106. The microfluidic device 175 further comprises a plurality of sequestration pens opening off of each channel 122, where each of the sequestration pens may be similar to sequestration pen 128 of FIG. 1A, and may have any of the dimensions or functions of any sequestration pen as described herein. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object.

Sequestration Pens.

The microfluidic devices described herein may include sequestration pens, where each sequestration pen is suitable for holding one or more micro-objects (e.g., biological cells, oocytes or embryos). The sequestration pens may be disposed within and open to a flow region, which in some embodiments is a microfluidic channel. Each of the sequestration pens can have one opening for fluidic communication to a microfluidic channel.

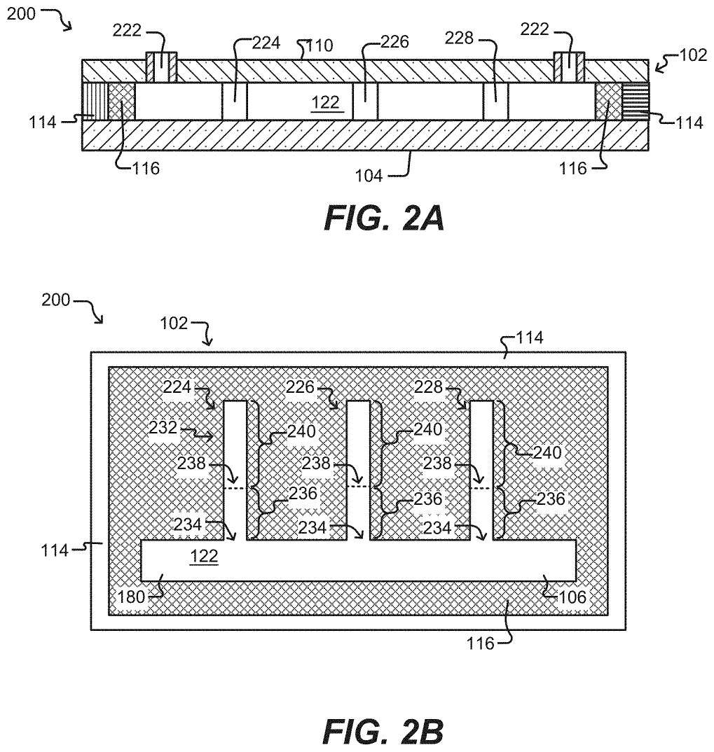

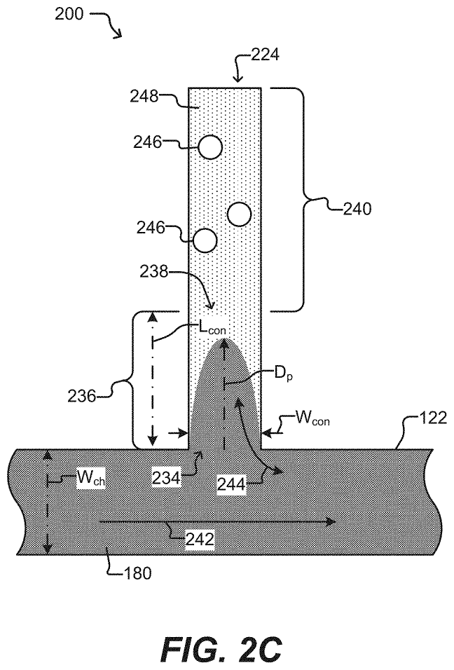

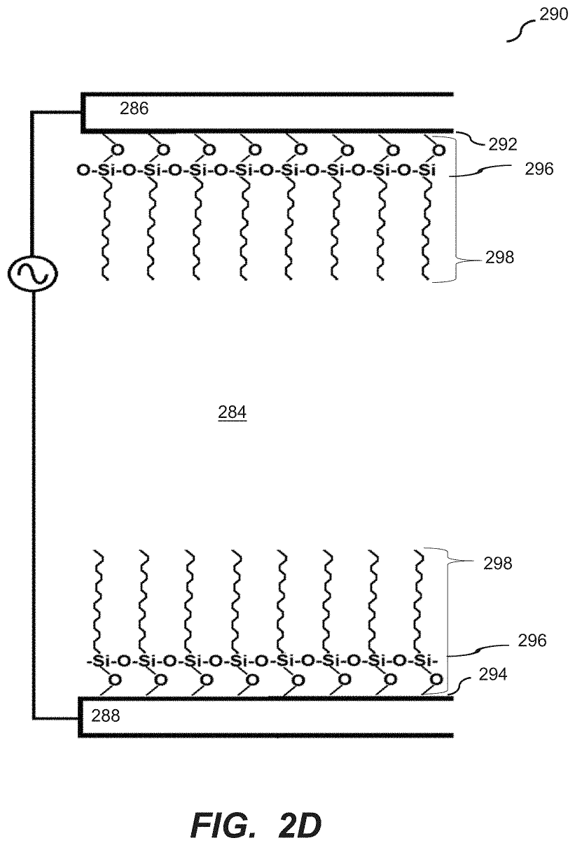

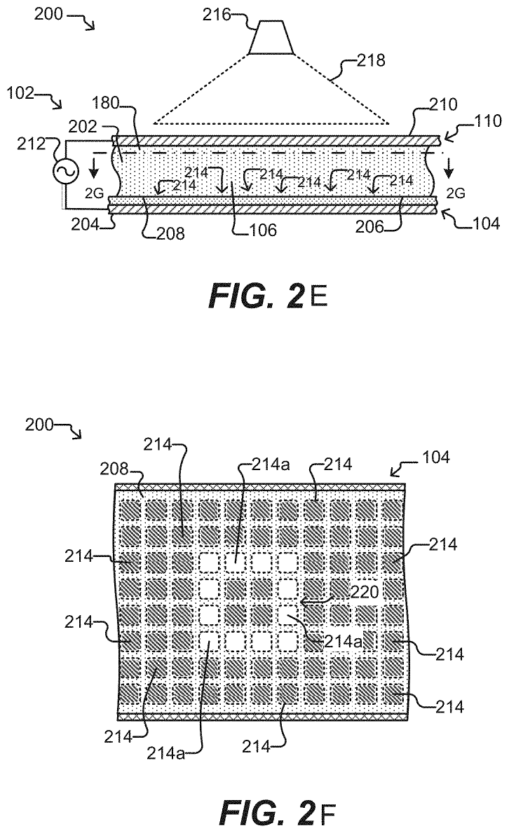

FIGS. 2A-2C show sequestration pens 224, 226, and 228 of a microfluidic device 200, which may be like sequestration pen 128 of FIG. 1A. Each sequestration pen 224, 226, and 228 can comprise an isolation structure 232 defining an isolation region 240 and a connection region 236 fluidically connecting the isolation region 240 to a flow region, which may, in some embodiments be a microfluidic channel, such as channel 122. The connection region 236 can comprise a proximal opening 234 to the flow region (e.g., microfluidic channel 122) and a distal opening 238 to the isolation region 240. The isolation region 240 has only one opening, which opens to the connection region 236, thereby fluidically connecting the isolation region 240 to the flow region. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, turbulence from the flow in the microfluidic channel does not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which is a side elevation of microfluidic device 200. FIG. 2B shows a top view of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both of the microfluidic channel 122 (or flow region) and sequestration pens may be less than about 3%, 2%, 1%. 0.9%, 0.8%, 0.5%, 0.3% or 0.1% of the height of the walls of the sequestration pen or walls of the microfluidic device. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 of fluidic medium 180 in the microfluidic channel 122 can be selectively generated and stopped. For example, as shown, the ports 222 can be disposed at different locations (e.g., opposite ends) of the microfluidic channel 122, and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of sequestration pen 224 can cause a secondary flow 244 of the fluidic medium 180 into and/or out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) may be greater than the penetration depth Dₚ of the secondary flow 244 into the connection region 236. The penetration depth Dₚ may depend on the velocity of the fluidic medium 180 in the channel 122, and in some embodiments, the viscosity of fluidic medium 180. The penetration depth Dₚ of the secondary flow 244 may additionally depend upon dimensions and/or orientation of the microfluidic channel 122 and the proximal opening 234 of the connection region 236 to the microfluidic channel 122. For example, Dₚ may depend upon the shape of the microfluidic channel 122, which may be defined by a width W_{ch} (or cross-sectional area) of the microfluidic channel 122 at the proximal opening 234; a width W_{con} (or cross-sectional area) of the connection region 236 at the proximal opening 234 (which can be the same as the width or cross-sectional area of the proximal opening 234); a height H_{ch} of the channel 122 at the proximal opening 234; and/or the width (or cross-sectional area) of the distal opening 238.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit. Thus the micro-objects may be retained within the isolation region 240. Selection of the operating parameters (e.g., velocity of fluidic medium 180) and along with selection of microfluidic circuit element dimensions may further prevent contamination of the isolation region 240 of sequestration pen 224 by materials from the microfluidic channel 122 or another sequestration pen 226 or 228.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g. a portion of the connection region adjacent to the proximal opening 234).

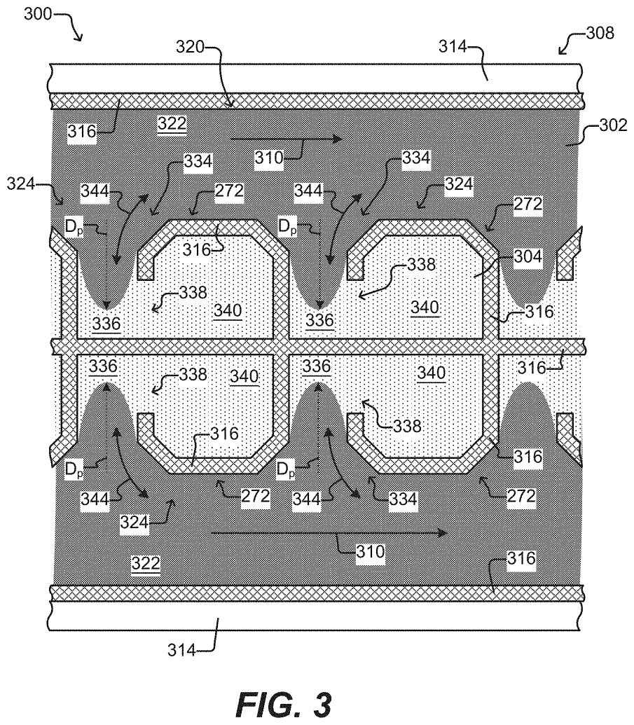

FIG. 3 depicts another exemplary embodiment of a microfluidic device 300 containing a microfluidic circuit 320 and microfluidic channel 322. The microfluidic device 300 comprises a support structure (not visible in FIG. 3) which may be the same or generally similar to the support structure 104 of device 100 depicted in FIG. 1A, a microfluidic circuit structure 308, and a cover (not visible in FIG. 3), which can be the same or generally similar to the cover 110 of device 100 depicted in FIG. 1A.

The microfluidic circuit structure 308 includes a frame 314 and microfluidic circuit material 316, which can be like frame 114 and microfluidic circuit material 116 of device 100. As shown in FIG. 3, the microfluidic circuit 320 defined by the microfluidic circuit material 316 can comprise multiple channels 322 (two are shown but there can be more) to which multiple sequestration pens 324, also formed from microfluidic circuit material 316, can be fluidically connected. Each sequestration pen 324 can comprise an isolation structure 272, an isolation region 340 within the isolation structure 272, and a connection region 336. The connection region 336 has a proximal opening 334 at the microfluidic channel 322 and a distal opening 338 to the isolation region 340 of the sequestration pen 324 fluidically connecting the isolation region 270 to the microfluidic channel 322. Isolation region may contain a second fluidic medium 304. Similarly to the sequestration pens 224, 226, 228, a flow 310 of a first fluidic medium 302 in a channel 322 can create a secondary flow 344 of the first medium 302 having a penetration depth Dₚ from the microfluidic channel 322 into and/or out of the respective connection regions 336 of the sequestration pens 324. The connection region 336 of each sequestration pen 324 can include the area extending between the proximal opening 334 to a channel 322 and the distal opening 338 to the isolation region 340.

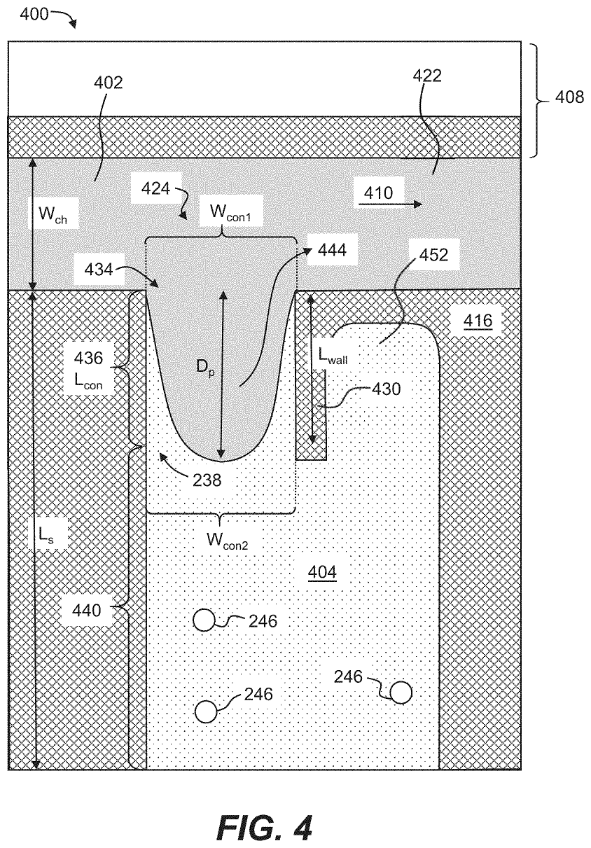

FIG. 4 depicts another exemplary embodiment of a microfluidic device 400, respectively, containing microfluidic circuit structure 408, which includes a channel 422 and sequestration pen 424, which has features and properties like any of the sequestration pens described herein for microfluidic devices 100, 200, 300, 320 and the like.

The exemplary microfluidic devices of FIG. 4 includes a microfluidic channel 422, having a width W_{ch}, as described herein and containing a flow 410 of first fluidic medium 402 and one or more sequestration pens 424 (only one illustrated in FIG. 4). The sequestration pens 424, each having a length Lₛ, a connection region 436, and an isolation region 440, where the isolation region 440 contains a second fluidic medium 404. The connection region 436 has a proximal opening 434, having a width W_{con1}, which opens to the microfluidic channel 422, and a distal opening 438, having a width W_{con2}, which opens to the isolation region 440. The width W_{con}1 may or may not be the same as W_{con2}, as described herein. The isolation structure of each sequestration pen 424 may be formed of microfluidic circuit material 416, which further include connection region walls 430. A connection region wall 430 can correspond to a structure that is laterally positioned with respect to the proximal opening 434 and at least partially extends into the enclosed portion of the sequestration pen 424. In some embodiments, the length L_{con} of the connection region 436 is at least partially defined by length L_{wall} of the connection region wall 430. The connection region wall 430 may have a length L_{wall}, selected to be more than the penetration depth Dₚ of the secondary flow 444. The secondary flow 444 can be wholly contained within the connection region without extending into the isolation region 440.

The connection region wall 430 may define a hook region 452, which is a sub-region of the isolation region 440 of the sequestration pen 424. Since the connection region wall 430 extends into the inner cavity of the sequestration pen, the connection region wall 430 can act as a physical barrier to shield hook region 452 from secondary flow 444, with selection of the length of L_{wall}, contributing to the extent of the hook region. In some embodiments, the longer the length L_{wall} of the connection region wall 430, the more sheltered the hook region 452.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Sequestration Pen Dimensions.

Various dimensions and/or features of the sequestration pens and the microfluidic channels to which the sequestration pens open, as described herein, which may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure, as follows:

According to some embodiments, the flow of fluidic medium within the microfluidic channel (e.g., 122 or 264) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, or 15 microliters/sec. The foregoing are examples only, and the flow of fluidic medium within the microfluidic channel can have a maximum velocity (e.g., Vₘₐₓ) selected to be a value between any of the values listed above.

The microfluidic channel of a microfluidic device to which a sequestration pen opens may have specified size (e.g., width or height). In some embodiments, the width (e.g., W_{ch}) of the microfluidic channel at the proximal opening of a sequestration pen can be about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen.

The height H_{ch} of the microfluidic channel at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{ch} of the microfluidic channel (e.g., 122) can be selected to be between any of the values listed above. The height H_{ch} of the microfluidic channel 122 can be selected to be any of these heights in regions of the microfluidic channel other than at a proximal opening of a sequestration pen.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The proximal opening of a sequestration pen may have a width (e.g., W_{con} or W_{con1}) that is at least as large as the largest dimension of a micro-object (e.g., biological cell which may be a T cell, B cell, or an ovum or embryo) that the sequestration pen is intended for. In some embodiments, the proximal opening has a width (e.g., W_{con} or W_{con1}) of about 30 microns, about 50 microns, about 60 microns, about 100 microns, about 200 microns, about 300 microns or may be about 50-300 microns, about 50-200 microns, about 50-100 microns, about 75-150 microns, about 75-100 microns, or about 200-300 microns. In some embodiments, the proximal opening has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel. The foregoing are examples only, and width (e.g., W_{con} or W_{con1}) of a proximal opening can be selected to be a value between any of the values listed above.

In some embodiments, the width W_{ch} of the microfluidic channel (i.e., taken transverse to the direction of the flow of the first medium) can be substantially perpendicular to a width W_{con1} of the proximal opening and/or the width W_{con2} of the distal opening 238.

In some embodiments, the width W_{con1} of a proximal opening of a connection region of a sequestration pen may be the same as a width W_{con2} the distal opening to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

A cross-sectional area of the microfluidic channel at a proximal opening of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, the cross-sectional area of the microfluidic channel at regions of the microfluidic channel other than at the proximal opening can also be selected to be between any of the values listed above. In some embodiments, the cross-sectional area is selected to be any one value between any of the values listed above for the entire length of the microfluidic channel.

In some embodiments, the proximal opening of a sequestration pen may have a width (e.g., W_{con} or W_{con1}) from about 20 microns to about 100 microns, and the connection region of the sequestration pen may have a length (e.g., L_{con}) from the proximal opening to the distal opening to the isolation region of the sequestration pen that is at least 1.0 times, at least 1.1 times, at least 1.2 times, at least 1.3 times, at least 1.4 times, at least 1.5 times, at least 1.7 times, at least 1.9 times, at least 2.0 times, at least 2.5 times, or at least 2.7 times the width (e.g., W_{con} or W_{con1}) of the proximal opening.

In some embodiments, a height (e.g., H_{ch}) of the microfluidic channel is from about 30 to about 50 microns, the proximal opening (e.g., 234 or 274) into the microfluidic flow region of the connection region (e.g., 236 or 268) of a sequestration pen may have a width W_{con1} from about 20 microns to about 60 microns, and the connection region (e.g., 236 or 268) may have a length (e.g., L_{con}) from the proximal opening (e.g., 234 or 274) to the distal opening (e.g., 238) to the isolation region (e.g., 240 or 270) of the sequestration pen that is as least 0.4 times, at least 0.5 times, or at least 1.0 times the width (e.g., W_{con} or W_{con1}) of the proximal opening (e.g., 234 or 274). In some embodiments, a ratio of the length (e.g., L_{con}) of a connection region (e.g., 236 or 268) to a width (e.g., W_{con} or W_{con1}) of the proximal opening (e.g., 234 or 274) can be greater than or equal to any of the following ratios: 0.5, 1.0, 1.5, 2.0, 2.5, 3.0, 3.5, 4.0, 4.5, 5.0, 6.0, 7.0, 8.0, 9.0, 10.0, or more. The foregoing are examples only, and the width (e.g., W_{ch}) of the microfluidic channel (e.g., 122 or 264), the length (e.g., L_{con}), and/or the width (e.g., W_{con} or W_{con1}) of the proximal opening (e.g., 234 or 274) can be a value selected to be between any of the values listed above.

According to some embodiments, a sequestration pen may have a specified height (e.g., Hₛ). In some embodiments, a sequestration pen Hₛ has a height of about 30 to about 200 microns, or about 50 to about 150 microns. The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. The height H_{con} of the connection region can be selected to be the same as the height H_{ch} of the microfluidic channel at the proximal opening of the connection region. Additionally, the height Hₛ of the sequestration pen can be selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above.

According to some embodiments, a sequestration pen of a microfluidic device may have a specified volume. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

The connection region wall of a sequestration pen may have a length (e.g., L_{wall}) that is at least 1.0 times, at least 1.1 times, at least 1.2 times, at least 1.3 times, at least 1.4 times, at least 1.5 times, at least 1.7 times, at least 1.9 times, at least 2.0 times, at least 2.5 times, at least 2.7 times, at least 2.9 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

In various embodiment, the microfluidic device has sequestration pens configured as in any of the embodiments discussed herein where the microfluidic device has about 5 to about 10 sequestration pens, about 10 to about 50 sequestration pens, about 100 to about 500 sequestration pens; about 200 to about 1000 sequestration pens, about 500 to about 1500 sequestration pens, about 1000 to about 2000 sequestration pens, about 1000 to about 3500 sequestration pens, about 3000 to about 7000 sequestration pens, about 5000 to about 10,000 sequestration pens, about 9,000 to about 15,000 sequestration pens, or about 12,000 to about 20,000 sequestration pens. The sequestration pens need not all be the same size and may include a variety of configurations (e.g., different widths, different features within the sequestration pen).

Coating Solutions and Coating Agents.

In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic Polymer-Based Coating Materials.

The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently Linked Coating Materials.

In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device having an EW or OEW mechanism included within the base, may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned Surface Properties.

Various factors can alter the physical thickness of the conditioned surface, such as the manner in which the conditioned surface is formed on the substrate (e.g. vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Without intending to be limited by theory, by presenting cationic moieties, anionic moieties, and/or zwitterionic moieties at the inner surfaces of the enclosure of the microfluidic circuit, the coating material can form strong hydrogen bonds with water molecules such that the resulting water of hydration acts as a layer (or “shield”) that separates the biological micro-objects from interactions with non-biological molecules (e.g., the silicon and/or silicon oxide of the substrate). In addition, in embodiments in which the coating material is used in conjunction with coating agents, the anions, cations, and/or zwitterions of the coating material can form ionic bonds with the charged portions of non-covalent coating agents (e.g. proteins in solution) that are present in a medium (e.g. a coating solution) in the enclosure.

Unitary or Multi-Part Conditioned Surface.

The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

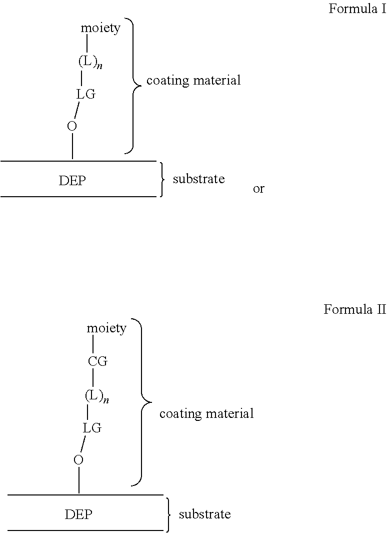

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

FIG. 2D depicts a cross-sectional view of a microfluidic device 290 having an exemplary covalently linked coating material providing a conditioned surface. As illustrated, the coating materials 298 (shown schematically) can comprise a monolayer of densely-packed molecules covalently bound to both the inner surface 292 of a base 286, which may be a DEP substrate, and the inner surface 294 of a cover 288 of the microfluidic device 290. The coating material 298 can be disposed on substantially all inner surfaces 294 and/or 292 proximal to, and facing inwards towards, the enclosure 284 of the microfluidic device 290, including, in some embodiments and as discussed above, the surfaces of microfluidic circuit material (not shown) used to define circuit elements and/or structures within the microfluidic device 290.

In the embodiment shown in FIG. 2D, the coating material 298 can include a monolayer of organosiloxane molecules, each molecule covalently bonded to the inner surfaces 292 and/or 294 of the microfluidic device 290 via a siloxy linker 296. Any of the above-discussed coating material 298 can be used, including but not limited to an alkyl-terminated, a fluoroalkyl terminated moiety, a PEG-terminated moiety, a dextran terminated moiety, or a terminal moiety containing positive and/or negative charges, where the terminal moiety is disposed at its enclosure-facing terminus (i.e. the portion of the monolayer of the coating material 298 that is not bound to the inner surfaces 292 and/or 294 and is proximal to the enclosure 284).

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic Device Motive Technologies.

The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise a DEP and/or an EW configuration for selectively inducing motive forces on micro-objects in the fluidic medium 180 in the microfluidic circuit 120 and thereby select, capture, and/or move individual micro-objects or groups of micro-objects. In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configurations can include those illustrated in U.S. Pat. No. RE 44,711 (Wu, et al., originally issued as U.S. Pat. No. 7,612,355), U.S. Pat. No. 7,956,339 (Ohta et al.), and U.S. Pat. No. 9,403,172 (Short et al.), each of which is incorporated herein by reference in its entirety. Examples of suitable OEW configurations can include those illustrated in U.S. Pat. No. 6,958,132 (Chiou et al.), and U.S. Pat. No. 9,533,306 (Chiou et al.), each of which is incorporated herein by reference in its entirety. Examples of suitable optically-actuated electrokinetic devices that include combined OET/OEW configurations can include those illustrated in U.S. Patent Application Publication No. 2015/0306598 (Khandros et al.), U.S. Patent Application Publication No 2015/0306599 (Khandros et al.), and U.S. Patent Application Publication No. 2017/0173580 (Lowe, et al.), each of which is incorporated herein by reference in its entirety.

It should be understood that for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 2E and 2F show a side cross-sectional view and a top cross-sectional view, respectively, of a portion of an enclosure 102 of the microfluidic device 200 having a region/chamber 202, which may be part of a fluidic circuit element having a more detailed structure, such as a growth chamber, a sequestration pen (which may be like any sequestration pen described herein), a flow region, or a flow channel. Furthermore, the microfluidic device 200 may include other fluidic circuit elements and may be part of a system including control and monitoring equipment 152, described above, having one or more of the media module 160, motive module 162, imaging module 164, optional tilting module 166, and other modules 168. Microfluidic devices 300, 400, may similarly have any of the features described in detail for FIGS. 1A-1B and 2E-2F.

As shown in the example of FIG. 2E, the microfluidic device 200 includes a support structure 104 having a bottom electrode 204 and an electrode activation substrate 206 overlying the bottom electrode 204, and a cover 110 having a top electrode 210, with the top electrode 210 spaced apart from the bottom electrode 204. The top electrode 210 and the electrode activation substrate 206 define opposing surfaces of the region/chamber 202. A fluidic medium 180 contained in the region/chamber 202 thus provides a resistive connection between the top electrode 210 and the electrode activation substrate 206. A power source 212 configured to be connected to the bottom electrode 204 and the top electrode 210 and create a biasing voltage between the electrodes, as required for the generation of DEP forces in the region/chamber 202, is also shown. The power source 212 can be, for example, an alternating current (AC) power source.

In certain embodiments, the microfluidic device 200 illustrated in FIGS. 2E and 2F can have an optically-actuated DEP configuration. Accordingly, changing patterns of light 218 from the light source 216, which may be controlled by the motive module 162, can selectively activate and deactivate changing patterns of DEP electrodes at regions 214 of the inner surface 208 of the electrode activation substrate 206. (Hereinafter the regions 214 of a microfluidic device having a DEP configuration are referred to as “DEP electrode regions.”) As illustrated in FIG. 2F, a light pattern 218 directed onto the inner surface 208 of the electrode activation substrate 206 can illuminate select DEP electrode regions 214a (shown in white) in a pattern, such as a square. The non-illuminated DEP electrode regions 214 (cross-hatched) are hereinafter referred to as “dark” DEP electrode regions 214. The relative electrical impedance through the DEP electrode activation substrate 206 (i.e., from the bottom electrode 204 up to the inner surface 208 of the electrode activation substrate 206 which interfaces with the fluidic medium 180 in the flow region 106) is greater than the relative electrical impedance through the fluidic medium 180 in the region/chamber 202 (i.e., from the inner surface 208 of the electrode activation substrate 206 to the top electrode 210 of the cover 110) at each dark DEP electrode region 214. An illuminated DEP electrode region 214a, however, exhibits a reduced relative impedance through the electrode activation substrate 206 that is less than the relative impedance through the fluidic medium 180 in the region/chamber 202 at each illuminated DEP electrode region 214a.

With the power source 212 activated, the foregoing DEP configuration creates an electric field gradient in the fluidic medium 180 between illuminated DEP electrode regions 214a and adjacent dark DEP electrode regions 214, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 214 at the inner surface 208 of the region/chamber 202 by changing light patterns 218 projected from a light source 216 into the microfluidic device 200. Whether the DEP forces attract or repel nearby micro-objects can depend on such parameters as the frequency of the power source 212 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 220 of illuminated DEP electrode regions 214a illustrated in FIG. 2F is an example only. Any pattern of the DEP electrode regions 214 can be illuminated (and thereby activated) by the pattern of light 218 projected into the microfluidic device 200, and the pattern of illuminated/activated DEP electrode regions 214 can be repeatedly changed by changing or moving the light pattern 218.

In some embodiments, the electrode activation substrate 206 can comprise or consist of a photoconductive material. In such embodiments, the inner surface 208 of the electrode activation substrate 206 can be featureless. For example, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 214 can be created anywhere and in any pattern on the inner surface 208 of the electrode activation substrate 206, in accordance with the light pattern 218. The number and pattern of the DEP electrode regions 214 thus need not be fixed, but can correspond to the light pattern 218. Examples of microfluidic devices having a DEP configuration comprising a photoconductive layer such as discussed above have been described, for example, in U.S. Pat. No. RE 44,711 (Wu, et al., originally issued as U.S. Pat. No. 7,612,355), each of which is incorporated herein by reference in its entirety.

In other embodiments, the electrode activation substrate 206 can comprise a substrate comprising a plurality of doped layers, electrically insulating layers (or regions), and electrically conductive layers that form semiconductor integrated circuits, such as is known in semiconductor fields. For example, the electrode activation substrate 206 can comprise a plurality of phototransistors, including, for example, lateral bipolar phototransistors, with each phototransistor corresponding to a DEP electrode region 214. Alternatively, the electrode activation substrate 206 can comprise electrodes (e.g., conductive metal electrodes) controlled by phototransistor switches, with each such electrode corresponding to a DEP electrode region 214. The electrode activation substrate 206 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 214 at the inner surface 208 of the electrode activation substrate 206 and the bottom electrode 204, and those electrical connections (i.e., phototransistors or electrodes) can be selectively activated and deactivated by the light pattern 218, as described above. When not activated, each electrical connection can have high impedance such that the relative impedance through the electrode activation substrate 206 (i.e., from the bottom electrode 204 to the inner surface 208 of the electrode activation substrate 206 which interfaces with the fluidic medium 180 in the region/chamber 202) is greater than the relative impedance through the fluidic medium 180 (i.e., from the inner surface 208 of the electrode activation substrate 206 to the top electrode 210 of the cover 110) at the corresponding DEP electrode region 214. When activated by light in the light pattern 218, however, the relative impedance through the electrode activation substrate 206 is less than the relative impedance through the fluidic medium 180 at each illuminated DEP electrode region 214, thereby activating the DEP electrode at the corresponding DEP electrode region 214 as discussed above. DEP electrodes that attract or repel micro-objects (not shown) in the fluidic medium 180 can thus be selectively activated and deactivated at many different DEP electrode regions 214 at the inner surface 208 of the electrode activation substrate 206 in the region/chamber 202 in a manner determined by the light pattern 218.

Examples of microfluidic devices having electrode activation substrates that comprise phototransistors have been described, for example, in U.S. Pat. No. 7,956,339 (Ohta et al.), the entire contents of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 210 is part of a first wall (or cover 110) of the enclosure 102, and the electrode activation substrate 206 and bottom electrode 204 are part of a second wall (or support structure 104) of the enclosure 102. The region/chamber 202 can be between the first wall and the second wall. In other embodiments, the electrode 210 is part of the second wall (or support structure 104) and one or both of the electrode activation substrate 206 and/or the electrode 210 are part of the first wall (or cover 110). Moreover, the light source 216 can alternatively be used to illuminate the enclosure 102 from below.

With the microfluidic device 200 of FIGS. 2E-2F having a DEP configuration, the motive module 162 can select a micro-object (not shown) in the fluidic medium 180 in the region/chamber 202 by projecting a light pattern 218 into the microfluidic device 200 to activate a first set of one or more DEP electrodes at DEP electrode regions 214a of the inner surface 208 of the electrode activation substrate 206 in a pattern (e.g., square pattern 220) that surrounds and captures the micro-object. The motive module 162 can then move the in situ-generated captured micro-object by moving the light pattern 218 relative to the microfluidic device 200 to activate a second set of one or more DEP electrodes at DEP electrode regions 214. Alternatively, the microfluidic device 200 can be moved relative to the light pattern 218.

In other embodiments, the microfluidic device 200 can have a DEP configuration that does not rely upon light activation of DEP electrodes at the inner surface 208 of the electrode activation substrate 206. For example, the electrode activation substrate 206 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 214, thereby creating a net DEP force on a micro-object (not shown) in region/chamber 202 in the vicinity of the activated DEP electrodes. Depending on such characteristics as the frequency of the power source 212 and the dielectric properties of the medium (not shown) and/or micro-objects in the region/chamber 202, the DEP force can attract or repel a nearby micro-object. By selectively activating and deactivating a set of DEP electrodes (e.g., at a set of DEP electrodes regions 214 that forms a square pattern 220), one or more micro-objects in region/chamber 202 can be selected and moved within the region/chamber 202. The motive module 162 in FIG. 1A can control such switches and thus activate and deactivate individual ones of the DEP electrodes to select, and move particular micro-objects (not shown) around the region/chamber 202. Microfluidic devices having a DEP configuration that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

As yet another example, the microfluidic device 200 can have an EW configuration, which can be in place of the DEP configuration or can be located in a portion of the microfluidic device 200 that is separate from the portion which has the DEP configuration. The EW configuration can be an OEW configuration or an electrowetting on dielectric (EWOD) configuration, both of which are known in the art. In some EW configurations, the support structure 104 has an electrode activation substrate 206 sandwiched between a dielectric layer (not shown) and the bottom electrode 204. The dielectric layer can comprise a hydrophobic material and/or can be coated with a hydrophobic material, as described below. For microfluidic devices 200 that have an EW configuration, the inner surface 208 of the support structure 104 is the inner surface of the dielectric layer or its hydrophobic coating.

The dielectric layer (not shown) can comprise one or more oxide layers. In some embodiments, each of the one or more oxide layers has a thickness of about 50 nm to about 250 nm (e.g., about 125 nm to about 175 nm). In certain embodiments, the dielectric layer may comprise a layer of oxide, such as a metal oxide (e.g., aluminum oxide or hafnium oxide). In certain embodiments, the dielectric layer can comprise a dielectric material other than a metal oxide, such as silicon oxide or a nitride. Regardless of the exact composition and thickness, the dielectric layer can have an impedance of about 10 kOhms to about 50 kOhms.

In some embodiments, the surface of the dielectric layer that faces inward toward region/chamber 202 is coated with a hydrophobic material. The hydrophobic material can comprise, for example, fluorinated carbon molecules. Examples of fluorinated carbon molecules can include perfluoro-polymers such as polytetrafluoroethylene (e.g., TEFLON®) or poly(2,3-difluoromethylenyl-perfluorotetrahydrofuran) (e.g., CYTOP™). Molecules that make up the hydrophobic material can be covalently bonded to the surface of the dielectric layer. For example, molecules of the hydrophobic material can be covalently bound to the surface of the dielectric layer by means of a linker such as a siloxane group, a phosphonic acid group, or a thiol group. Thus, in some embodiments, the hydrophobic material can comprise alkyl-terminated siloxane, alkyl-termination phosphonic acid, or alkyl-terminated thiol. The alkyl group can be long-chain hydrocarbons (e.g., having a chain of at least 10 carbons, or at least 16, 18, 20, 22, or more carbons). Alternatively, fluorinated (or perfluorinated) carbon chains can be used in place of the alkyl groups. Thus, for example, the hydrophobic material can comprise fluoroalkyl-terminated siloxane, fluoroalkyl-terminated phosphonic acid, or fluoroalkyl-terminated thiol. In some embodiments, the hydrophobic coating has a thickness of about 10 nm to about 50 nm. In other embodiments, the hydrophobic coating has a thickness of less than 10 nm (e.g., less than 5 nm, or about 1.5 to 3.0 nm). Further details of the hydrophobic material and its preparation are found in U.S. Patent Application Publication No. 2017/0173580 (Lowe, Jr. et al.), the disclosure of which is herein incorporated by reference in its entirety.

In some embodiments, the cover 110 of a microfluidic device 200 having an electrowetting configuration is coated with a hydrophobic material (not shown) as well. The hydrophobic material can be the same hydrophobic material used to coat the dielectric layer of the support structure 104, and the hydrophobic coating can have a thickness that is substantially the same as the thickness of the hydrophobic coating on the dielectric layer of the support structure 104. Moreover, the cover 110 can comprise an electrode activation substrate 206 sandwiched between a dielectric layer and the top electrode 210, in the manner of the support structure 104. The electrode activation substrate 206 and the dielectric layer of the cover 110 can have the same composition and/or dimensions as the electrode activation substrate 206 and the dielectric layer of the support structure 104. Thus, the microfluidic device 200 can have two electrowetting surfaces.

In some embodiments, the electrode activation substrate 206 can comprise a photoconductive material, such as described above. Accordingly, in certain embodiments, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. Alternatively, the electrode activation substrate 206 can comprise electrodes (e.g., conductive metal electrodes) controlled by phototransistor switches, as described above. Microfluidic devices having an opto-electrowetting configuration are known in the art and/or can be constructed with electrode activation substrates known in the art. For example, U.S. Pat. No. 6,958,132 (Chiou et al.), the entire contents of which are incorporated herein by reference, discloses opto-electrowetting configurations having a photoconductive material such as a-Si:H, while U.S. Pat. No. 9,403,172 (Short, et al.), referenced herein, discloses electrode activation substrates having electrodes controlled by phototransistor switches.

The microfluidic device 200 thus can have an opto-electrowetting configuration, and light patterns 218 can be used to activate photoconductive EW regions or photoresponsive EW electrodes in the electrode activation substrate 206. Such activated EW regions or EW electrodes of the electrode activation substrate 206 can generate an electrowetting force at the inner surface 208 of the support structure 104 (i.e., the inner surface of the overlaying dielectric layer or its hydrophobic coating). By changing the light patterns 218 (or moving microfluidic device 200 relative to the light source 216) incident on the electrode activation substrate 206, droplets (e.g., containing an aqueous medium, solution, or solvent) contacting the inner surface 208 of the support structure 104 can be moved through an immiscible fluid (e.g., an oil medium) present in the region/chamber 202.

In some embodiments, the microfluidic devices 200 can have an EWOD configuration, and the electrode activation substrate 206 can comprise selectively addressable and energizable electrodes that do not rely upon light for activation. The electrode activation substrate 206 thus can include a pattern of such EW electrodes. The pattern, for example, can be an array of substantially square EW electrodes arranged in rows and columns. Alternatively, the pattern can be an array of substantially hexagonal EW electrodes that form a hexagonal lattice. Regardless of the pattern, the EW electrodes can be selectively activated (or deactivated) by electrical switches (e.g., transistor switches in a semiconductor substrate). By selectively activating and deactivating EW electrodes in the electrode activation substrate 206, droplets (not shown) contacting the inner surface 208 of the overlaying dielectric layer or its hydrophobic coating can be moved within the region/chamber 202. The motive module 162 in FIG. 1A can control such switches and thus activate and deactivate individual EW electrodes to select and move particular droplets around region/chamber 202. Microfluidic devices having a EWOD configuration with selectively addressable and energizable electrodes are known in the art and have been described, for example, in U.S. Pat. No. 8,685,344 (Sundarsan et al.), the entire contents of which are incorporated herein by reference.

In some embodiments, the microfluidic device may include both a dielectrophoresis electrode activation substrate and an electrowetting electrode activation substrate. Further details of such dual motive systems may be found in U.S. Patent Application Publication Nos. 2015/0306598 (Khandros et al.), 2015/0306599 (Khandros et al.), and 2017/0173580 (Lowe, Jr. et al.), each of which disclosures are herein incorporated by reference in its entirety.

Regardless of whether the microfluidic device 200 has a dielectrophoretic electrode activation substrate, an electrowetting electrode activation substrate or a combination of both a dielectrophoretic and an electrowetting activation substrate, a power source 212 can be used to provide a potential (e.g., an AC voltage potential) that powers the electrical circuits of the microfluidic device 200. The power source 212 can be the same as, or a component of, the power source 192 referenced in FIG. 1A. Power source 212 can be configured to provide an AC voltage and/or current to the top electrode 210 and the bottom electrode 204. For an AC voltage, the power source 212 can provide a frequency range and an average or peak power (e.g., voltage or current) range sufficient to generate net DEP forces (or electrowetting forces) strong enough to select and move individual micro-objects (not shown) in the region/chamber 202, as discussed above, and/or to change the wetting properties of the inner surface 208 of the support structure 104 (i.e., the dielectric layer and/or the hydrophobic coating on the dielectric layer) in the region/chamber 202, as also discussed above. Such frequency ranges and average or peak power ranges are known in the art. See, e.g., U.S. Pat. No. 6,958,132 (Chiou et al.), U.S. Pat. No. RE44,711 (Wu, et al., originally issued as U.S. Pat. No. 7,612,355), and U.S. Patent Application Publication Nos. 2014/0124370 (Short, et al.), 2015/0306598 (Khandros et al.), 2015/0306599 (Khandros et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can be also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger et al.), which is incorporated herein by reference in its entirety. Magnetic forces may be employed to move micro-objects including paramagnetic materials, which can include magnetic micro-objects attached to or associated with a biological micro-object. Alternatively or additionally, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz et al.), which is incorporated herein by reference in its entirety.

In some embodiments, DEP and/or EW forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP and/or EW forces can be applied prior to the other forces. In other embodiments, the DEP and/or EW forces can be applied after the other forces. In still other instances, the DEP and/or EW forces can be applied at the same time as the other forces or in an alternating manner with the other forces.

System.

Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂. The media module 160 may also control the temperature of an enclosure of the media source, for example, to provide feeder cells in the media source with proper temperature control. For example, in some embodiments media module 160 stops the flow of media 180 in the flow path 106 and through the enclosure 102 prior to the optional tilting module 166 causing the support structure 190 to tilt the microfluidic device 100 to a desired angle of incline.

Motive Module.

The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configuration may include one or more electrodes that apply a non-uniform electric field in the microfluidic circuit 120 sufficient to exert a dielectrophoretic force on micro-objects in the microfluidic circuit 120. An OET configuration may include photoactivatable electrodes to provide selective control of movement of micro-objects in the microfluidic circuit 120 via light-induced dielectrophoresis. An EW or OEW configuration may include one or more electrodes (photoactivatable, in the case of OEW) that apply an electric field in the microfluidic circuit 120 sufficient to modify the wetting properties of liquid droplets in the microfluidic circuit 120.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g. for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g. a high pressure mercury lamp) or a Xenon arc lamp. The imaging device may further include a microscope (or an optical train), which may or may not include an eyepiece.

Support Structure.

System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e. at 0° relative to x- and y-axes), a vertical orientation (i.e. at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) at 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal).

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e. an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 10°, about 5°, about 4°, about 3° or less than about 2° relative to the x-axis (horizontal), thereby placing the sequestration pens at a lower potential energy relative to the flow path.

Tilting may provide gravitational forces which can move a micro-object into or out of a sequestration pen. The optional tilting module 166 can control the tilting motions of the microfluidic device 100. Alternatively, or in addition, the tilting module 166 can control the tilting rate and timing to optimize transfer of micro-objects to the one or more sequestration pens via gravitational forces. The tilting module 166 can be communicatively coupled with the imaging module 164 to receive data describing the motion of micro-objects and/or droplets of medium in the microfluidic circuit 120. Using this data, the tilting module 166 may adjust the tilt of the microfluidic circuit 120 in order to adjust the rate at which micro-objects and/or droplets of medium move in the microfluidic circuit 120. The tilting module 166 may also use this data to iteratively adjust the position of a micro-object and/or droplet of medium in the microfluidic circuit 120. Further examples of the use of gravitational forces induced by tilting are described in U.S. Pat. No. 9,744,533 (Breinlinger et al.), the contents of which are herein incorporated by reference in its entirety.

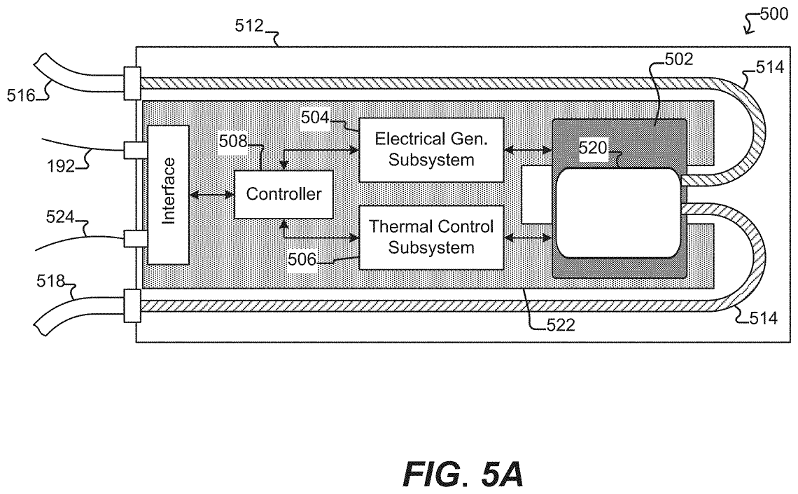

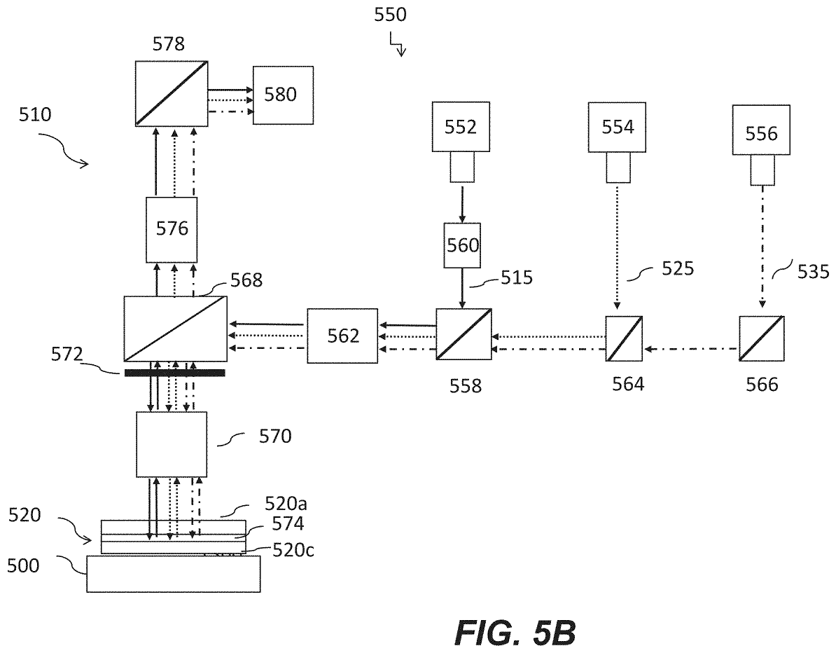

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically-actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 5302 mounted on PCBA 322, as well.

In some embodiments, the nest 500 can comprise an electrical signal generation subsystem 504 comprising a Red Pitaya™ waveform generator/oscilloscope unit (“Red Pitaya unit”) and a waveform amplification circuit that amplifies the waveform generated by the Red Pitaya unit and passes the amplified voltage to the microfluidic device 520. In some embodiments, the Red Pitaya unit is configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 3A the controller 308 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). The Peltier thermoelectric device can have a first surface configured to interface with at least one surface of the microfluidic device 520. The cooling unit can be, for example, a cooling block (not shown), such as a liquid-cooled aluminum block. A second surface of the Peltier thermoelectric device (e.g., a surface opposite the first surface) can be configured to interface with a surface of such a cooling block. The cooling block can be connected to a fluidic path 314 configured to circulate cooled fluid through the cooling block. In the embodiment illustrated in FIG. 5A, the support structure 500 comprises an inlet 516 and an outlet 518 to receive cooled fluid from an external reservoir (not shown), introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

In some embodiments, the nest 500 can include a thermal control subsystem 506 with a feedback circuit that is an analog voltage divider circuit (not shown) which includes a resistor (e.g., with resistance 1 kOhm+/−0.1%, temperature coefficient+/−0.02 ppm/C0) and a NTC thermistor (e.g., with nominal resistance 1 kOhm+/−0.01%). In some instances, the thermal control subsystem 506 measures the voltage from the feedback circuit and then uses the calculated temperature value as input to an on-board PID control loop algorithm. Output from the PID control loop algorithm can drive, for example, both a directional and a pulse-width-modulated signal pin on a Pololu™ motor drive (not shown) to actuate the thermoelectric power supply, thereby controlling the Peltier thermoelectric device.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical System.

FIG. 5B is a schematic of a system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560, includes mirror, the modulator can have a plurality of mirrors. Each mirror of the plurality of mirrors can have a size of 5 microns×5 microns, 6 microns×6 microns, 7 microns×7 microns, 8 microns×8 microns, 9 microns×9 microns, 10 microns×10 microns, or any values therebetween. The structured light modulator 560 can include an array of mirrors (or pixels) that is 2000×1000, 2580×1600, 3000×2000, or any values therebetween. For a mirror size of 7.6 microns×7.6 microns, the structured light modulator 560 can have the dimensions of 15.2 mm×7.6 mm, 19.6 mm×12.2 mm, 22.8 mm×15.2 mm, or any values therebetween. The active area of a structured light modulator 560 can be at least 10 mm×10 mm (e.g., at least 10.5 mm×10.5 mm, 11 mm×11 mm, 11.5 mm×11.5 mm, 12 mm×12 mm, 12.5 mm×12.5 mm, 13 mm×13 mm, 13.5 mm×13.5 mm, 14 mm×14 mm, 14.5 mm×14.5 mm, 15 mm×15 mm, or greater). In some embodiments, only a portion of an illumination area of the structured light modulator 560 is used. For example, 50%, 60%, 80% or any values therebetween of the illumination area of the structured light modulator 560 is used. The structured light modulator 560 can transmit the selected subset of light to a first dichroic beam splitter 558, which can reflect this light to a first tube lens 562.

The first tube lens 562 can be configured to have a large field of view that is larger than the illumination area of the structured light modulator 560. The first tube lens 562 can be configured to capture all light beams from the structured light modulator 560. The first tube lens 562 can have a large clear aperture, for example, a diameter larger than 40 mm, 41 mm, 42 mm, 43 mm, 44 mm, 45 mm, 46 mm, 47 mm, 48 mm, 49 mm, 50 mm, etc. Thus, the first tube lens 5621 can have an aperture that is large enough to capture all (or substantially all) of the light beams emanating from the structured light modulator.

The structured light 515, having a wavelength of about 400 nm to about 710 nm, may alternatively or in addition, provide fluorescent excitation illumination to the microfluidic device. In some embodiments, the structured light 515 may have a wavelength of about 400 nm to about 650 nm, about 400 nm to about 600 nm, about 400 nm to about 550 nm, about 400 nm to about 500 nm, about 450 nm to about 710 nm, about 450 nm to about 600 nm, or about 450 nm to about 550 nm.

The second light source 554 may provide unstructured brightfield illumination. The brightfield illumination light 525 may have any suitable wavelength, and in some embodiments, may have a wavelength of about 400 nm to about 760 nm. In some embodiments, the brightfield illumination light 525 may have a wavelength of more than about 530 nm and less than about 760 nm, more than about 600 nm and less than about 750 nm, or about 650 nm and less than about 750 nm. In some embodiments, the brightfield illumination light 525 may have a wavelength of about 700 nm, about 710 nm, about 720 nm, about 730 nm, about 740 nm, or about 750 nm. The second light source 554 can transmit light to a second dichroic beam splitter 564 (which also may receive light 535 from the third light source 556), and the second light, brightfield illumination 525, may be transmitted therefrom to the first dichroic beam splitter 558. The second light, brightfield illumination 525, may then be transmitted from the first beam splitter 558 to the first tube lens 562.

The third light source 556 can transmit light through a matched pair relay lens (not shown) to a mirror 566. The third light illumination 535 may therefrom be reflected to the second dichroic beam splitter 5338 and be transmitted therefrom to the first beam splitter 5338, and onward to the first tube lens 5381. The third illumination light 535, which may be a laser, may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The laser illumination 535 may have any suitable wavelength. In some embodiments, the laser illumination 535 may have a wavelength of about 350 nm to about 900 nm, about 370 nm to about 850 nm, about 390 nm to about 825 nm, about 400 nm to about 800 nm, about 450 nm to about 750 nm, or any value therebetween. In some embodiments, the laser illumination 535 may have a wavelength of about 700 nm, about 710 nm, about 720 nm, about 730 nm, about 740 nm, about 750 nm, about 760 nm, about 770 nm, about 780 nm, about 790 nm, about 800 nm, about 810 nm or more.

The light from the first, second, and third light sources (552, 554, 5560) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500 can be configured to hold the microfluidic device 520 and provide electrical connection to the enclosure, as described in FIG. 5A. The nest 500 can be integrated with the optical apparatus 510 and be a part of the apparatus 510. The nest 500 can be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective.

The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4×, 10×, 20×.

In some embodiments, the objective lens 570 may be configured so that light emerging from a rear aperture of the objective lens 570 may be focused to infinity, and the second tube lens 576 is configured to form an image of the micro-objects in the plurality of sequestration pens within the sample plane 574 at a focal plane of the tube lens 576. Light beams exiting the infinity-focused objective lens 570 can be configured to be collimated, such that the beam-splitter 568 and other components can be easily introduced into the imaging path of the optical apparatus 510 without the introduction of spherical aberration or modification of a working distance of the objective lens 570.

In some embodiments, the first tube lens 562 can have a focal length of about 155 mm or about 162 mm and the second tube lens 576 can have a focal length of about 180 mm. In some other embodiments, the first tube lens 562 can have a focal length of about 180 mm and the second tube lens 576 can have a focal length of about 200 mm.

The objective lenses 570 may be configured to image at least a portion of the plurality of sequestration pens in the sample plane 574 of the microfluidic device 520 within a field of view. The field of view, for example, can be larger than 10 mm×10 mm, 11 mm×11 mm, 12 mm×12 mm, 13 mm×13 mm, 14 mm×14 mm, 15 mm×15 mm, etc.

Modes of Illumination.

In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520. Alternatively, optically actuated electrowetting electrodes in the substrate 520c may be selectively activated by a shifting plurality of illumination patterns to generate electrowetting forces to move droplets within the enclosure of the microfluidic device 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots into each of the plurality of sequestration pens in the sample plane 574 within the field of view. For example, each of the plurality of illumination spots can have a size of about 10 microns×30 microns, 30 microns×60 microns, 40 microns×40 microns, 40 microns×60 microns, 60 microns×120 microns, 80 microns×100 microns, 100 microns×140 microns and any values there between. For example, each of the plurality of illumination spots can have an area of about 4000 to about 10000, 5000 to about 15000, 7000 to about 20000, 8000 to about 22000, 10000 to about 25000 square microns and any values there between.

In some embodiments, the optical apparatus 510 can be configured to perform confocal imaging. For example, the structured light modulator 560 can be configured to generate a thin strip that can scan through the plurality of sequestration pens in the sample plane 574 within the field of view to reduce out-of-focus light to reduce overall noise. For another example, the structured light modulator 560 can be configured to generate a plurality of illuminations spots within diffraction limits. For another example, the structured light modulator 560 can be configured to move along an optical axis of the optical apparatus 510 to obtain a plurality of images along the optical axis, the plurality of images along the optical axis can be combined to reconstruct 3 dimensional images of the micro-objects in the plurality of sequestration pens in the sample plane 574 in the microfluidic apparatus 520.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger et al.); and International Application Serial No. PCT/US2018/055918 (Lionberger et al.).

Further details of the features of optical apparatuses suitable for use within a system for observing and manipulating micro-objects within a microfluidic device, as described herein, may be found in WO2018/102747 (Lundquist et al), the disclosure of which is herein incorporated by reference in its entirety.

### EXPERIMENTAL

System and Microfluidic Device:

The foregoing experiments were performed using an OptoSelect™ microfluidic (or nanofluidic) device manufactured by Berkeley Lights, Inc. and controlled by an optical instrument which was also manufactured by Berkeley Lights, Inc. The instrument included: a mounting stage for the microfluidic device coupled to a temperature controller; a pump and fluid medium conditioning component; an optical train including a camera and a structured light source suitable for activating phototransistors within the microfluidic device; and software for controlling the instrument, including performing image analysis and automated detection and repositioning of micro-objects. The OptoSelect device included a substrate configured with OptoElectroPositioning (OEP™) technology, which provides a phototransistor-activated dielectrophoresis (DEP) force. The device also included a plurality of microfluidic channels, each having a plurality of NanoPen™ chambers (or sequestration pens) fluidically connected thereto. The volume of each sequestration pen was around 1×10⁶ cubic microns. The microfluidic device included conditioned interior surfaces, which are described in U.S. Patent Application Publication No. US2016/0312165 (Lowe, Jr., et al.), International Patent Application Publication WO2017/205830 (Lowe, Jr., et al.), and International Patent Application Publication WO2019/01880 (Beemiller et al.), each of which disclosures is herein incorporated by reference in its entirety.

Priming regime. 250 microliters of 100% carbon dioxide was flowed in at a rate of 12 microliters/sec. This was followed by 250 microliters of a priming medium composed as follows: 1000 ml Iscove's Modified Dulbecco's Medium (ATCC® Catalog No. 30-2005), 200 ml Fetal Bovine Serum (ATCC® Cat. #30-2020), 10 ml penicillin-streptomycin (Life Technologies® Cat. #15140-122), and 10 mL Pluronic F-127 (Life Tech Catalog No. 50-310-494). The final step of priming included 250 microliters of the priming medium, flowed in at 12 microliters/sec. Introduction of the culture medium follows.

Perfusion regime. The perfusion method was either of the following two methods:

1. Perfuse at 0.01 microliters/sec for 2 h; perfuse at 2 microliters/sec for 64 sec; and repeat.

2. Perfuse at 0.02 microliters/sec for 100 sec; stop flow 500 sec; perfuse at 2 microliters/sec for 64 sec; and repeat.

Culture medium: (RPMI 1640 (Gibco), 10% FBS (Seradigm), 2 ug/mL CpG (Invivogen), 1 ug/mL IL-2 (Peprotek), 5 ng/mL IL-4 (Peprotek), 10 ng/mL IL-6 (Peprotek), 10 ng/mL IL-21 (Peprotek), and 10 ng/mL BAFF (Peprotek)).

### Example 1. Single End Random Fragmentation Sequencing Using 3′ Anchored Sequencing of Variable 5′ Regions. Fragmentation by Tagmentation of Amplified DNA

FIG. 7 represents a schematic of the experiment from capture of RNA to production of the indexed and fragmented sequencing library 742.

RNA Isolation.

Single cells (Human B cells) were exported from the microfluidic device in 5 microliter volumes and added to 5 microliters of TCL buffer (Qiagen, Cat. #1070498). RNAClean XP SPRI beads (Beckman Coulter #A63987) were brought to room temperature and 10 microliters of the bead mixture (1× volume) was added to each well. (1× volume SPRI beads showed higher RNA recovery compared to standard 1.8× to 2.2×.)

Lysate and bead mixture were incubated at room temperature for 15 min. This extended period of incubation provided improved binding of released RNA. The plate was subsequently transferred on to a 96-well plate magnet (MagWell™ Magnetic Separator 96, Cat. #57624) and incubated for 5 min. Supernatant was carefully removed and ethanol wash was performed by adding 100 microliters of 80% ethanol (Sigma Cat. #E7023, prepared fresh). After approximately 30 sec., ethanol was aspirated and the ethanol wash was repeated. After the final aspiration the plate was removed from the 96-well plate magnet and the beads were dried for 5 min., where the beads contain a mixture 710 of captured RNA as shown in FIG. 7.

cDNA Synthesis.

The plate was transferred to 4° C. and the beads were resuspended in 4 microliters of “RT mix 1”: containing 0.8 microliters RNase free water (Ambion Cat no AM9937); 1 microliter of 1:5M External RNA Controls Consortium (ERCC) control RNA (ThermoFisher Scientific Cat. #4456740); 1 microliter of dNTPs (10 mM each, NEB, #N0447L); 1 microliter of biotin-dTVI RNA capture/priming oligonucleotide (biot-AAGCAGTGGTATCAACGCAGAGTACTTTTTTTTTTTTTTTTTTTTTTTTTTTTTTVI, (SEQ ID NO. 97); and 0.2 microliters of RNaseOUT (4 U/microliter, Life Technologies Cat. #107777-019). The 3′ inosine of the capture sequence of the biotin-dTVI RNA capture/priming oligonucleotide provided increased binding to released RNA as inosine may bind to any natural nucleotide. The capture sequence having a 3′ inosine can provide better capture of released RNA than a capture sequence including a final “N” nucleotide, which may bind to the mRNA only 25% of the time. The ERCC RNA controls provided an internal RT control and also provided carrier RNA improving reverse transcription efficiency. The plate was incubated at 72° C. for 5 min and immediately transferred to 4° C. 4 microliters of “RT mix 2” containing 1 microliter of betaine (5M, Sigma Cat. #B030075VL); 1.5 microliter of 5×RT mix (Thermo, #EP0753), 0.5 microliters of biotinylated-Template Switching Oligonucleotide (biot_TSO; biot-GTGGTATCAACGCAGAGTACACGACGCTCTTCCGATCTrGrGrG) (SEQ ID NO. 98), 0.5 microliters of 120 mM MgCl₂ (125 mM, Life Technologies Cat. #AM9530G), 0.4 microliters of RNase OUT and 0.1 microliter of Maxima RNaseH minus reverse transcriptase (200 U/microliter, Thermo Fisher Cat. #EP0753) was added to each well. Following the additional of the “RT mix 2”, reverse transcription was carried out at 42° C. for 90 min followed by 10 cycles of: 50° C. for 2 min/42° C. for 2 min. The last thermal cycle was followed by heat inactivation at 75° C. for 15 min. The bio_TSO is a nested TSO where the addition of 18 nucleotides (underlined section above) downstream of the P1 primer helps to amplify BCR specific amplicons. A mixture 720 of cDNA is the product of the reverse transcription.

Whole mRNA Amplification (e.g., Full Length Sample DNA Sequence).

Following cDNA synthesis, the export plate was centrifuged at 200 g for 5 min and 17 microliters of PCR mix containing 12.5 microliters 2× Kapa Hi Fi HotStart ReadyMix (Roche Cat. #KK2602), 1 microliter of P1 primer (biot_P1, biot-AAGCAGTGGTATCAACGCAGAGT) (SEQ ID NO. 99) and 3.5 microliters of nucleotide-free water (Ambion Cat. #AM9937) was added and PCR was carried out at 98° C. for 3 min followed by 20 cycles of: 98° C. for 15 s, 65° C. for 30 s, 72° C. for 5 min, and a final extension of 5 min at 72° C. was performed. The final extension period was long enough for the polymerase to amplify long cDNA molecules (greater than 2 kb).

PCR Clean-Up.

25 microliters (1× volume) DNAClean SPRI beads (Beckman Coulter, Cat. #A62881) were added to each well and mixed well, removing primer-dimer and short degraded RNA products which could contaminate the downstream amplification. The mixture was incubated for 10 min at room temperature. Following incubation, the plate was placed on the well plate magnet for 5 min. Supernatant was carefully removed and ethanol wash was performed by adding 100 microliters of 80% ethanol (prepared fresh). After approximately 30 sec, ethanol was aspirated. The ethanol wash procedure was repeated once. After the final aspiration the export well plate was removed from the well plate magnet and the beads were dried for 5 min.

The amplified whole mRNA product (e.g. amplification product containing the full-length sample DNA sequence) was quantified using a high sensitivity DNA quantitation kit such as Qubit dsDNA HS reagent.

Tagmentation Reaction:

To 2 ul at 250 pg of pre-amplified quantified cDNA reaction was added 4 ul of 2× tagment DNA buffer (TD), 2 ul of amplification tagment mix (ATM) (Illumina Catalog #FC-131-1024) to a final volume of 8 ul. The solution was incubated for 5 min at 55° C. followed by cooling to 10° C. Immediately after the thermocycler reached 10° C., 2 ul of neutralization tagment buffer (NT) (Illumina Catalog No. FC-131-1024) was added to tagmented reaction mixture 730 and vortexed. The tube was spun down and incubated at room temperature for 5 min. After incubation the whole volume was used for limited-cycle gene enrichment PCR.

SERF-Seq PCR Amplification—Heavy Chain:

To the 10 ul of tagmented product, was added 1 ul of Illumina index primer (I5) (Illumina Catalog No. FC-131-1024), 1 ul of P7-Hc (heavy chain gene-specific primer with Illumina compatible (P7) overhang), 6 ul of Nextera PCR master Mix (NPM) (Illumina Catalog #FC-131-1024) and 2 ul of nuclease-free H₂O. PCR was performed as follows: 72° C. for 3 min, 95° C. for 30 s followed by 15 cycles of 98° C. for 10 s; 55° C. for 30 s; 72° C. for 30 s; followed by a final extension of 5 min at 72° C. Hold at 10° C.

Primer P7-Hc:
(SEQ ID NO. 100)
ACTGGAGTTCAGACGTGTGCTCTTCCGATCTggaagacsgatgggccatg
gt.

SERF-Seq PCR amplification—Light Chain:

To the 10 ul of tagmented product, was added 1 ul of Illumina index primer (I5), from Illumina Catalog No. FC-131-1024, 1 ul of P7-Kc/lambda c (gene-specific primer with Illumina compatible (P7) overhang), 6 ul of Nextera PCR master Mix (NPM), from Illumina, Catalog No. FC-131-1024, and 2 ul of nuclease-free H₂O. PCR was performed as follows: 72° C. for 3 min, 95° C. for 30 s followed by 15 cycles of 98° C. for 10 s; 55° C. for 30 s; 72° C. for 30 s; followed by a final extension of 5 min at 72° C. Hold at 10° C.

Primer P7 - Kappa c:
(SEQ ID NO. 101)
ACTGGAGTTCAGACGTGTGCTCTTCCGATCTtgaagacagatg
gtgcagccacagt
Primer P7 - lambda c1:
(SEQ ID NO. 102)
ACTGGAGTTCAGACGTGTGCTCTTCCGATCTacagagtgacMg
tggggttggcctt
Primer P7 - lambda c2:
(SEQ ID NO. 103)
ACTGGAGTTCAGACGTGTGCTCTTCCGATCTacagagtgaccg
aKggggcagcctt

Post-PCR Clean-Up:

AMPure clean up was performed for each reaction with 1× volume DNAClean SPRI beads (Beckman Coulter, Catalog no A62881) added to the pooled product. The mixture was incubated for 10 min at room temperature. Following incubation, the wellplate was placed on a magnet for 5 min to pull down the beads carrying the amplified product. Supernatant was carefully removed and ethanol wash was performed by adding 100 ul of 80% ethanol (prepared fresh). Approximately after 30 s ethanol was aspirated and the ethanol wash was performed 1 more time. After the final aspiration the plate was removed from the magnet and the beads were dried for 5 min. DNA was eluted from the dried beads with 15 ul of nuclease-free H₂O.

Barcoding Library PCR:

The product from the SERF-seq PCR required barcoding and extension to make it compatible with Illumina sequencing primers. The following PCR added the necessary extension to the gene specific end and also barcoded each sample (in this example, a hexamer barcode sequence was used, indicated in the adapter sequence as “nnnnnn” (SEQ ID NO. 104). Using 2 ul from the SERF-seq PCR as template DNA, PCR2 was setup in the following order: 5 ul of 2× Kapa high-fidelity master mix, 0.2 ul of I5 index primer, 1 ul of barcoded P7 extension and 1.8 ul of nuclease free water was added for a total of 10 ul reaction volume. Setup PCR cycling conditions at 98° C. for 3 min followed by 12 cycles of 98° C. for 20 s; 65° C. for 30 s; 72° C. for 30 s; and followed by final extension at 72° C. for 5 min. This provides a DNA library having a Barcoded P7 extension having the following sequence: CAAGCAGAAGACGGCATACGAGATnnnnnnGTGACTGGAGTTCAGACGTGT (SEQ ID NO. 105)

Pooled Amplicon Cleanup:

All reactions were combined and purified with 1× volume DNAClean SPRI beads (Beckman Coulter, Catalog No. A62881) added to the pooled product. The mixture was incubated for 10 min at room temperature. Following incubation, the plate was placed on magnet for 5 min. Supernatant was carefully removed and ethanol wash was performed by adding 100 ul of 80% ethanol (prepared fresh). Approximately after 30 s ethanol was aspirated and the ethanol wash was performed 1 more time. After the final aspiration the plate was removed from the magnet and the beads were dried for 5 min. DNA was eluted from the dried beads with 15 ul of nuclease-free H2O.

Library Quality Check:

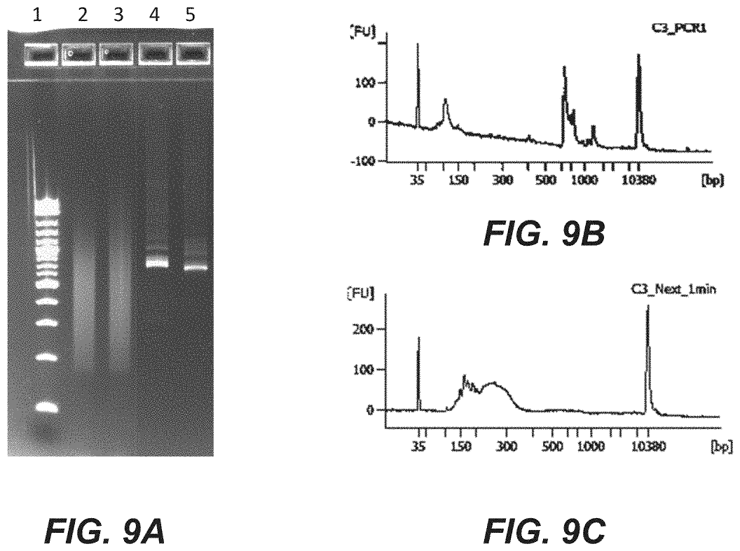

1 ul of cleaned PCR product was run on a 2% Agarose gel. A photograph of the gel is shown in FIG. 9A, and shows the lanes as follows:

Lane 1: 100 bp DNA ladder (NEB: N3231).

Lane 2: SERF-seq amplified human Hc.

Lane 3: SERF-seq amplified human Kc.

Lane 4: Human Hc amplified using forward primer.

Lane 5: Human Kc amplified using forward primer.

1 ul of amplified DNA was run on Agilent Bioanalyzer 2100 instrument, and the graphical results were as shown in FIG. 9B showing large fragments having a size above 500 bp to over 1000 bp. For BCR chains amplified using the constant region anchor and tagmentation process of this experiment, the graph of product DNA fragments shows fragments having a size ranging from 50 bp to about 300 bp as shown in FIG. 9C.

Next Generation Sequencing:

All libraries were sequenced on Illumina Miseq 2×75 bp according to standard procedures.

Post Sequencing Assembly:

Denovo assembly of the genes can be done from the 5′ end and staggering along all the sequences until full constant region sequence is achieved.

As shown in Table 2, the representative sequences from Miseq that stagger along the human lambda chain starting with primer sequence and below is the full-length reconstructed sequence from reading 75 bp contigs. The italicized sequences are the portions of the fragment read permitting tiling and reconstruction of the full sequence.

TABLE 2
Sequence reads and tiling.

@M03786:42:000000000-AR25C:1:1110:17715:10263
1:N:0:100
GGCCATTATGGCCGGGGGTAGCTCAGGAAGCAGAGCCTGGAGCAT
CTCCACTAT- [symbol] [symbol]
(SEQ ID NO. 106)
@M03786:42:000000000-AR25C:1:1110:15275:10307
1:N:0:100 [symbol] [symbol]
ACCCTCCTCGCTCACTGCACA
GGTTCTTG- [symbol] [symbol]
(SEQ ID NO. 107)
@M03786:42:000000000-AR25C:1:1110:11458:10336
1:N:0:100 [symbol] [symbol] [symbol]
CACTCTGTGTCGGCGTCT
CCGGGGAAGA- [symbol] [symbol]
(SEQ ID NO. 108)
@M03786:42:000000000-AR25C:1:1110:14985:10662
1:N:0:100 [symbol] [symbol] [symbol]
AGCAGTGGAAACATTGCCACCAAC
TATGTGCA- [symbol] [symbol] [symbol]
(SEQ ID NO. 109)
@M03786:42:000000000-AR25C:1:1110:26633:10697
1:N:0:100 [symbol] [symbol] [symbol]
CAGTCCCCCCACCACTATGATCTATG
AAAATA- [symbol] [symbol] [symbol]
(SEQ ID NO. 110)
@M03786:42:000000000-AR25C:1:1110:27399:10862
1:N:0:100 [symbol] [symbol] [symbol]
GATCGGGTCTCTGGCTCCATCGA
CAGCTC- [symbol] [symbol] [symbol]
(SEQ ID NO. 111)
@M03786:42:000000000-AR25C:1:1110:21050:11052
1:N:0:100 [symbol] [symbol] [symbol]
CTCTGGACTGAGTCCTGAG
GACGAG- [symbol] [symbol] [symbol]
(SEQ ID NO. 112)
@M03786:42:000000000-AR25C:1:1110:6314:11053
1:N:0:100 [symbol] [symbol] [symbol]
AGGGCA-

[symbol]

[symbol]

[symbol]

[symbol]

[symbol]

(SEQ ID NO. 113)
@M03786:42:000000000-AR25C:1:1110:19934:11920
1:N:0:100

[symbol]

[symbol]

[symbol]

[symbol]

[symbol]

AGCCCAAGGCTGCCCCCTCGGTCACTCTGT (SEQ ID NO. 114)

Reconstructed lambda chain from SERF-seq PCR is as follows, where italicized segments alternate with non-italicized to illustrate the fragment reconstruction of the lambda chain sequence:

(SEQ ID NO. 115)
TAGCTCAGGAAGCAGAGCCTGGAGCATCTCCACTATGGCCTGGGCTC
CACTACTTCTCACCCTCCTCGCTCACTGCACAGGTTCTTGGGCCAAT
TTTATGCTGACTCAGCCGCACTCTGTGTCGGCGTCTCCGGGGAAGAC
GGTAACCATCTCCTGCTCCCGCAGCAGTGGAAACATTGCCACCAACT
ATGTGCAGTGGTACCAGCAGCGCCCGGGCAGTCCCCCCACCACTATG
ATCTATGAAAATAGTCAAAGGCCTTCTGGAGTCCCTGATCGGGTCTC
TGGCTCCATCGACAGCTCCTCCAATTCTGCCTCCCTCACCATCTCTG
GACTGAGTCCTGAGGACGAGGCTGACTACTACTGTCAGTCCTATGAG
GGCAGTACTGTGGTTTTCGGCGGAGGGACCAAGCTGACCGTCCTAAG
TCAGCCCAAGGCTGCCCAACGGT

### Example 2. Single End Random Fragmentation Sequencing Using 3′ Anchored Sequencing of Variable 5′ Regions. SERFr-Seq: Single End Random Fragmentation by Chemical Fragmentation of RNA Intermediates

Schematic representations are shown in FIGS. 10A-C.

Human primary T cells are used similarly as in Example 1 above to construct a cDNA library containing TCR sequences.

cDNA Amplification.

Full length TCR sequences 1010 are amplified to contain a 5′ T7 RNA polymerase promoter sequence. Amplification is performed (amplification complex 1015) using primers 3 (1006) and 4a/4b (1008), as shown in Table 3. Primer 3 contains the T7 RNA polymerase promoter, which is required for in vitro transcription of the RNA. The 5′-phosphate modification of amplicons 1020 is not required for the in vitro transcription of RNA, but helps to prevent artifactual amplification in the PCR reaction. Kapa Hotstart PCR master mix may be used, but the method may also be practiced using other DNA polymerases and other PCR master mixes, with adjustment of conditions.

The alpha chains are amplified using an annealing temperature of 70° C. and the beta chains are amplified using an annealing temperature of 64° C. Both chains are amplified for 30 cycles. Fewer PCR cycles may be used in the amplification in some case to mitigate putative PCR errors.

TABLE 3
Primers for cDNA amplification.

| | |
|---|---|
| Primer 3 | /5Phos/AAATAATACGACTCACTATAGGTACACGAC |
| | GCTCTTCCGATCTG (SEQ ID 116) |
| | T7 Promoter; Alternative TSO sequence; |
| | Transcription start site |
| Primer 4a | GTCTCGTGGGCTCGGAGATGTGTATAAGAGACAG- |
| | nnnnnn-GCTGGACCACAGCCGCAGCGTCATGAG |
| | (SEQ ID 117) |
| | Reverse primer for whole alpha chain, |
| | I7 adapter |
| Primer 4b | GTCTCGTGGGCTCGGAGATGTGTATAAGAGACAG- |
| | nnnnnn-AATCCTTTCTCTTGACCATGGC |
| | (SEQ ID 118) |
| | Reverse primer for whole beta chain, |
| | I7 adapter |

All sequences shown 5′ to 3′. The sequences -nnnnnn- (SEQ ID NO: 104), represent a reverse complement of a Barcoded Index (BCI). A six nucleotide sequence is shown, which may have a sequence of any one of SEQ ID Nos. 1-96 of Table 1. However, any suitable barcode sequence may be used which may have a different number of nucleotides than 6, as described herein.

Using Primers 3 and 4a: a fragment (A2) is expected to result, having a sequence:

(SEQ ID NOS. 119 and 136)
/5Phos/AAATAATACGACTCACTATAGGTACACGACGCTCTTCC
GATCTG --- TCR alpha---nnnnnn-
CTGTCTCTTATACACATCTCCGAGCCCACGAGAC.

Using Primers 3 and 4b: a fragment (B2) is expected to result, having a sequence:

(SEQ ID NOS. 120 and 136)
/5Phos/AAATAATACGACTCACTATAGGTACACGACGCTCTTCC
GATCTG --- TCR beta---nnnnnn-
CTGTCTCTTATACACATCTCCGAGCCCACGAGAC.

The desired PCR amplicons 1020 can now be pooled (as they are tagged with their respective BCIs (nnnnnn (SEQ ID NO: 104))). Cleanup is performed using commercial spin column purification (e.g. Qiagen PCR clean up kit) or gel purification using methods common to the art.

In Vitro Transcription.

The amplicons 1020 from the PCR amplification step are used as a template to make single stranded RNA 1030 with the sequences respective to the above PCR fragments. In vitro transcription methods are common to those familiar with the art, and commercial kits such as Ampliscribe T7 Flash (Lucigen, Catalog No. ASF3257) is used according to manufacturer's directions, with a final template concentration of 4 ng/ul of transcription reaction, for 2.5 hr. at 37° C., followed by DNaseI digestion to remove the DNA template. Alternatively, non-commercial mixtures of transcription reaction reagents may be devised and used, as may be envisioned by one of skill. RNA from these reactions are cleaned up using commercial spin column purification, denaturing gel purification or bead purification, using methods common to the art (e.g., Agencourt AMPure beads (Beckman A63881) or -Agencourt AMPure beads (RNA quality) (Beckman A63987)).

Using fragment A2 for a template provides RNA having a sequence:

(SEQ ID NOS. 121 and 137)
GGUACACGACGCUCUUCCGAUCUG ---r(TCR beta V(D)J)---
GUGUUCCCACCCGAGGUCGC-rNrNrNrNrNrN-
CUGUCUCUUAUACACAUCUCCGAGCCCACGAGAC,

where the BCI is also transcribed to rNrNrNrNrNrN.

Using fragment B2 for a template provides RNA having a sequence:

(SEQ ID NOS. 122 and 138)
GGUACACGACGCUCUUCCGAUCUG ---r(TCR beta)---
rNrNrNrNrNrN- CUGUCUCUUAUACACAUCUCCGAGCCCACGAGAC.

Chemical Fragmentation of RNA:

The RNA 1030 transcribed above is randomly fragmented using a buffer (e.g. 200 mM Tris-Acetate, pH 8.1, 500 mM KOAc, 150 mM MgOAc). There are a number of different buffer conditions that can induce RNA fragmentation that include some/all of: pH 8-8.5, high temperature, high Mg concentrations or other commercially available products. The conditions may be empirically determined and will vary depending on the reagents used for fragmentation. The resulting RNA fragments 1040 have a length between 300-500 bp (if only sequencing the V(D)J region) or 300-1000 for full length TCR. Fragmentation can be stopped at a selected time point using a neutralization buffer (e.g. 0.05M EDTA, Tris-acetate pH 8.0). The desired fragments 1040 are cleaned up using gel/bead purification (e.g. Agencourt AMPure beads (Beckman A63881) or -Agencourt AMPure beads (RNA quality) (Beckman A63987)), using methods known to those in the art. In some experiments, some unfragmented RNA is saved for later.

Reverse Transcription of Fragmented and Full Length RNAs of TCRs.

Fragmented RNA 1040 is used as a template for a reverse transcription (template switching method) reaction analogous to the RT reaction used to generate the genomic pool of cDNA from total mRNA. For fragmented RNAs, use Primers 5 (1012) and 6 (1014) (sequences shown in Table 4) as shown in reaction complex 1040, to provide barcoded and fragmented DNA 1050. In a separate reaction, using the unfragmented TCR RNAs 1030 as a template, a standard reverse transcription reaction is performed with Primer 6 only.

TABLE 4
Primers for reverse transcription.

| | |
|---|---|
| Primer 5 | /5Biosg/ AGCAGCCGTCGCAGTCTACACATATTCTCTGTCr |
| | GrGrG (SEQ ID NO. 123) |
| | Template switching oligo that adds I5 |
| | adapter back to 5′ fragmented end of RNA |
| Primer 6 | GTCTCGTGGGCTCGGAGATGTGTATAAGAGACAG |
| | (SEQ ID NO. 124) |
| | Reverse primer that binds to all RNAs and |
| | amplifies from the 17 adapter |

After reverse transcription, the first strands (both reactions) are amplified (only the strands that contain the BCI) using Primers 6 and 7 (Table 5), as shown in complex 1055.

TABLE 5
Primers for amplification.

| | |
|---|---|
| Primer 7 | TCGTCGGCAGCGTCAGATGTGTATAAGAGACAG |
| | (SEQ ID NO. 125) |
| | Amplifies the first strand from the reverse |
| | transcription reactions (17 adapter) |

Mix ˜3% of the full-length amplified RT reaction from above with the product of the fragmented amplified RT reaction to ensure the 5′ end of the TCR is sufficiently captured in the sequencing step. Alternatively, or additionally, Primer sequences 4a/b could be modified to amplify only the V(D)J region of the TCRs, in vitro transcribed, fragmented and reverse transcribed (as above) to generate a more even distribution of reads across the TCR, particularly the V(D)J region. However, sufficient coverage of the whole TCR is attainable provided the RNA is not over fragmented and each sample has sufficient sequencing depth. The pool of barcoded TCR fragments 1050 are now indexed using PCR and Illumina Nextera based indexed adapter primers, as shown in reaction complex 1055, to provide indexed and barcoded amplified DNA fragment library 1060. (With nearly ˜400 unique Nextera indexes (1016) (Illumina 96 Index Set A (FC-131-2001), B (FC-131-2002), C (FC-131-2003) and D (FC-131-2004))), this allows one to pool multiple TCR libraries where: Maximum individual TCRs sequenced=number of unique Illumina indexes X number of unique BCIs.

Sequencing.

This method may be used for 150×150 paired end reads on an Illumina Miseq (MiSeq Reagent Kit v2 (300 cycle) (Illumina MS-102-2002)). Data analysis first requires demultiplexing the Illumina indexes (if more than one is used), followed by sorting each BCI that identifies each sample, per index. After adapter trimming, etc. the reads may be assembled into the full length TCR (or TCR V(D)J region).

### Example 3. Single End Random Fragmentation Sequencing Using 3′ Anchored Sequencing of Variable 5′ Regions. SERFc-Seq: Single End Random Fragmentation by Enzymatic Fragmentation of Amplified DNA and Circularization

A schematic representation of this experiment is shown in FIGS. 11A-C.

Human primary T cells were used as in Example 2, and a TCR cDNA library 1110 was produced as described above, and containing reverse primer sequence 1102 and TSO sequence 1104.

Creating Bottom Strand ssDNA TCR with Adapters and 5′ Linker.

Whole (full length) TCR sequences are amplified in reaction complex 1115 using primers 8 (1106) and 9a/b (9a is for the alpha chain, 9b is for the beta chain (1108)). Primers 9a/b are used with the same BCI for each sample. Individual TCRs are now barcoded (1120) and can be pooled at approx. equal molar ratios. Add linker to the bottom strand using PCR with Primer 8 (1106) and Primer 10 (1112) in complex 1125 to obtain amplicons 1130. Clean up the PCR reaction using methods common to the art. Digest the top (phosphorylated) strand using commercial lambda exonuclease for 90 min. at 37° C., followed by a clean up method, such as bead or column purification common to the art. (e.g., Agencourt AMPure beads (Beckman A63881) or -Agencourt AMPure beads (RNA quality) (Beckman A63987)) to obtain linker adapted 1135 containing TCR full sequences.

TABLE 6
Primers for amplification.

| | |
|---|---|
| Primer | /5Phos/TCGTCGGCAGCGTCAGATGTGTATAAGAGACAG- |
| 8 | TACACGACGCTCTTCCGATCTG (SEQ ID NO. 126) |
| | Alternative TSO sequence; I5 adapter |
| Primer | GTCTCGTGGGCTCGGAGATGTGTATAAGAGACAG-nnnnnn- |
| 9a | GCTGGACCACAGCCGCAGCGTCATGAG (SEQ ID NO. 127) |
| | Reverse primer for whole alpha chain, I7 |
| | adapter |
| Primer | GTCTCGTGGGCTCGGAGATGTGTATAAGAGACAG-nnnnnn- |
| 9b | GAAATCCTTTCTCTTGACCATGGC (SEQ ID NO. 128) |
| | Reverse primer for whole beta chain, I7 |
| | adapter |
| Primer | CTGTCTCTTATACACATCTGACGCTGCCGACGA/iSp18/CA |
| 10 | CTC A/iSp18/-GTCTCGTGGGCTCGGAGATGTGTATAAGA |
| | GACAG (SEQ ID NOS. 129 and 124) |
| | Linker = /iSp18/CA CTC A/iSp18/ I5 adapter |
| | I7 adapter |

All sequences 5′ to 3′

The sequence nnnnnn (SEQ ID NO: 104) (ICB) represents the reverse complement of BCI Barcoded index. The barcode shown here has six nucleotides but a barcode may have any suitable number of nucleotides as described herein. The index used in the examples below is AAAACT (SEQ ID NO: 1). The linker is /iSp18/CA CTC A/iSp18/

The full TCR is amplified with I5 and I7 adapters from cDNA, to produce:

(SEQ ID NOS. 130 and 139)
5′-pTCGTCGGCAGCGTCAGATGTGTATAAGAGACA [symbol] [symbol] [symbol]
-TCR-AAAACT-

[symbol]

[symbol]

[symbol]

[symbol]

(SEQ ID NOS. 131 and 135)
3′- [symbol] [symbol] [symbol] [symbol]
ATGTGCTGCGAGAAGGCT AGAC -TCR-TTTTGA-

[symbol]

[symbol]

[symbol]

[symbol]

Dilute and use PCR to add the linker to the bottom strand:

(SEQ ID NOS. 132 and 139)
5′-pTCGTCGGCAGCGTCAGATGTGTATAAGAGACAG- [symbol] [symbol] [symbol]
TCR-AAAACT- [symbol] [symbol] [symbol] [symbol]
-3′
(SEQ ID NOS. 133, 135 and 129)
3′- [symbol] [symbol] [symbol] [symbol] -
ATGTGCTGCGAGAAGGCTAGAC -TCR-TTTTGA- [symbol] [symbol] [symbol] [symbol]
-linker-
AGCAGCCGTCGCAGTCTACACATATTCTCTGTC-5′

Use lambda exonuclease to digest the top strand, and leave behind only:

(SEQ ID NOS. 134, 135 and 129)
3′- [symbol] [symbol] [symbol] [symbol] -
ATGTGCTGCGAGAAGGCTAGAC -TCR-TTTTGA- [symbol] [symbol] [symbol] [symbol]
-linker-
AGCAGCCGTCGCAGTCTACACATATTCTCTGTC-5′

Enzymatic Fragmentation of DNA and Circularization.

This step takes the ssDNA bottom strand 1135 and fragments it randomly with DNaseI (New England Bioloabs, M0303S), or an alternative endonuclease that targets ssDNA. Save a portion of the undigested ssDNA for spiking the circularization reaction. The conditions of this fragmentation may be empirically determined and will vary depending on the conditions used for fragmentation. The fragments 1140 are sized to be ˜300-1000 bp in length. Assuming random endonuclease activity and a single cut per strand, there is about a 95% probability that the endonuclease will cut upstream of the BCI under conditions when the endonuclease is at a much lower molar ratio than the DNA fragment. After fragmentation, gel purify the desired fragment size range using methods known to the art, in order to prepare for circularization with Circligase™ ssDNA ligase (Epicenter CL4111K). Mix ˜3% (by conc.) of the unfragmented ssDNA 1135 from above with the gel purified fragments 1140. Circularize the ssDNA strands using circligase using manufacturer protocols to obtain circularized fragments 1150. The portions of the fragmented ssDNA 1152 that circularize, but do not contain the i5 and i7 adapters, will not index and/or amplify in the indexing PCR.

ssDNA fragments (only the fragment that contains the whole region downstream of the BCI is shown for clarity):

(SEQ ID NOS. 135 and 129)
3′-Fragment of TCR-TTTTGA- [symbol] [symbol] [symbol] [symbol]
-linker-
AGCAGCCGTCGCAGTCTACACATATTCTCTGTC-5′

Circligase will circularize the ssDNA fragments as shown in FIG. 11C.

Indexing and Amplification of the Circularized TCR Library.

The pool of circularized TCR fragments 1150 is indexed (1114) and amplified using PCR and Illumina Nextera based indexed adapter primers. (With nearly ˜400 unique Nextera indexes ((Illumina 96 Index Set A (FC-131-2001), B (FC-131-2002), C (FC-131-2003) and D (FC-131-2004))), this allows one to pool multiple TCR libraries where: Max. individual TCRs sequenced=number of Illumina indexes X number of BCIs.

Indexing and amplifying library off the circular template. The first few cycles of PCR are linear amplification to obtain linear product, then both forward and reverse amplification leads to a double stranded amplified library 1160 as shown in FIG. 11C.

Sequencing.

The library may be sequenced via 150×150 paired end reads on an Illumina Miseq (MiSeq Reagent Kit v2 (300 cycle) (Illumina MS-102-2002)). Data analysis proceeds via demultiplexing the Illumina indexes (if more than one are used), followed by sorting each BCI that identifies each sample, per index. After adapter trimming, the reads are assembled the into the full length TCR (or TCR V(D)J region).

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner. Furthermore, where reference is made herein to a list of elements (e.g., elements a, b, c), such reference is intended to include any one of the listed elements by itself, any combination of less than all of the listed elements, and/or a combination of all of the listed elements. Also, as used herein, the terms a, an, and one may each be interchangeable with the terms at least one and one or more. It should also be noted, that while the term step is used herein, that term may be used to simply draw attention to different portions of the described methods and is not meant to delineate a starting point or a stopping point for any portion of the methods, or to be limiting in any other way.

### List of Some Embodiments

1. A method of preparing a nucleic acid library for sequencing, comprising; obtaining nucleic acid comprising RNA from a biological cell; synthesizing nucleic acid (e.g., complementary nucleic acid) from one or more of the RNA nucleic acids; amplifying the synthesized (e.g., complementary) nucleic acids; fragmenting or tagmenting the amplified nucleic acids, thereby providing a plurality of differentially truncated nucleic acids; amplifying and adding adapters to the plurality of differentially truncated nucleic acids, thereby providing a library of DNA for 5′ or 3′ anchored sequencing, wherein the DNA library comprises a plurality of differentially truncated DNA sample sequences.

2. The method of embodiment 1, wherein the DNA library comprises a plurality of DNA sequences comprising differentially 5′ truncated sequences, each having the same 3′ sequence as the other differentially 5′ truncated DNA sample sequences of the plurality.

3. The method of embodiment 1, wherein the DNA library comprises a plurality of DNA sequences comprising differentially 3′ truncated sequences, each having the same 5′ sequence as the other differentially 3′ truncated DNA sample sequences of the plurality.

4. A method of sequencing a nucleic acid library, comprising: sequencing the DNA library of any one of embodiments 1-3; tiling read sequences corresponding to at least one RNA nucleic acid; and reconstructing a full-length sequence of the at least one RNA nucleic acid.

5. A method of preparing a nucleic acid library for sequencing, comprising: obtaining nucleic acid comprising mRNA molecules from a biological cell; synthesizing cDNA from one or more of the mRNA molecules; amplifying the cDNA, thereby providing amplified DNA molecules, wherein each of the amplified DNA molecules comprises a first portion having a 5′ terminus and a first priming sequence proximal to the 5′ terminus, a third portion comprising the 3′ terminus and a second priming sequence proximal to the 3′ terminus, and a second portion comprising a sequence of interest corresponding to a cDNA sequence, wherein the second portion is disposed between the 3′ end of the first portion and the 5′ end of the third portion, wherein the second portion comprises a 5′ region having an unknown nucleic acid sequence and a 3′ region having a known nucleic acid sequence; and tagmenting the amplified DNA molecules, thereby providing a plurality of 5′ truncated DNA molecules, each truncated DNA molecule of the plurality comprising a 5′ portion comprising a third priming sequence, the third portion of a corresponding amplified DNA molecule, and a second portion consisting of a truncated sequence of interest; wherein the plurality of 5′ truncated DNA molecules comprises the nucleic acid library.

6. The method of embodiment 5, wherein each of the 5′ truncated DNA molecules further comprises a first barcode sequence.

7. The method of embodiment 6, wherein the first barcode sequence is located between the 3′ end of the second portion of the 5′ truncated DNA molecules and the 5′ end of the third portion of the 5′ truncated DNA molecules.

8. The method of embodiment 6 or 7, wherein the first barcode sequence is unique for mRNA isolated from the biological cell.

9. The method of any one of embodiments 5 to 8, wherein synthesizing the cDNA is performed with a nested Template Switching Oligonucleotide (TSO).

10. The method of any one of embodiments 5 to 9, wherein tagmenting further comprises inserting an adapter, thereby providing the 5′ third priming sequence.

11. The method of embodiment 10, wherein tagmenting further comprises inserting a second barcode, wherein the second barcode is disposed 3′ to the third priming sequence and 5′ to the truncated sequence of interest.

12. The method of any one of embodiments 5 to 11, further comprising amplifying the 5′ truncated DNA molecules.

13. The method of embodiment 12, wherein amplification of the 5′ truncated DNA molecules is performed with a gene specific 3′ primer.

14. The method of embodiment 13, wherein the gene specific 3′ primer primes the 5′ truncated DNA molecules at a location within the second portion, at a known gene specific sequence, thus providing a 3′ anchoring point for amplification.

15. The method of embodiment 13, wherein the 3′ anchoring point for amplification may be at a location other than a 3′ terminus of the known nucleic acid sequence of the cDNA sequence.

16. The method of any one of embodiments 12 to 15, wherein the amplification of the 5′ truncated DNA molecules adds a fourth priming sequence to the third portion, and wherein the third and the fourth priming sequences comprise adapter sequences configured for parallel sequencing.

16A. The method of any one of embodiments 12-15, wherein the amplification of the 5′ truncated DNA molecules may replace the third portion with a third portion comprising a fourth priming sequence, and the third and the fourth priming sequences may include adapter sequences configured for parallel sequencing.

17. The method of any one of embodiments 5 to 16 or 16A, wherein the second portions of the 5′ truncated DNA molecules range in length randomly less than a full-length of the 5′ region having the unknown nucleic acid sequence.

18. The method of any one of embodiments 5 to 17, wherein the nucleic acid library comprises a gene specific library.

19. The method of any one of embodiments 5 to 18, wherein the nucleic acid library comprises a library encoding a TCR or BCR sequence.

20. The method of any one of embodiments 5 to 19, wherein the TCR or BCR library comprises both heavy and light chain sequences.

21. The method of any one of embodiments 5 to 20, wherein obtaining the mRNA molecules comprises capturing mRNA with a capture oligonucleotide having a 3′ terminal dTVI oligonucleotide sequence.

22. The method of any one of embodiments 5 to 21, wherein obtaining the mRNA molecules comprises capturing the mRNA molecules to a capture object.

23. The method of embodiment 22, wherein capturing the mRNA molecules to the capture object is performed at a location disposed within a microfluidic device.

24. The method of embodiment 23, wherein the location at which the mRNA molecules are captured to the capture object comprises an isolation region of a sequestration pen.

25. A method of sequencing a nucleic acid library, comprising: sequencing the DNA library of any one of embodiments 5 to 24; tiling read sequences corresponding to at least one mRNA molecule; and reconstituting a full-length sequence of the at least one RNA molecule.

26. The method of embodiment 25, wherein the full-length mRNA molecule comprises a TCR or BCR oligonucleotide sequence.

27. The method of embodiment 26, wherein the TCR or BCR oligonucleotide sequence is a heavy chain or a light chain oligonucleotide sequence.

28. The method of any one of embodiments 25 to 27, wherein the read sequences are about 75 bp in length.

29. A method of preparing a nucleic acid library for sequencing, comprising: obtaining nucleic acid comprising mRNA molecules from a biological cell; synthesizing cDNA from one or more of the mRNA molecules; amplifying the cDNA to produce amplified DNA molecules, wherein each of the amplified DNA molecules comprises a first portion having a 5′ terminus and a RNA polymerase promoter sequence proximal to the 5′ terminus, a third portion comprising a 3′ terminus and a priming sequence proximal to the 3′ terminus, and a second portion corresponding to a cDNA sequence, wherein the second portion is disposed between the 3′ end of the first portion and the 5′ end of the third portion, and wherein the cDNA sequence of the second portion comprises a 5′ region having an unknown nucleic acid sequence and a 3′ region having a known nucleic acid sequence; transcribing the amplified DNA molecules to provide transcribed RNA molecules, each transcribed RNA molecule comprising a sequence of interest consisting of a copy of the second portion of a corresponding amplified DNA molecule, and a sequence consisting of a copy of the third portion of the corresponding amplified DNA molecule; fragmenting a portion of the transcribed RNA molecules, thereby providing a plurality of 5′ truncated RNA molecules, each truncated RNA molecule of the plurality comprising a 5′ portion consisting of a truncated sequence of interest and a 3′ portion comprising the 3′ priming sequence; and reverse transcribing the plurality of 5′ truncated RNA molecules, thereby providing a plurality of library DNA molecules, each library DNA molecule comprising a 5′ terminus that includes a second priming sequence, a 3′ terminus that includes the 3′ priming sequence, and a sequence disposed between the 5′ terminus and the 3′ terminus corresponding to a truncated sequence of interest.

30. The method of embodiment 29, wherein the 5′ portion of each of the plurality of 5′ truncated RNA molecules comprises a 5′ region having an unknown nucleic acid sequence and a 3′ region having at least a portion of a known nucleic acid sequence.

31. The method of embodiment 30, wherein the 5′ region of each 5′ truncated RNA molecule is truncated at the 5′ end of the unknown sequence (i.e., of the second portion of a corresponding amplified DNA molecule).

32. The method of any one of embodiments 29 to 31, wherein each of the amplified DNA molecules further comprises a barcode sequence.

33. The method of embodiment 32, wherein the barcode sequence is located between the 3′ end of the second portion and the 5′ end of the third portion of each amplified DNA molecule.

34. The method of embodiment 32 or 33, wherein the barcode is unique for mRNA isolated from the biological cell.

35. The method of any one of embodiments 29 to 34, wherein the 3′ region of the second portion of the amplified DNA molecules is shorter than a complete known DNA sequence for a gene specific DNA product of the mRNA.

36. The method of any one of embodiments 29 to 35, wherein each library DNA molecule of the plurality comprises the same portion of the known 3′ region of the cDNA.

37. The method of any one of embodiments 29 to 36, wherein synthesizing the cDNA comprises reverse transcribing the mRNA molecules.

38. The method of any one of embodiments 29 to 37, wherein synthesizing the cDNA comprises using a nested Template Switching Oligonucleotide.

39. The method of any one of embodiments 29 to 38, wherein amplifying the cDNA comprises amplifying with a gene specific 3′ primer.

40. The method of embodiment 39, wherein the gene specific primer primes the cDNA at a location corresponding to a known gene specific sequence, thus providing a 3′ anchoring point for amplification.

41. The method of any one of embodiments 29 to 40, wherein transcribing the amplified DNA is performed using a RNA polymerase.

42. The method of any one of embodiments 29 to 41, wherein reverse transcribing the plurality of 5′ truncated RNA molecules further comprises inserting an adaptor and thereby providing the second priming sequence.

43. The method of any one of embodiments 29 to 42, wherein the priming sequence and the second priming sequence comprise adapter sequences configured for parallel sequencing.

44. The method of embodiment 42, wherein inserting the adaptor comprises performing PCR subsequent to reverse transcribing the plurality of 5′ truncated RNA molecules

45. The method of embodiment 44, wherein performing PCR subsequent to reverse transcribing the plurality of 5′ truncated RNA molecules further comprises adding sequencing indices to the 5′ and the 3′ termini of the amplified molecules.

46. The method of any one of embodiments 29 to 45, wherein reverse transcribing the plurality of 5′ truncated RNA molecules further comprises reverse transcribing a second portion of the transcribed RNA molecules, wherein the second portion of the transcribed RNA molecules has not been fragmented.

47. The method of any one of embodiments 29 to 46, wherein fragmenting the transcribed RNA molecules comprises chemically fragmenting.

48. The method of any one of embodiments 29 to 47, wherein each library DNA molecule of the plurality comprises a 5′ truncated region of unknown sequence, wherein the 5′ truncated region ranges in length (e.g., randomly less than a full length of the 5′ region of unknown nucleic acid sequence from the corresponding cDNA).

49. The method of any one of embodiments 29 to 48, wherein the plurality of library DNA molecules comprises a gene specific library of DNA molecules.

50. The method of any one of embodiments 29 to 49, wherein the plurality of library DNA molecules comprises a library of DNA molecules encoding a TCR or BCR sequence.

51. The method of embodiment 50, wherein the TCR or BCR DNA library comprises both heavy and light chain sequences.

52. The method of any one of embodiments 29 to 51, wherein obtaining the mRNA molecules comprises capturing an mRNA molecule with a capture oligonucleotide having a 3′ terminal dTVI oligonucleotide sequence.

53. The method of any one of embodiments 29 to 52, wherein obtaining the mRNA molecules further comprises capturing the mRNA molecules to a capture object.

54. The method of embodiment 53, wherein capturing the mRNA molecules to the capture object is performed at a location disposed within a microfluidic device.

55. The method of embodiment 54, wherein the location at which the mRNA molecules are captured to the capture object comprises an isolation region of a sequestration pen.

56. A method of sequencing a nucleic acid library, comprising: sequencing the DNA library DNA of any one of claims 26-50; tiling read sequences corresponding to at least one mRNA molecule; and reconstructing a full length sequence of the at least one mRNA molecule.

57. The method of embodiment 56, wherein the full length sequence of the at least one mRNA molecule comprises a TCR or BCR oligonucleotide sequence.

58. The method of embodiment 57, wherein the TCR or BCR oligonucleotide sequence is a heavy chain or a light chain oligonucleotide sequence.

59. The method of any one of embodiments 56 to 58, wherein the read sequences are about 75 bp in length.

60. The method of preparing a nucleic acid library for sequencing of any one of embodiments 1 to 3; 5 to 24; 29 to 58; or 73 to 91, wherein the barcode has a sequence of any one SEQ ID NOS. 1-96.

61. The method of sequencing a nucleic acid library of any one of embodiments 4; 25 to 28; 56 to 59; or 92 to 95, wherein the barcode has a sequence of any one of SEQ ID NOS. 1-96.

62. A kit for preparing a nucleic acid library, comprising: a RNA capture oligonucleotide; a gene specific primer; and a fragmenting reagent.

63. The kit of embodiment 62, wherein the RNA capture oligonucleotide has a dTVI sequence at a 3′ terminus.

64. The kit of embodiment 62 or 63, wherein the RNA capture oligonucleotide comprises a priming sequence at or proximal to a 5′ terminus.

65. The kit of any one of embodiments 62 to 64, wherein the gene specific primer is specific for a TCR or a BCR sequence.

66. The kit of embodiment 65, wherein the TCR or BCR gene specific primer primes both heavy and light chain sequences of the TCR or BCR gene.

67. The kit of any one of embodiments 62 to 66, wherein the fragmenting reagent is a chemical fragmentation reagent or an enzymatic fragmentation reagent.

68. The kit of embodiment 67, wherein the chemical fragmentation reagent is a divalent cation.

69. The kit of embodiment 68, wherein the divalent cation comprises magnesium or zinc.

70. The kit of embodiment 67, wherein the enzymatic fragmentation reagent comprises a non-specific nuclease, a restriction endonuclease, or a tagmentation reagent comprising a transposase.

71. The kit of embodiment 70, wherein the non-specific nuclease is DNase 1.

72. The kit of any one of embodiments 62 to 71, further comprising reverse transcriptase.

73. A method of preparing a nucleic acid library for sequencing, comprising: obtaining nucleic acid comprising mRNA molecules from a biological cell; synthesizing cDNA from one or more of the mRNA molecules; amplifying the cDNA to produce amplified DNA molecules, wherein each of the amplified DNA molecules comprises a first portion having a 5′ terminus and a first priming sequence proximal to the 5′ terminus, a third portion comprising a 3′ terminus and a second priming sequence proximal to the 3′ terminus, and a second portion corresponding to a cDNA sequence, wherein the second portion is disposed between the 3′ end of the first portion and the 5′ end of the third portion, and wherein the cDNA sequence of the second portion comprises a 5′ region having an unknown nucleic acid sequence and a 3′ region having a known nucleic acid sequence; further amplifying the amplified DNA molecules to insert a specialized priming sequence having a third priming sequence linked via a linker containing at least one non-nucleotide moiety to a fourth priming sequence; and digesting a top strand of the further amplified DNA molecules, thereby producing a bottom strand linker-modified amplified DNA molecule, wherein the bottom strand linker-modified amplified DNA comprises a first portion having a 5′ terminus wherein the third priming sequence is at the 5′ terminus and is linked via a linker containing at least one non-nucleotide moiety to the fourth priming sequence, a third portion having a 3′ terminus and comprising a complement to the first priming sequence; and a second portion comprising a sequence of interest corresponding to a cDNA sequence, wherein the second portion is disposed between the 3′ end of the first portion and the 5′ end of the third portion, and wherein the second portion comprises a complement to the 5′ region having an unknown nucleic acid sequence and a complement to the 3′ region having a known nucleic acid sequence; fragmenting at least a first portion of the bottom strand linker-modified amplified DNA molecules, thereby providing a plurality of fragments truncated within the complement of the 5′ region of the cDNA corresponding to the RNA molecule, and the third portion of a corresponding amplified DNA molecule; circularizing each of the plurality of truncated bottom strand DNA molecules, to provide a plurality of circularized DNA molecules, each comprising a fragment corresponding to a truncated sequence of interest and the specialized priming sequence, wherein the third priming sequence remains linked via the linker containing at least one non-nucleotide moiety to the fourth priming sequence; amplifying the plurality of circularized DNA molecules, wherein the fourth priming sequence comprises a binding site for a reverse primer sequence and the third priming sequence comprises a a forward primer sequence, thereby providing a plurality of 5′ truncated DNA library molecules, each 5′ truncated DNA library molecule comprising a first portion comprising the third priming sequence, wherein the third priming sequence is proximal to a 5′ terminus, a third portion comprising the fourth priming sequence, wherein the fourth priming sequence is proximal to a 3′ terminus, and a second portion comprising a 5′ truncated sequence of interest.

74. The method of embodiment 73, wherein each of the amplified DNA molecules further comprises a barcode sequence.

75. The method of embodiment 74, wherein the barcode sequence is located between the 3′ end of the second portion of the amplified DNA molecule and the 5′ end of the third portion of the amplified DNA molecule.

76. The method of embodiment 74 or 75, wherein the barcode is unique for mRNA isolated from the biological cell.

77. The method of any one of embodiments 73 to 76, wherein amplifying the cDNA to provide amplified DNA molecules is performed using a nested Template Switching Oligonucleotide (TSO).

78. The method of any one of embodiments 73 to 77, wherein amplifying the cDNA to provide amplified DNA molecules is performed with a gene specific 3′ primer.

79. The method of embodiment 78, wherein the gene specific primer primes the cDNA at a location within a known gene specific sequence, thus providing a 3′ anchoring point for amplification.

80. The method of embodiment 79, wherein the 3′ anchoring point for amplification is at a location other than a 3′ terminus of the known sequence of the cDNA.

81. The method of any one of embodiments 73 to 80, wherein the third and the fourth priming sequences comprise adapter sequences configured for parallel sequencing.

82. The method of any one of embodiments 73 to 81, wherein fragmenting comprises enzymatically fragmenting.

83. The method of any one of embodiments 73 to 82, wherein the 5′ truncated DNA molecules range in length, randomly less than a full length of the 5′ region having the unknown nucleic acid sequence.

84. The method of any one of embodiments 73 to 83, wherein each 5′ truncated DNA library molecule of the plurality comprises the same 3′ region having the known nucleic acid sequence.

85. The method of any one of embodiments 73 to 84, wherein the plurality of 5′ truncated DNA library molecules comprises a gene specific 5′ truncated DNA library.

86. The method of any one of embodiments 73 to 85, wherein the plurality of 5′ truncated DNA library molecules comprise a 5′ truncated DNA library encoding a TCR or BCR sequence.

87. The method of embodiment 86, wherein the TCR or BCR 5′ truncated DNA library comprises both heavy and light chain sequences.

88. The method of any one of embodiments 73 to 87, wherein obtaining the mRNA molecules comprises capturing mRNA molecules with a capture oligonucleotide having a 3′ terminal TₙVI oligonucleotide sequence.

89. The method of any one of embodiments 73 to 88, wherein obtaining the mRNA molecules comprises capturing the mRNA molecules to a capture object.

90. The method of embodiment 89, wherein capturing the mRNA molecules to the capture object is performed at a location disposed within a microfluidic device.

91. The method of embodiment 90, wherein the location at which the mRNA molecules are captured to the capture object comprises an isolation region of a sequestration pen.

92. A method of sequencing a nucleic acid library, comprising: sequencing the plurality of 5′ truncated DNA molecules of any one of embodiments 73 to 91; tiling read sequences corresponding to at least one mRNA molecule; and reconstructing a full length sequence of the at least one mRNA molecule.

93. The method of embodiment 92, wherein the at least one mRNA molecule comprises a TCR or BCR oligonucleotide sequence.

94. The method of embodiment 93, wherein the TCR or BCR oligonucleotide sequence is a heavy chain or a light chain oligonucleotide sequence.

95. The method of any one of embodiments 92 to 94, wherein the read sequences are about 150 bp in length.

1. A method of preparing a nucleic acid library for sequencing, comprising;
obtaining nucleic acid comprising RNA from a biological cell;
synthesizing a sequence of nucleic acids from one or more of the RNA nucleic acids;
amplifying the synthesized sequence of nucleic acids;
fragmenting or tagmenting the amplified nucleic acids, thereby providing a plurality of differentially truncated nucleic acids;
amplifying and adding adapters to the plurality of differentially truncated nucleic acids, thereby providing a library of DNA for 5′ or 3′ anchored sequencing, wherein the DNA library comprises a plurality of differentially truncated DNA sample sequences.

2. The method of claim 1, wherein the DNA library comprises a plurality of differentially 5′ truncated DNA sample sequences, each having the same 3′ sequence as the other differentially 5′ truncated DNA sample sequences of the plurality.

3. The method of claim 1, wherein the DNA library comprises a plurality of DNA sequences comprising differentially 3′ truncated DNA sample sequences, each having the same 5′ sequence as the other differentially 3′ truncated DNA sample sequences of the plurality.

4. (canceled)

5. A method of preparing a nucleic acid library for sequencing, comprising:
obtaining nucleic acid comprising mRNA molecules from a biological cell;
synthesizing cDNA from one or more of the mRNA molecules;
amplifying the cDNA, thereby providing amplified DNA molecules, wherein each of the amplified DNA molecules comprises a first portion having a 5′ terminus and a first priming sequence proximal to the 5′ terminus, a third portion comprising the 3′ terminus and a second priming sequence proximal to the 3′ terminus, and a second portion comprising a sequence of interest corresponding to a cDNA sequence, wherein the second portion is disposed between the 3′ end of the first portion and the 5′ end of the third portion, wherein the second portion comprises a 5′ region having an unknown nucleic acid sequence and a 3′ region having a known nucleic acid sequence; and
tagmenting the amplified DNA molecules, thereby providing a plurality of 5′ truncated DNA molecules, each 5′ truncated DNA molecule of the plurality comprising a 5′ portion comprising a third priming sequence, the third portion of a corresponding amplified DNA molecule, and a second portion consisting of a truncated sequence of interest;
wherein the plurality of 5′ truncated DNA molecules comprises the nucleic acid library.

6. The method of claim 5, wherein each of the 5′ truncated DNA molecules further comprises a first barcode sequence.

7. The method of claim 6, wherein the first barcode sequence is located between the 3′ end of the second portion of the 5′ truncated DNA molecules and the 5′ end of the third portion of the 5′ truncated DNA molecules.

8. The method of claim 6, wherein the first barcode sequence is unique for mRNA isolated from the biological cell.

9. The method of claim 5, wherein synthesizing the cDNA is performed with a nested Template Switching Oligonucleotide (TSO).

10. The method of claim 5, wherein tagmenting further comprises inserting an adapter, thereby providing the 5′ third priming sequence.

11. The method of claim 10, wherein tagmenting further comprises inserting a second barcode, wherein the second barcode is disposed 3′ to the third priming sequence and 5′ to the truncated sequence of interest.

12. The method of claim 5, further comprising amplifying the 5′ truncated DNA molecules.

13. The method of claim 12, wherein amplification of the 5′ truncated DNA molecules is performed with a gene specific 3′ primer.

14. The method of claim 13, wherein the gene specific 3′ primer primes the 5′ truncated DNA molecules at a location within the second portion, at a known gene specific sequence, thus providing a 3′ anchoring point for amplification.

15. The method of claim 12, wherein the amplification of the 5′ truncated DNA molecules adds a fourth priming sequence to the third portion, and wherein the third and the fourth priming sequences comprise adapter sequences configured for parallel sequencing.

16. The method of claim 5, wherein the second portions of the 5′ truncated DNA molecules range in length randomly less than a full-length of the 5′ region having the unknown nucleic acid sequence.

17. The method of claim 5, wherein the nucleic acid library comprises a gene specific library.

18. The method of claim 5, wherein the nucleic acid library comprises a library encoding a TCR or BCR sequence.

19. The method of claim 5, wherein the TCR or BCR library comprises both heavy and light chain sequences.

20. The method of claim 5, wherein obtaining the mRNA molecules comprises capturing mRNA with a capture oligonucleotide having a 3′ terminal dTVI oligonucleotide sequence.

21. The method of claim 5, wherein obtaining the mRNA molecules comprises capturing the mRNA molecules to a capture object.

22.-56. (canceled)

57. A kit for preparing a nucleic acid library, comprising:
a RNA capture oligonucleotide;
a gene specific primer; and
a fragmenting reagent.

58. The kit of claim 57, wherein the RNA capture oligonucleotide has a dTVI sequence at a 3′ terminus.

59. The kit of claim 57, wherein the RNA capture oligonucleotide comprises a priming sequence at or proximal to a 5′ terminus.

60. The kit of claim 57, wherein the gene specific primer is specific for a TCR or a BCR sequence.

61.-67. (canceled)
